(19) United States
(12) Patent Application Publication
(10) Pub. No.: US 2025/0144156 A1
(43) Pub. Date: May 8, 2025

(54) MICROBIAL CONSORTIA AND USES THEREOF

(71) Applicant: The University of Chicago, Chicago, IL (US)

(72) Inventors: Arjun S. Raman, Western Springs, IL (US); Ana Rita Almeida de Oliveira, Chicago, IL (US); Seppe Kuehn, Chicago, IL (US)

(21) Appl. No.: 18/915,109

(22) Filed: Oct. 14, 2024

Related U.S. Application Data
(60) Provisional application No. 63/543,908, filed on Oct. 12, 2023.

(51) Int. Cl. A61K 35/74; A61K 35/00; A61K 35/742; A61K 35/745

(52) U.S. Cl. CPC A61K 35/74; A61K 35/742; A61K 35/745; A61K 2035/11

(57) ABSTRACT

The present document relates to microbial consortia and uses thereof.

### CROSS-REFERENCE TO RELATED APPLICATIONS

This application claims priority to U.S. Provisional Application Ser. No. 63/543,908, filed Oct. 12, 2023, which is incorporated by reference herein in its entirety.

### SEQUENCE LISTING

The instant application contains a Sequence Listing which has been submitted in ST26 format and is hereby incorporated by reference in its entirety. Said ST26 copy, created on 8 Oct. 2024, is named “ARCD.P0842US_sequence_listing.xml” and is 15,106 bytes in size.

### TECHNICAL FIELD AND BACKGROUND

### I. Technical Field

The present document relates to microbial consortia and uses thereof.

### II. Background

Engineering communities of microbes for desired functions (“synthetic ecology”) is of fundamental importance and holds great practical promise for addressing many problems facing humanity^{5,8,9}. So called “top-down” approaches—reducing an already functional, whole microbiome to key microbes—and “bottom-up” approaches—designing communities one bacterium at a time—have found success in creating functional communities¹⁰⁻¹³. However, the ability to create new communities that predictably execute a desired function according to principles of design, i.e. deriving “generative” models of microbiome engineering, remains immensely challenging. In large part, this is due to the daunting complexity of ecosystems: they are comprised of many parts that interact with each other and the environment in dynamic and unintuitive manners to give rise to emergent, collective function^{6,7-14-17}.

### SUMMARY

The present disclosure relates to microbial consortia and uses thereof. Certain aspects of the present disclosure relate to populations of bacteria that are identified using methods provided herein, including methods that utilize machine learning techniques.

Also disclosed herein are compositions comprising a bacterial population comprising at least one bacterial strain. Also disclosed herein are bacterial compositions, isolated bacterial compositions, pharmaceutical compositions, non-natural compositions, therapeutic bacterial compositions, synthetic formulation compositions, live biotherapeutic product compositions, fecal transplant compositions, and/or probiotic product compositions, where the composition comprises at least one bacterial strain. In certain aspects, the composition (which may be any composition disclosed herein) is for treatment, or prevention of bacterial infections, or for improving the general health and well-being of a subject in need thereof. Also provided herein are methods of using these compositions for the treatment or prevention of infections, or for general health, or well-being of a subject in need thereof.

In some aspects, the composition comprises one or more bacterial strains. In some aspects, the composition comprises a bacterial population comprising one or more bacterial strains. The bacterial strains can include any bacterial strain disclosed herein. In certain aspects, the bacterial strain(s) is/are a Clostridium innocuum strain, a Clostridium symbiosum strain, a Collinsella aerofaciens strain, an Escherichia coli strain, a Bacteroides xylanisolvens strain, a Lacrimispora celerecrescens strain (or Clostridium celerecrescens strain), a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, a Blautia producta strain, or any combination thereof. In certain aspects, the bacterial strain includes any of the bacterial strains disclosed herein.

In certain aspects, the Clostridium innocuum strain comprises Clostridium innocuum strain MSK.7.7. In certain aspects, the Clostridium symbiosum strain comprises Clostridium symbiosum strain MSK.7.21. In certain aspects, the Collinsella aerofaciens strain comprises Collinsella aerofaciens strain MSK.1.10. In certain aspects, the Escherichia coli strain comprises Escherichia coli strain MSK.19.28. In certain aspects, the Bacteroides xylanisolvens strain comprises Bacteroides xylanisolvens strain MSK.20.34. In certain aspects, the Lacrimispora celerecrescens strain comprises Lacrimispora celerecrescens strain MSK.15.50. In certain aspects, the Bacteroides caccae strain comprises Bacteroides caccae strain MSK.18.53. In certain aspects, the Blautia faecis strain comprises Blautia faecis strain MSK.17.74. In certain aspects, the Blautia obeum strain comprises Blautia obeum strain MSK.13.37. In certain aspects, the Clostridium scindens strain comprises Clostridium scindens strain MSK.5.24. In certain aspects, the Bifidobacterium strain comprises Bifidobacterium sp. MSK.13.1. In certain aspects, the Megasphaera massiliensis strain comprises Megasphaera massiliensis strain MSK.20.21. In certain aspects, the Coprococcus comes strain comprises Coprococcus comes strain MSK.17.59. In certain aspects, the Mitsuokella jalaludinii strain comprises Mitsuokella jalaludinii strain MSK.21.47. In certain aspects, the Blautia producta strain comprises Blautia producta strain MSK.4.17.

In certain aspects, the Clostridium innocuum strain is Clostridium innocuum strain MSK.7.7. In certain aspects, the Clostridium symbiosum strain is Clostridium symbiosum strain MSK.7.21. In certain aspects, the Collinsella aerofaciens strain is Collinsella aerofaciens strain MSK.1.10. In certain aspects, the Escherichia coli strain is Escherichia coli strain MSK.19.28. In certain aspects, the Bacteroides xylanisolvens strain is Bacteroides xylanisolvens strain MSK.20.34. In certain aspects, the Lacrimispora celerecrescens strain is Lacrimispora celerecrescens strain MSK.15.50. In certain aspects, the Bacteroides caccae strain is Bacteroides caccae strain MSK.18.53. In certain aspects, the Blautia faecis strain is Blautia faecis strain MSK.17.74. In certain aspects, the Blautia obeum strain is Blautia obeum strain MSK.13.37. In certain aspects, the Clostridium scindens strain is Clostridium scindens strain MSK.5.24. In certain aspects, the Bifidobacterium strain is Bifidobacterium sp. MSK.13.1. In certain aspects, the Megasphaera massiliensis strain is Megasphaera massiliensis strain MSK.20.21. In certain aspects, the Coprococcus comes strain is Coprococcus comes strain MSK.17.59. In certain aspects, the Mitsuokella jalaludinii strain is Mitsuokella jalaludinii strain MSK.21.47. In certain aspects, the Blautia producta strain is Blautia producta strain MSK.4.17.

In some aspects, the bacterial population is an isolated bacterial population. In some aspects, the bacterial population comprises, consists of, or consists essentially of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or more or any range derivable therein, different bacterial strains. In some aspects, the bacterial population comprises, consists of, or consists essentially of the bacterial strains of Block 1. In some aspects, the bacterial population comprises, consists of, or consists essentially of the bacterial strains of Block 2. In some aspects, the bacterial population comprises, consists of, or consists essentially of the bacterial strains of Block 3. In some aspects, the bacterial population comprises, consists of, or consists essentially of the bacterial strains of Block 4. In some aspects, the bacterial population comprises, consists of, or consists essentially of the bacterial strains of Block 5. In some aspects, the bacterial population comprises, consists of, or consists essentially of the bacterial strains of Block 1 and Block 5. In some aspects, the bacterial population comprises, consists of, or consists essentially of one or more bacterial strains of Block 1, Block 2, Block 3, Block 4, Block 5, or any combination thereof.

Also disclosed are compositions comprising the bacterial strains of Block 1. Also disclosed are compositions comprising the bacterial strains of Block 2. Also disclosed are compositions comprising the bacterial strains of Block 3. Also disclosed are compositions comprising the bacterial strains of Block 4. Also disclosed are compositions comprising the bacterial strains of Block 5. Also disclosed are compositions comprising the bacterial strains of Block 1 and Block 5. Also disclosed are compositions comprising the bacterial strains of Block 1, Block 2, Block 3, Block 4, Block 5, or any combination thereof.

Any of the compositions disclosed herein can also comprise a pharmaceutically acceptable excipient. Any of the compositions disclosed herein can be formatted for oral administration, rectal administration, or as a fecal transplant.

Disclosed herein are methods for treating, methods for prophylactically treating, methods for treating an infection, methods for prophylactically treating an infection, methods for treating pneumonia, methods of prophylactically treating pneumonia, modulating one or more short chain fatty acids and/or one or more amino acids in a patient, methods of altering a microbiome in a patient, methods of killing pathogenic bacteria, and methods of introducing commensal bacteria to a patient. The method can comprises one or more steps including any of the following: administering a therapeutically effective amount of a composition (including any composition described herein) to a patient, administering a therapeutically effective amount of commensal bacteria to a patient, detecting bacteria in a biological sample from a patient, detecting one or more microbiome bacterial strains in a patient, detecting an infection in a patient, diagnosing a patient with an infection, administering a second therapeutic composition (such as an antibiotic) to a patient, and monitoring an infection in a patient; wherein the patient is a patient described herein. In some aspects, the administration treats or prophylactically treats the patient.

In some aspects, the patient has symptoms of or has been diagnosed with pneumonia. In some aspects, the pneumonia is community-acquired pneumonia. In some aspects, the pneumonia is caused by an Enterobacteriaceae sp. In some aspects, the pneumonia is caused by a drug resistant Enterobacteriaceae sp. or a multi-drug resistant Enterobacteriaceae sp. In some aspects, the patient has symptoms of or has been diagnosed with an infection. In certain aspects, the infection is caused by an Enterobacteriaceae sp. In some aspects, the infection is caused by a drug resistant Enterobacteriaceae sp. or a multi-drug resistant Enterobacteriaceae sp.

In certain aspects, the patient is administered a composition comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, or more or any range derivable therein, different bacterial strains. In certain aspects, the patient is administered a composition comprising the bacterial strains of Block 1. In certain aspects, the patient is administered a composition comprising the bacterial strains of Block 2. In certain aspects, the patient is administered a composition comprising the bacterial strains of Block 3. In certain aspects, the patient is administered a composition comprising the bacterial strains of Block 4. In certain aspects, the patient is administered a composition comprising the bacterial strains of Block 5. In certain aspects, the patient is administered a composition comprising the bacterial strains of Block 1 and Block 5. In certain aspects, the patient is administered a composition comprising one or more bacterial strains of Block 1, Block 2, Block 3, Block 4, Block 5, or any combination thereof.

Also disclosed are methods for designing therapeutic bacterial populations, machine learning methods, methods for identifying commensal bacteria, methods for designing pharmaceutical compositions, methods for designing a bacterial population for treating an infection, methods for identifying a bacterial population capable of inhibiting or killing a pathogenic bacteria. In some aspects, the method comprises one or more steps including any of the following: building a set of random synthetic bacterial communities from a bank of bacterial strains, testing each random synthetic bacterial community from the set of random synthetic bacterial communities for an ability to clear an undesired bacterial strain, determining, based on said testing, one or more designated microbial communities (DMCs) predicted to clear the undesired bacterial strain, and/or any other step disclosed herein.

In certain aspects, the bank of bacterial strains is designed by a method comprising one or more of the following steps: identifying each bacterial strain in a microbiome from a sample, determining a genomic similarity between each bacterial strain, and including bacterial strains that maximize genomic diversity into the bank of bacterial strains. In certain aspects, the sample comprises a biological sample from a healthy subject. In some aspects, the sample comprises a fecal sample. In some aspects, one or more of the random synthetic bacterial community comprises between 5 to 50 bacterial strains. In some aspects, one or more of the random synthetic bacterial community comprises between 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein) bacterial strains.

In certain aspects, the testing comprises in vitro testing. For example, the testing may comprise inoculating the random synthetic bacterial community with the undesired bacteria on a bacterial culture medium and determining the growth (or lack thereof) of the undesired bacteria. In certain aspects, the testing comprises in vivo testing. For example, the testing may comprise inoculating an animal with the random synthetic bacterial community and the undesired bacteria and determining the effect on the animal. In some aspects, the testing comprises one or more assays that test the ability of the random synthetic bacterial community's ability to suppress growth of the undesired bacterial strain, including any assay described herein. In some aspects, the testing comprises inoculating each random synthetic bacterial community individually with the undesired bacterial strain. In some aspects, the testing comprises applying, into a trained machine learning model, the bacterial composition each random synthetic bacterial community. For example, a machine learning model can be generated to predict the ability of a bacterial community to inhibit or kill an undesired bacteria using any of the machine learning algorithms disclosed herein. The bacterial makeup of the random synthetic bacterial community can be applied to the machine learning model to predict a likelihood of the random synthetic bacterial community to inhibit or kill the undesired bacteria.

In some aspects, the DMC comprise between 5 to 25 bacterial strains. In some aspects, the DMC comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 (or any range derivable therein) bacterial strains. In some aspects, the DMC comprises a co-variance lower than 0.5, 0.4, 0.3, 0.2, or 0.1%.

In some aspects, the undesired bacterial strain comprises a drug resistant, multi-drug resistant, or pathogenic bacterial strain. In some aspects, the undesired bacterial strain comprises an Enterobacteriaceae sp. In some aspects, the undesired bacterial strain comprises a Klebsiella sp., Klebsiella pneumoniae, or hypervirulent Klebsiella pneumoniae. In some aspects, the undesired bacterial strain comprises a carbapenem-resistant Enterobacteriaceae sp. or a carbapenem-resistant Klebsiella pneumoniae. In some aspects, the K. pneumoniae comprises K. pneumoniae strain MH258. In some aspects, the undesired bacteria is a bacterial strain known to a cause an infection. In some aspects, the undesired bacteria is a bacterial strain causing an infection in a patient. In some aspects, the DMC predicted to clear or reduce the presence of the undesired bacterial strain below 10, 8, 6, 4, 2, or 1 Log₁₀ colony forming units (CFU)/mL, or any value therebetween.

### Definitions

The terms “a” and “an” are defined as one or more unless this disclosure explicitly requires otherwise. For example, the use of the word “a” or “an” when used in conjunction with the term “comprising” may mean “one,” but it is also consistent with the meaning of “one or more,” “at least one,” and “one or more than one.”

As used herein, “or” is inclusive and not exclusive, unless expressly indicated otherwise or indicated otherwise by context, and can have the same meaning as “and/or.” To illustrate, A, B, or C includes: A alone, B alone, C alone, a combination of A and B, a combination of A and C, a combination of B and C, or a combination of A, B, and C.

Throughout this application, the term “about” is used to indicate that a value includes the inherent variation of error for the measurement or quantitation method.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, a composition that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

As used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term).

Any configuration of any of compositions or methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

By “pharmaceutically acceptable excipient” is meant any ingredient other than a strain and having the properties of being non-toxic and non-inflammatory in a subject. Exemplary, non-limiting excipients include adjuvants, antiadherents, antioxidants, binders, carriers (e.g., cocoa butter, polyethylene glycol, or a suppository wax), coatings, compression aids, diluents, disintegrants, dispersing agents, dyes (colors), emollients, emulsifiers, fillers (diluents), film formers or coatings, flavors, fragrances, glidants (flow enhancers), isotonic carriers, lubricants, preservatives, printing inks, solvents, sorbents, stabilizers, suspensing or dispersing agents, surfactants, sweeteners, waters of hydration, waxes, or wetting agents. Any of the excipients can be selected from those approved, for example, by the United States Food and Drug Administration or other governmental agency as being acceptable for use in humans or domestic animals. Exemplary excipients include, but are not limited to alcohol, butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, glycerol, hydroxypropyl cellulose, hydroxypropyl methylcellulose, lactated Ringer's solution, lactose, magnesium stearate, maltitol, maltose, mannitol, methionine, methylcellulose, methyl paraben, microcrystalline cellulose, polyethylene glycol, polyol, polyvinyl pyrrolidone, povidone, pregelatinized starch, propyl paraben, retinyl palmitate, Ringer's solution, shellac, silicon dioxide, sodium carboxymethyl cellulose, sodium chloride injection, sodium citrate, sodium starch glycolate, sorbitol, starch (corn), stearic acid, stearic acid, sucrose, talc, titanium dioxide, vegetable oil, vitamin A, vitamin E, vitamin C, water, and xylitol.

As used herein, “administration,” “administering,” and variants thereof refers to introducing a composition into a subject and includes concurrent and sequential introduction of a composition. “Administration” can refer, e.g., to therapeutic, pharmacokinetic, diagnostic, research, placebo, and experimental methods. “Administration” also encompasses in vitro and ex vivo treatments. The introduction of a composition into a subject is by any suitable route, including orally, rectally, pulmonarily, intranasally, parenterally (intravenously, intramuscularly, intraperitoneally, or subcutaneously), intralymphatically, or topically. Administration includes self-administration and the administration by another. Administration can be carried out by any suitable route. A suitable route of administration allows the composition to perform its intended function.

As used herein, the terms “subject,” “individual,” and “patient” are used interchangeably herein and refer to any animal subject for whom diagnosis, treatment, or therapy is desired, particularly humans. In some aspects, the subject is a mammal (e.g., a human subject). In some aspects, the subject is a non-human mammal (e.g., mouse, rat, guinea pig, dog, cat, horse, cow, pig, rabbit, sheep, or non-human primate, such as a monkey, chimpanzee, or baboon).

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

As used herein, the terms “therapeutically effective amount” and “effective amount” are used interchangeably to refer to an amount of a composition that is sufficient to provide the intended benefit (e.g., prevention, prophylaxis, delay of onset of symptoms, or amelioration of symptoms of a disease). In prophylactic or preventative applications, an effective amount can be administered to a subject susceptible to, or otherwise at risk of developing a disease, disorder or condition to eliminate or reduce the risk, lessen the severity, or delay the onset of the disease, disorder or condition, including a biochemical, histologic and/or behavioral symptoms of the disease, disorder or condition, its complications, and intermediate pathological phenotypes.

As used herein, the terms “treat,” “treating,” and/or “treatment” include abrogating, substantially inhibiting, slowing or reversing the progression of a disorder, disease or condition, substantially ameliorating clinical symptoms of a disorder, disease or condition, or substantially preventing the appearance of clinical symptoms of a disorder, disease or condition, obtaining beneficial or desired clinical results. Treating further refers to accomplishing one or more of the following: (a) reducing the severity of the disorder, disease or condition); (b) limiting development of symptoms characteristic of the disorder, disease or condition(s) being treated; (c) limiting worsening of symptoms characteristic of the disorder, disease or condition(s) being treated; (d) limiting recurrence of the disorder, disease or condition(s) in subjects that have previously had the disorder, disease or condition(s); and (e) limiting recurrence of symptoms in subjects that were previously asymptomatic for the disorder, disease or condition(s). Beneficial or desired clinical results, such as pharmacologic and/or physiologic effects include, but are not limited to, preventing the disease, disorder or condition from occurring in a subject predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (e.g., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

By “isolated” is meant a material that is free to varying degrees from components which normally accompany it as found in its native state. “Isolate” denotes a degree of separation from original source or surroundings.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Ranges provided herein are understood to be shorthand for all of the values within the range. For example, a range of 1 to 50 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

Other features and advantages of the present document will be apparent from the following detailed description, the figures, and the claims.

### BRIEF DESCRIPTION OF THE DRAWINGS

The following drawings illustrate certain aspects of the features and advantages of this document. These aspects are not intended to limit the scope of the appended claims in any manner. Like reference symbols in the drawings indicate like elements.

FIG. 1 shows a non-limiting depiction of clustering strain-strain covariance scores that characterizes a relationship of the pair of bacterial strains in relation to suppressing drug-resistant Klebsiella pneumoniae bacteria, where the strain-strain covariance scores are represented as a matrix of numerical values.

FIG. 2 shows results of testing of Klebsiella pneumoniae (Kp) suppression in BHIS (brain heart infusion supplemented) media. The following designed microbial communities (DMCs) were tested for their ability to suppress Kp when co-cultured in BHIS media for 120 hours: DMC46 (including 46 strains), Block 1, Block 2, Block 3, Block 4, Block 5, and Block 1+Block 5 (also indicated as “synCom15”). Kp is denoted by “I,” DMC46 is denoted by “ii,” Block 1 is denoted by “iii,” and Block 1+Block 5 is denoted by “iv.”

FIG. 3A-3C show testing of Klebsiella pneumoniae (Kp) suppression in cecal extract media. FIG. 3A shows a non-limiting schematic showing processing of cecal media from (i) germ-free mice, or (ii) antibiotic-treated specific-pathogen free (SPF) mice. FIG. 3B shows results of testing of Kp suppression in cecal extract media made from germ-free mice. FIG. 3C shows results of testing of Kp suppression in cecal extract media made from mice treated with antibiotics. The following compositions were tested for their ability to suppress Kp when co-cultured in BHIS media for 120 hours: DMC46 (including 46 strains), Block 1, Block 2, Block 3, Block 4, Block 5, and Block 1+Block 5 (including 15 strains, also indicated as “synCom15”). Kp is denoted by “I,” DMC46 is denoted by “ii,” Block 1 is denoted by “iii,” and Block 1+Block 5 is denoted by “iv.”

FIG. 4 shows a schematic of a non-limiting experimental protocol for assessing Kp fecal abundance over a period of 21 days. In this protocol, specific pathogen free male mice (C5JBL6) can be singly housed, can be fed a normal mouse chow diet, and can be given broad spectrum antibiotics (metronidazole, neomycin, and vancomycin (MNV)) for three to five days, then infected with Kp, and then given three doses (gavage) by both nasogastric tube orally and fecal enema of either saline (PBS), a fecal microbial transplant (FMT), or one of two designed microbial communities (DMCs): e.g., DMC19 (including 19 strains) or synCom15 (having 15 strains from Block 1 and Block 5).

FIG. 5 shows results of testing of Kp fecal abundance after infection (at day 0). Mice were given three doses by both nasogastric tube orally and fecal enema of either saline (indicated as “Control (PBS)”), a fecal microbial transplant (indicated as “Control (FMT)”), or one of two designed microbial communities: DMC19 (including 19 strains) or synCom15 (having 15 strains from Block 1 and Block 5). Control (BPS) line is indicated by “i,” DMC15 line is indicated by “ii,” DMC19 line is indicated by “iii,” and control (FMT) like is indicated by “iv.”

FIG. 6A-6C shows presence of fatty acids and amino acids in cecal samples after treatment with Kp, top suppressive strains, bottom suppressive strains, and synCom15 (having 15 strains from Block 1 and Block 5). For each graph, the groups are represented by each bar from left to right: Kp, Top suppressive, Bottom suppressive, and Block 1+5.

FIGS. 7A-7F. A generative model for engineering communities that suppressed K. pneumoniae. FIG. 7A shows a workflow of a standard Design-Build-Test-Learn (DBTL) framework. Communities are designed (“D”) and built (“B”) from a strain bank, tested (“T”) for desired function, and a statistical model mapping community composition with function is learned (“L”). New communities are then designed based on the learned model and the process is iterated. FIG. 7B shows diversity of the full strain bank described at phylogenetic level of phylum. FIG. 7C shows a subset of strain bank used to make Designed Microbial Communities (DMCs) described at phylogenetic level of phylum. FIG. 7D shows a schematic for design of a five-member DMC. ‘Seed’ bacterium is a randomly chosen member of the strain bank. FIG. 7E shows engineered DMCs (rows) described by strain composition (columns). Dark colored squares denote that the strain is included in the DMC, whereas white squares denote that the strain is not included in the DMC. Each row is labeled by the number of strains within the DMC (‘Community complexity’) and the K. pneumoniae abundance after 120 hours of co-culture in BHIS media (‘K. pneumoniae abundance’). Rows are ordered by their ability to suppress K. pneumoniae after 120 hours of co-culture. “DMC1,” the last row, is K. pneumoniae in monoculture (‘Kp only’). FIG. 7F shows K. pneumoniae abundance predicted by a random forest (RF) model for 60 new DMCs not included in FIG. 7E (x-axis) versus K. pneumoniae abundance observed after 120 hours of co-culture with the 60 new DMCs (y-axis). The RF model was trained and validated to predict K. pneumoniae abundance after 120 hours of co-culture using only the designed strain presence-absence matrix in FIG. 7E as data.

FIGS. 8A-8E. Defining SynCom15 and evaluating its capacity to suppress K. pneumoniae across different environments. FIGS. 8A-8B show a histogram of predicted K. pneumoniae abundance for 100,000 in silico generated DMCs. Red arrow is a threshold of predicted K. pneumoniae suppression; DMCs to the left of the arrow were selected to create an alignment of 5,752 DMCs defined by their pattern of strain presence-absence as shown in FIG. 8B. Each in silico DMC was labeled by the predicted K. pneumoniae abundance after 120 hours of co-culture (green bar) as shown in FIG. 8B. FIG. 8C shows a graph depicting the contribution of each of the 5,752 DMCs onto the 461 principal component (PC46) of the matrix depicted in FIG. 8B (x-axis) versus predicted K. pneumoniae abundance associated with each DMC (y-axis). FIG. 8D shows the contribution of each strain onto PC46 (left panel). The right panel of FIG. 8D shows hierarchically clustered strain-strain matrix where each entry represents the similarity in contribution onto PC46 between two strains. Blocks 1 through 5 were defined according to the clustering pattern (colored dots in dendrogram). Bars in left panel were colored according to which Block each strain belonged. FIG. 8E shows a workflow for creating cecal extract media from germ-free (“GF”) and antibiotic treated specific pathogen free (“Ab-treated SPF”) mice (top panel). K. pneumoniae abundance (y-axis) is shown in the bottom panel for DMC46, all Blocks, and SynCom15 (darker shade) after 120 hours of co-culture in BHIS (left graph), GF cecal extract media (middle graph), and Ab-treated SPF cecal extract media (right graph). K. pneumoniae abundance after 120 hours of monoculture (‘Kp alone’) in each media is shown in black. Dashed line is detection limit of assay.

FIGS. 9A-9G. SynCom15 sustainably cleared K. pneumoniae in a pre-clinically relevant mouse model of infection. FIG. 9A shows a schematic of specific pathogen free (SPF) mice treated with metronidazole, neomycin, and vancomycin (MNV) (days 0-4), then infected with K. pneumoniae (“Kp gavage,” day 5), then given either a mouse fecal microbial transplant (FMT), saline (PBS), DMC46, Block 1, Block 2, or SynCom15 (days 7-9). Fecal samples were collected at select days delineated in the schematic as “Sampling” and mice were sacrificed after day 21. FIG. 9B shows median fecal abundance of K. pneumoniae (y-axis) versus time (x-axis). Vertical dashed lines on days 2, 3, and 4 reflect gavage of bacterial communities or controls (“Consortia or Controls”). Error bars indicate interquartile range. FIG. 9C shows engraftment statistics and relative presence of SynCom15 strains through the experiment. FIG. 9D shows Median Chao (top) and Shannon (bottom) diversity indices (y-axes) versus time (x-axes) for SPF mice treated with MNV, infected with K. pneumoniae (‘Kp’), and given PBS, FMT, or SynCom15. Error bars indicate interquartile range. Control (PBS) is indicated by “i,” control (FMT) is indicated by “ii,” DMC46 is indicated by “iii,” Block 1 is indicated by “iv,” Block 2 is indicated by “v,” and SynCom15 is indicated by “vi.” FIG. 9E shows a PCoA plot of fecal microbiota for SPF mice on day 0, 7, and 16 of experiment; colored shape is centroid for indicated cohort. FIG. 9F shows the distribution of average relative abundance for fecal microbiota through time (x-axis) for infected mice treated with FMT (left panel), PBS (middle panel), or SynCom15 (right panel). Distributions were defined spanning kingdom to genera-level descriptions. FIG. 9G shows the relative abundance of Bacteroides, Duncaniella, Paramuribaculum and Bifidobacterium genera that are differentially abundant amongst infected mice treated with FMT, saline, or SynCom15 prior to antibiotic treatment (day 0) and at day 16 after treatment (equivalent to 11 days after infection with K. pneumoniae). Statistical tests performed were two-way ANOVA; **p<0.01; ***p<0.001; ****p<0.0001.

FIGS. 10A-10C. Comparison of SynCom15 composition with composition of healthy human microbiomes. FIG. 10A shows a phylogenetic tree of genera present across fecal microbiomes of human donors. Highlighted genera are those found across SynCom15 strains (genera names are according to annotation by Metaphlan). FIG. 10B shows prevalence pattern for species of SynCom15 (rows) across donor fecal microbiomes (DFI is Duchossois Family Institute; columns). FIG. 10C shows a histogram of relative abundance for SynCom15 species (x-axis) across all fecal samples from population of healthy human donors.

FIGS. 11A-11E. Comparing metabolite-based and strain-based models of community design. FIG. 1A shows p-values for differential enrichment of metabolites between the 5 most suppressive DMCs and K. pneumoniae alone (x-axis), and p-values for differential enrichment of metabolites between the 5 least suppressive DMCs and K. pneumoniae alone (y-axis) (p-values computed by two-way ANOVA). Box indicates features that are significantly differentially enriched in the most suppressive DMCs but not in the least suppressive DMCs. Bar plots show distribution of normalized peak areas (y-axis) for each metabolite feature in the box (x-axes) for, from left to right for each graph, K. pneumoniae alone (left bar), the five most suppressive DMCs (second bar), the five least suppressive DMCs (third bar), and SynCom15 (right bar) at the 72, 96, and 120 hour culture timepoint. *p<0.05; **p<0.01; ****p<0.0001. FIG. 11B shows distributions of normalized peak areas (y-axes) of fatty acids and amino acids from fecal samples collected on Day 10 and Day 12 of mouse experiments shown in FIG. 9A for mice gavaged with saline (left dots) or SynCom15 (right dots). ***p<0.001. FIG. 11C shows a correlation between predicted K. pneumoniae from an RF model trained on metabolite profile of DMCs (x-axis) and observed K. pneumoniae abundance after 120 hours of co-culture with DMCs (y-axis). FIG. 11D shows a correlation between predicted K. pneumoniae abundance from RF model trained on metabolite profile of DMCs (x-axis) and RF model trained on pattern of strain presence-absence in DMCs (y-axis) (panel D). Dots shown are 60 “out of-sample” DMCs. FIG. 11E shows a structure of metabolite profiles for DMCs (PC1 vs. PC2) versus K. pneumoniae abundance after 120 hours of co-culture (z-axis) (left panel), and a structure of strain presence-absence for DMCs (PC1 vs PC2) versus K. pneumoniae abundance after 120 hours of co-culture (z-axis) (right panel). Each dot on the surfaces is a DMC; surfaces are interpolated.

FIG. 12 shows tree of the genera comprising all fecal microbiomes (annotations per Metaphlan). Fecal samples collected from 22 healthy human donors were subject to shotgun metagenomic sequencing (Example 3). Colored names represent the distribution of genera observed in the bank of 848 commensal strains.

FIG. 13 shows a matrix of strain by Prokka feature for all 848 gut commensals where entries are a “1” if the Prokka feature is present in the strain proteome or “0” if Prokka feature is absent in the strain proteome. Matrix was subjected to UMAP visualization; UMAP plot is shown in FIG. 7B, top panel.

FIGS. 14A-14B. FIG. 14A shows a workflow for an algorithm used to design DMCs. Communities with three bacterial strains shown as an example. Given three possible communities that could be created, the first step was to choose the mean dispersal of the lowest third of pairwise distances between strains for each community. In the example shown, the lowest third was equivalent to the minimum pairwise distance for each community due to the communities being comprised of only three strains (gray box). The second step was to the choose the community with the maximal dispersion per Step 1. In the case shown, “Community 1” would be chosen as a DMC for incorporation into a DBTL framework. FIG. 14B shows an average Shannon diversity (y-axis) versus number of species in DMCs (x-axis). The maximum possible Shannon diversity was set by the DMC containing all 46 strains used to engineer DMCs.

FIGS. 15A-15G. FIG. 15A shows a workflow for evaluating clearance capacity of DMCs for K. pneumoniae (“Kp”) in vitro (“BHIS” is Brain Heart Infused media supplemented with cysteine, “kan” is kanamycin). FIG. 15B shows a time course of K. pneumoniae abundance (y-axis) for all 96 communities shown in FIG. 7E. Solid line represents median, shaded area represents range. FIGS. 15C-15E show reproducibility of assay. FIG. 15C shows a correlation between the suppressive capacity of several different DMCs across two experimental replicates. FIG. 15D shows a time course of DMC containing all 46 bacterial strains (DMC46) across five experimental replicates. FIG. 15E shows the variation in three DMCs that were replicated within the 96 DMCs shown in FIG. 7E, and inset shows the variance in K. pneumoniae abundance for all three DMCs as a function of their respective community size. FIG. 15F shows K. pneumoniae abundance (y-axis) after co-culture with each DMC (x-axis) for 120 hours where the x-axis is ordered by most suppressive (left) to least suppressive (right) DMCs. FIG. 15G shows K. pneumoniae abundance (y-axis) after co-culture with each DMC (x-axis) for 120 hours where the x-axis is ordered by least complex (left) to most complex (right) DMCs. For plots from FIGS. 15F-15G, the black bar represents K. pneumoniae grown in monoculture in BHIS.

FIGS. 16A-16B. FIG. 16A shows feature importance scores for each strain of SynCom15 resulting from an RF model built on strain presence-absence. FIG. 16B shows feature importance scores (x-axis) for each strain (dots) versus the projection of each strain onto PC46 (y-axis) of matrix defined in FIG. 8B. Projection of strains onto PC46 are also shown in FIG. 8D, left panel.

FIG. 17 shows dynamics of SynCom15 strains (y-axes) through time (x-axes) after serial triple gavage of SynCom15 in SPF mice pre-treated with broad spectrum antibiotics (“MNV treatment”) and then infected with K. pneumoniae MH258 (“Kp”). Error bars represent ±1 standard deviation across cohorts of mice.

FIG. 18 shows hematoxylin and eosin stains of colon from infected mice given SynCom15.

FIGS. 19A-19D. FIG. 19A shows K. pneumoniae abundance (y-axis) when co-cultured with each of the SynCom15 strains (x-axis) for 120 hours in BHIS media. “K. pneumoniae alone” labels the K. pneumoniae abundance in monoculture for 120 hours in BHIS shown in FIG. 8E. “SynCom15” labels the K. pneumoniae abundance of K. pneumoniae in co-culture with SynCom15 for 120 hours in BHIS shown in FIG. 8E. “Detection limit” labels the lower limit of K. pneumoniae abundance for the assay. FIG. 19B shows a composition of (i) SynCom15; (ii) strains of SynCom15 that engrafted mice (“SynCom15_1”); (iii) strains of SynCom15 that engrafted mice without E. coli (“SynCom15_2”); (iv) strains of SynCom15 found in mice across all timepoints of the experiment shown in FIG. 9 (“SynCom15_3”); and (v) SynCom15 without E. coli (“SynCom15_4”). FIGS. 19C-19D shows all communities defined in FIG. 19B that were assayed for K. pneumoniae clearance in BHIS (FIG. 19C) and GF cecum content (FIG. 19D) media. Black bars indicate K. pneumoniae abundance in BHIS and GF cecum content when cultured alone.

FIG. 20 shows a workflow for detecting metabolite features distinguishing most and least suppressive DMCs when co-cultured with K. pneumoniae compared to K. pneumoniae in monoculture. The distribution of K. pneumoniae abundances after 120 hours of co-culture with DMCs was used to select the five “most suppressive” and “least suppressive” DMCs (boxes in barplot) for further metabolic analysis.

FIGS. 21A-21B. FIG. 21A shows the predictive capacity of an RF model trained on metabolite profiles of DMCs for the training set of data. FIG. 21B shows the predictive capacity of an RF model trained on metabolite profiles of DMCs for the validation set of data.

FIG. 22 shows metabolite profiles after 120 hours of co-culture with K. pneumoniae and K. pneumoniae (“Kp”) abundance for communities delineated in inset. Metabolite features were chosen as those that most contributed to variance along the main principal component (PC1) of metabolite variation across all 81 DMCs used to train the RF model. Surface shown represents the same surface shown in FIG. 11E.

FIGS. 23A-23B. FIG. 23A shows a formula for computing “Effective compression.” FIG. 23B shows compression from a starting point (bank of 848 gut commensal strains) to SynCom15 across two steps performed serially. Step 1 was using the genomes of the strain bank to reduce complexity from 848 to 46 strains. Step 2 was performing DBTL+ and statistical inference to engineer SynCom15. For the “I” value in Step 1, the total information used were the full genomes of all 848 strains. As a conservative measure of compressive power, each base pair for all genomes was considered as a unique piece of information. The total number of basepairs for the commensal strain bank was 8.65×10¹¹. For the “I” value in Step 2, 96 DMCs were tested for their capacity to suppress K. pneumoniae; 12 DMCs were tested to validate the reproducibility of the assay for evaluating the suppressive capacity of a DMC; 60 DMCs were tested as “out-of-sample” communities to test the predictive capacity of the RF model built on DMC presence-absence; 21 experiments were performed to evaluate the capacity of all Blocks, DMC46, and SynCom15 to suppress K. pneumoniae in BHIS, germ-free cecal extract, and antibiotic-treated specific-pathogen free (Ab-SPF) cecal extract media; 39 experiments were performed to evaluate the capacity of saline, an FMT, Block 1, Block 2, and SynCom15. In total, this yielded 228 experiments performed to compress the space of 2⁴⁶/2 to SynCom15.

### DETAILED DESCRIPTION

The present document relates to microbial consortia and uses thereof. In some aspects, a microbial consortium can be designed to provide a therapeutic benefit (e.g., for treatment of a disease, such as pneumonia, infection, or any others described herein). In turn, the microbial consortium can be provided as a composition including a plurality of identified strains.

In some aspects, the composition includes one or more strains (e.g., 2, 3, 4, 5, 6, 7, 8, 9, 10, 11, 12, 13, 14, or 15 strains) of the following: a Clostridium innocuum strain, a Clostridium symbiosum strain, a Collinsella aerofaciens strain, an Escherichia coli strain, a Bacteroides xylanisolvens strain, a Lacrimispora celerecrescens strain (or Clostridium celerecrescens strain), a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, and a Blautia producta strain.

In some aspects, the composition includes one or more strains (e.g., 2, 3, 4, 5, 6, 7, 8, 9, 10, 11, 12, 13, 14, or 15 strains) of the following: Clostridium innocuum strain MSK.7.7, Clostridium symbiosum strain MSK.7.21, Collinsella aerofaciens strain MSK.1.10, Escherichia coli strain MSK.19.28, Bacteroides xylanisolvens strain MSK.20.34, Lacrimispora celerecrescens strain MSK.15.50, Bacteroides caccae strain MSK.18.53, Blautia faecis strain MSK.17.74, Blautia obeum strain MSK.13.37, Clostridium scindens strain MSK.5.24, Bifidobacterium sp. MSK.13.1, Megasphaera massiliensis strain MSK.20.21, Coprococcus comes strain MSK.17.59, Mitsuokella jalaludinii strain MSK.21.47, and Blautia producta strain MSK.4.17.

In some aspects, the composition includes one or more strains (e.g., 2, 3, 4, 5, 6, 7, 8, 9, 10, 11, 12, 13, 14, or 15 strains), wherein at least one strain comprises a sequence having at least 90% (e.g., 91%, 92%, 93%, 94%, 95%, 96%, 97%, 98%, 99%, 99.5%, or 99.9%) sequence identity to any of the sequences identified in Table 1 (e.g., by GenBank Accession Number).

In some aspects, the composition includes one or more strains (e.g., 2, 3, 4, 5, 6, 7, 8, 9, 10, 11, 12, 13, 14, or 15 strains), wherein each strain comprises a sequence having at least 90% (e.g., 91%, 92%, 93%, 94%, 95%, 96%, 97%, 98%, 99%, 99.5%, or 99.9%) sequence identity to any of the sequences identified in Table 1 (e.g., by GenBank Accession Number). For example and without limitation, if the composition includes five strains, then each of the five strains has a sequence having at least 90% (e.g., 91%, 92%, 93%, 94%, 95%, 96%, 97%, 98%, 99%, 99.5%, or 99.9%) sequence identity to five of the sequences identified in Table 1 (e.g., by GenBank Accession Number).

In some aspects, the composition comprises, consists of, or consists essentially of one or more strains (e.g., 2, 3, 4, 5, 6, 7, 8, 9, 10, 11, 12, 13, 14, or 15 strains) from Supplemental Table 2. In some aspects, the composition comprises, consists of, or consists essentially of Collinsella aerofaciens, Bifidobacterium sp., Blautia obeum, Lacrimispora celerecrescens, Coprococcus comes, Blautia faecis, Bacteroides caccae, Escherichia coli, Megasphaera massiliensis, Bacteroides xylanisolvens, Mitsuokella jalaludinii, Blautia producta, Clostridium scindens, Clostridium symbosium, Clostridium innocuum, Odoribacter splanchnicus, Parabacteroides merdae, Sellimonas intestinalis, Clostridium cochlearium, Bacteroides eggerthii, Ruminococcus lactaris, Acidaminococcus intestini, Bacteroides cellulosilyticus, Enterocloster clostridioformis, Phocaeicola massiliensis, Bacteroides uniformis, Prevotella stercorea, Megasphaera elsdenii, Fusicatenibacter saccharivorans, Bifidobacterium breve, Dorea formicigenerans, Veillonella ratti, Ruminococcus faecis, Blautia luti, Bifidobacterium pseudocatenulatum, Lachnospiraceae sp., Blautia hansenii, Erysipelatoclostridium ramosum, Blautia wexlerae, Lactococcus lactis, Phocaeicola dorei, Bacteroides ovatus, Bacteroides thetaiotaomicron, Megamonas funiformis, Bifidobacterium adolescentis, Parabacteroides sp., or a combination thereof.

In some aspects, the composition includes at least 15 strains (i.e., a Clostridium innocuum strain, a Clostridium symbiosum strain, a Collinsella aerofaciens strain, an Escherichia coli strain, a Bacteroides xylanisolvens strain, a Lacrimispora celerecrescens strain (or Clostridium celerecrescens strain), a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, and a Blautia producta strain).

In some aspects, the composition includes at least 15 strains: Clostridium innocuum strain MSK.7.7, Clostridium symbiosum strain MSK.7.21, Collinsella aerofaciens strain MSK.1.10, Escherichia coli strain MSK.19.28, Bacteroides xylanisolvens strain MSK.20.34, Lacrimispora celerecrescens strain MSK.15.50, Bacteroides caccae strain MSK.18.53, Blautia faecis strain MSK.17.74, Blautia obeum strain MSK.13.37, Clostridium scindens strain MSK.5.24, Bifidobacterium sp. MSK.13.1, Megasphaera massiliensis strain MSK.20.21, Coprococcus comes strain MSK.17.59, Mitsuokella jalaludinii strain MSK.21.47, and Blautia producta strain MSK.4.17.

In some aspects, the composition includes at least 5 strains: a Clostridium innocuum strain, a Clostridium symbiosum strain, a Collinsella aerofaciens strain, an Escherichia coli strain, and a Bacteroides xylanisolvens strain.

In some aspects, the composition includes at least 5 strains: Clostridium innocuum strain MSK.7.7, Clostridium symbiosum strain MSK.7.21, Collinsella aerofaciens strain MSK.1.10, Escherichia coli strain MSK.19.28, and Bacteroides xylanisolvens strain MSK.20.34.

In some aspects, the composition includes at least 10 strains: a Lacrimispora celerecrescens strain (or Clostridium celerecrescens strain), a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, and a Blautia producta strain.

In some aspects, the composition includes at least 10 strains: Lacrimispora celerecrescens strain MSK.15.50, Bacteroides caccae strain MSK.18.53, Blautia faecis strain MSK.17.74, Blautia obeum strain MSK.13.37, Clostridium scindens strain MSK.5.24, Bifidobacterium sp. MSK.13.1, Megasphaera massiliensis strain MSK.20.21, Coprococcus comes strain MSK.17.59, Mitsuokella jalaludinii strain MSK.21.47, and Blautia producta strain MSK.4.17.

Certain aspects relate to compositions comprising a bacteria. The bacteria may include bacterial strains from one or more of Block 1, Block 2, Block 3, Block 4, Block 5, or subsets of bacteria selected thereof. In some aspects, the therapeutic composition comprises, consists of, or consists essentially of a bacteria, including bacteria from Block 1, Block 2, Block 3, Block 4, Block 5, or subsets of bacteria selected thereof (or any other bacteria disclosed herein), in a unit dosage.

The unit dosage may be any dosage sufficient for the desired effect of the therapeutic composition. The unit dosage may be the bacteria combined in an amount that is not found in nature. In some aspects, the bacterial populations, including one or more of Block 1, Block 2, Block 3, Block 4, Block 5, or subsets of bacteria selected thereof (or any other bacteria disclosed herein), are enriched, isolated, or purified to an amount and/or concentration that is not found in nature. In some aspects the unit dosage comprises between 1×10³ to 9×10¹⁶ colony forming units (CFU) of the bacteria. In some aspects the unit dosage comprises at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria. In another aspect, the disclosure relates to compositions comprising an isolated or purified population of one or more of Block 1, Block 2, Block 3, Block 4, Block 5, or subsets of bacteria selected thereof (or any other bacteria disclosed herein). Therapeutic compositions and methods of administering one or more of Block 1, Block 2, Block 3, Block 4, Block 5, or subsets of bacteria selected thereof (or any other bacteria disclosed herein) may involve such unit dosages. Moreover, a unit dosage may be given multiple times over a time period as discussed below.

In some aspects, the composition comprises a bacteria, including any bacteria disclosed herein such as one or more of Block 1, Block 2, Block 3, Block 4, Block 5, or subsets of bacteria selected thereof (or any other bacteria disclosed herein), and a second composition, which can include a metabolite (including any metabolite described herein), antibiotic, and/or immunosuppressant.

The therapeutic compositions can be formulated for administration, including as pharmaceutical formulations, e.g., formulated for oral administration; suppository administration; or injection such as via the intravenous, intramuscular, subcutaneous, or intraperitoneal routes. Such compositions can be prepared as either liquid solutions or suspensions; solid forms suitable for use to prepare solutions or suspensions upon the addition of a liquid prior to injection can also be prepared; and, the preparations can also be emulsified.

In certain aspects, the therapeutic composition, which may include a bacteria, including any bacteria disclosed herein, and a saponin, including any saponin disclosed herein, is formulated for oral administration. The formulation for oral administration may comprise a pill, capsule, suspension, drink, or the like. In some aspects, the saponin is administered through food.

In some aspects, the therapeutic composition comprises fecal transplant. The fecal transplant may comprise one or more of Block 1, Block 2, Block 3, Block 4, Block 5, or subsets of bacteria selected thereof (or any other bacteria disclosed herein). In some aspects, the fecal matter is administered in a dose of 50 g. In some aspects, the fecal matter is administered in a dose of at least, at most, or exactly 10, 15, 20, 25, 30, 35, 40, 45, 50, 55, 60, 65, 70, 75. 80, 85, 90, 95, 100, 110, 120, 130, 140, 150, 160, 170, 180, 190, 200, 210, 220, 230, 240, 250, 260, 270, 280, 290, 300, 325, 350, 375, or 400 g (or any derivable range therein). In certain aspects, the fecal transplant comprises fecal matter collected from a patient that has not received an antibiotic in 1, 2, 3, 4, 5, 6, 7, 8, 9, 10 days, weeks, months, and/or years (and any range derivable therein) prior to collecting the fecal matter. In certain aspects, the fecal transplant comprises fecal matter that comprises a measurable amount of one or more of Block 1, Block 2, Block 3, Block 4, Block 5, or subsets of bacteria selected thereof (or any other bacteria disclosed herein). In certain aspects, the fecal transplant comprises fecal matter that comprises a therapeutically effective amount of one or more of Block 1, Block 2, Block 3, Block 4, Block 5, or subsets of bacteria selected thereof (or any other bacteria disclosed herein).

The pharmaceutical formulations suitable for injectable use include sterile aqueous solutions or dispersions; formulations including, for example, aqueous propylene glycol; and sterile powders for the extemporaneous preparation of sterile injectable solutions or dispersions. In certain aspects, the formulation is stable under the conditions of manufacture and storage and preserved against the contaminating action of non-therapeutic microorganisms.

A pharmaceutical composition or formulation can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of unintended microorganisms can be brought about by various anti-bacterial and anti-fungal agents, for example, parabens, chlorobutanol, phenol, sorbic acid, thimerosal, and the like. In certain aspects, the formulation includes isotonic agents, for example, sugars or sodium chloride. Prolonged absorption of the injectable compositions can be brought about by the use in the compositions of agents delaying absorption, for example, aluminum monostearate and gelatin.

Injectable solutions may be prepared by incorporating the active compounds in the required amount in the appropriate solvent with various other ingredients enumerated above, as required. In certain aspects encompassing powders for the preparation of injectable solutions, the therapeutic composition(s) are vacuum-dried and/or freeze-dried, which yield a powder of the active ingredient, plus any additional desired ingredient.

The present disclosure also provides a pharmaceutical composition comprising one or more microbial cultures as described above. The bacterial species therefore are present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure are preferably formulated for oral administration, other routes of administration can be employed, however, including, but not limited to, intracolonic, subcutaneous, intramuscular, intradermal, transdermal, intraocular, intraperitoneal, mucosal, vaginal, rectal, and intravenous.

In another aspect, the disclosed composition may be prepared as a suppository. The suppository may include but is not limited to the bacteria and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the composition may be prepared as a tablet. The tablet may include the bacteria and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the composition may be formed as food or drink or, alternatively, as an additive to food or drink, wherein an appropriate quantity of bacteria is added to the food or drink to render the food or drink the carrier.

The compositions of the present disclosure may further comprise one or more prebiotics known in the art, such as lactitol, inulin, or a combination thereof.

In some aspects, the composition may further comprise a food or a nutritional supplement effective to stimulate the growth of one or more bacteria, including any bacteria disclosed herein, present in the gastrointestinal tract of the subject. In some aspects, the nutritional supplement is produced by a bacterium associated with a healthy human gut microbiome.

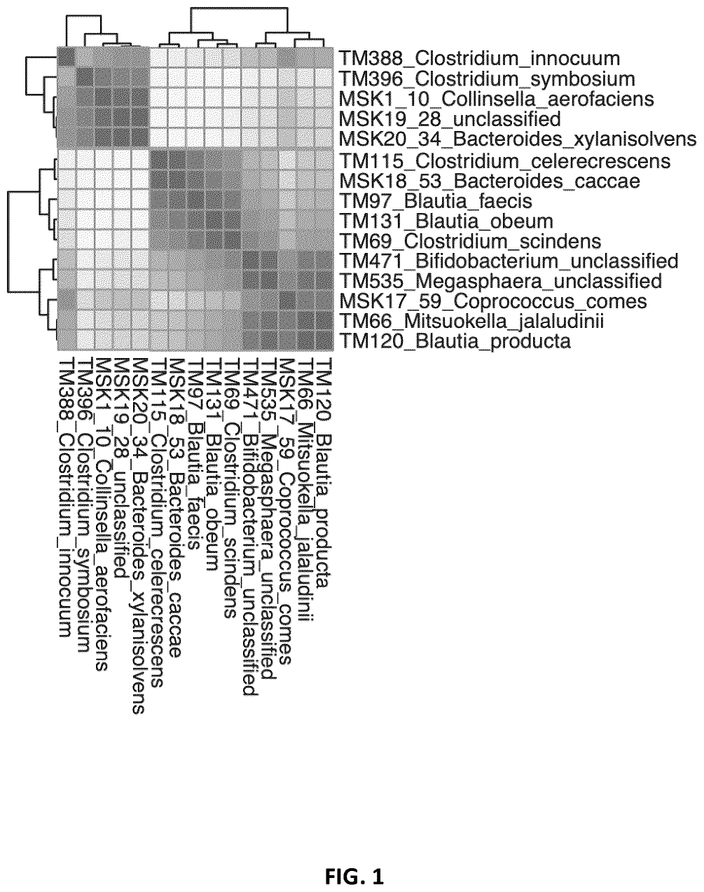

FIG. 1 shows various strains with strain-strain covariance scores that characterize the relationship between a pair of bacterial strains in relation to ability to suppress K. pneumoniae. As can be seen, the strain-strain covariance scores are represented as a matrix of numerical values. Table 1 provides a description of the strains in FIG. 1, including the identity of the strain as provided in FIG. 1, the NCBI link associated with the strain, the strain name, and GenBank Accession Number.

TABLE 1
Non-limiting strains

| | | | | GenBank |
|---|---|---|---|---|
| | | | | Accession |
| No. | Identifier in FIG. 1 | NCBI Link | Strain Name | Number |
| 1 | TM388_Clostridium_innocuum | ncbi.nlm.nih.gov/nuccore/ | Clostridium | JAHPYH000000000.1 |
| | | JAHPYH000000000 | innocuum strain | |
| | | | MSK.7.7 | |
| 2 | TM396_Clostridium_symbosium | ncbi.nlm.nih.gov/nuccore/ | Clostridium | JAAISL000000000.1 |
| | | JAAISL000000000 | symbiosum strain | |
| | | | MSK.7.21 | |
| 3 | MSK1_10_Collinsella_aerofaciens | ncbi.nlm.nih.gov/nuccore/ | Collinsella | JAHONB010000000.1 |
| | | JAHONB000000000 | aerofaciens strain | |
| | | | MSK.1.10 | |
| 4 | MSK19_28_Escherichia_coli | ncbi.nlm.nih.gov/nuccore/ | Escherichia coli | JAJCGG010000000.1 |
| | | JAJCGG000000000 | strain MSK.19.28 | |
| 5 | MSK20_34_Bacteroides_xylanisolvens | ncbi.nlm.nih.gov/nuccore/ | Bacteroides | JAHOFO000000000.1 |
| | | JAHOFO000000000 | xylanisolvens | |
| | | | strain MSK.20.34 | |
| 6 | TM115_Clostridium_celerecrescens | ncbi.nlm.nih.gov/nuccore/ | Lacrimispora | JAAWUT000000000.1 |
| | | JAAWUT000000000 | celerecrescens | |
| | | | strain MSK.15.50 | |
| 7 | MSK18_53_Bacteroides_caccae | ncbi.nlm.nih.gov/nuccore/ | Bacteroides | JAHOHY000000000.1 |
| | | JAHOHY000000000 | caccae strain | |
| | | | MSK.18.53 | |
| 8 | TM97_Blautia_faecis | ncbi.nlm.nih.gov/nuccore/ | Blautia faecis | JAAITS000000000.1 |
| | | JAAITS000000000 | strain MSK.17.74 | |
| 9 | TM131_Blautia_obeum | ncbi.nlm.nih.gov/nuccore/ | Blautia obeum | JAAIPT000000000.1 |
| | | JAAIPT000000000 | strain MSK.13.37 | |
| 10 | TM69_Clostridium_scindens | ncbi.nlm.nih.gov/nuccore/ | Clostridium | JAAISN000000000.1 |
| | | JAAISN000000000 | scindens strain | |
| | | | MSK.5.24 | |
| 11 | TM471_Bifidobacterium_unclassified | ncbi.nlm.nih.gov/nuccore/ | Bifidobacterium | JAHOMM000000000.1 |
| | | JAHOMM000000000 | sp. MSK.13.1 | |
| 12 | TM535_Megasphaera_unclassified | ncbi.nlm.nih.gov/nuccore/ | Megasphaera | JAJBQH000000000.1 |
| | | JAJBQH000000000 | massiliensis | |
| | | | strain MSK.20.21 | |
| 13 | MSK17_59_Coprococcus_comes | ncbi.nlm.nih.gov/nuccore/ | Coprococcus | JAAIMB000000000.1 |
| | | JAAIMB000000000 | comes strain | |
| | | | MSK.17.59 | |
| 14 | TM66_Mitsuokella_jalaludinii | ncbi.nlm.nih.gov/nuccore/ | Mitsuokella | JAJBPZ000000000.1 |
| | | JAJBPZ000000000 | jalaludinii strain | |
| | | | MSK.21.47 | |
| 15 | TM120_Blautia_producta | ncbi.nlm.nih.gov/nuccore/ | Blautia producta | JAAWUJ000000000.1 |
| | | JAAWUJ000000000 | strain MSK.4.17 | |

In some aspects, strains comprise sequences having at least 90% (e.g., 91%, 92%, 93%, 94%, 95%, 96%, 97%, 98%, 99%, 99.5%, or 99.9%) sequence identity to any of the sequences identified in Table 1. In some aspects, strains comprise sequences having 99.5% sequence identity to any of the sequences identified in Table 1. In some aspects, strains comprise sequences having 99.9% sequence identity to any of the sequences identified in Table 1. In some aspects, strains comprise any of the sequences identified in Table 1.

The recitations “sequence identity,” “percent identity,” or for example, “comprising a sequence 90% identical to” or “having at least 90% sequence identity to,” as used herein, refer to the extent that sequences are identical on an amino acid-by-amino acid basis, or a nucleotide-by-nucleotide basis, or over a window of comparison. Thus, a “percentage of sequence identity” can be calculated by comparing two optimally aligned sequences (e.g., nucleic acid) over the window of comparison, determining the number of positions at which the identical nucleic acid base (e.g., A, T, C, G, U) or the identical amino acid residue (e.g., alanine (Ala), proline (Pro), serine (Ser), threonine (Thr), glycine (Gly), valine (Val), leucine (Leu), isoleucine (Ile), phenylalanine (Phe), tyrosine (Tyr), tryptophan (Trp), lysine (Lys), arginine (Arg), histidine (His), aspartic acid (Asp), glutamic acid (Glu), asparagine (Asn), glutamine (Gln), cysteine (Cys), and methionine (Met)) occurs in both sequences to yield the number of matched positions, dividing the number of matched positions by the total number of positions in the window of comparison (i.e., the window size), and multiplying the result by 100 to yield the percentage of sequence identity.

Calculations of sequence similarity or sequence identity between sequences (the terms are used interchangeably herein) can be performed as follows. To determine the percent identity of two amino acid sequences, or of two nucleic acid sequences, the sequences can be aligned for optimal comparison purposes (e.g., gaps can be introduced in one or both of a first and a second amino acid or nucleic acid sequence for optimal alignment and non-homologous sequences can be disregarded for comparison purposes. The comparison of sequences and determination of percent identity between two sequences can be accomplished using a mathematical algorithm. In some aspects, the percent identity between two amino acid sequences is determined using the Needleman and Wunsch, J. Mol. Biol., 1970; 48: 444-453) algorithm, which has been incorporated into the GAP program in the GCG software package, using either a BLOSUM 62 matrix or a PAM250 matrix, and a gap weight of 16, 14, 12, 10, 8, 6, or 4 and a length weight of 1, 2, 3, 4, 5, or 6. 0), using a PAM120 weight residue table, a gap length penalty of 12 and a gap penalty of 4.

Identification of a bacterial strain can use 16S rRNA gene analysis (see, for example, Janda et al., “16S rRNA gene sequencing for bacterial identification in the diagnostic laboratory: pluses, perils, and pitfalls,” J. Clin. Microbiol., 2007; 45(9): 2761-2764), average nucleotide identity (ANI) analysis (see, for example, Konstantinidis et al., “Genomic insights that advance the species definition for prokaryotes,” Proc. Nat'l Acad. Sci. USA, 2005; 102(7):2567-2572), average amino acid identity (AAI) analysis (see, for example, Konstantinidis et al., “Towards a genome-based taxonomy for prokaryotes,” J. Bacteriol. 2005; 187(18):6258-6264), DNA-DNA hybridization (DDH) analysis (see, for example, Achtman et al., “Microbial diversity and the genetic nature of microbial species,” Nat. Rev. Microbiol., 2008; 6(6): 431-440), in silico DDH analysis (see, for example, Meier-Kolthoff et al., “Genome sequence-based species delimitation with confidence intervals and improved distance functions,” BMC Bioinformat. 2013; 14(1): article no. 60), and multi-locus sequence analysis (MLSA) (see, for example, Ciccarelli et al., “Toward automatic reconstruction of a highly resolved tree of life,” Science 2006; 311(5765): 1283-1287). In some aspects, identification of a bacterial strain can use one more methods selected from the group consisting of 16S rRNA gene analysis, average nucleotide identity analysis (ANI), average amino acid identity analysis (AAI), DNA-DNA hybridization analysis (DDH), in silico DDH analysis, and multi-locus sequence analysis (MLSA).

In some aspects, identification of a bacterial strain can use a method of 16S rRNA gene analysis. Briefly, 16s rRNA sequencing analysis compared the percentage sequence identity between bacteria. 16S ribosomal RNA (or 16S rRNA) is the RNA component of the 30S subunit of a prokaryotic ribosome. The bacterial 16S gene contains nine hypervariable regions (V1-V9), ranging from about 30 to 100 base pairs long, that are involved in the secondary structure of the small ribosomal subunit. Between the hypervariable regions are conserved regions. Sequencing of the full-length 16s rRNA or subsections (e.g., V1-V2, V1-V3, V3-V4, V4, V4-V5, V6-V8, and V7-V9) can be performed. 16s rRNA results in identified operational taxonomic unity (OTUs) at a threshold of 97% sequence identity. Amplicon sequence variants (ASVs) or zero-radius OTUs (zOTUs) have been suggested as alternatives to OTUs, as these approaches correct for sequencing errors by different denoising approaches. See, for example, Callahan et al., “DADA2: High-resolution sample inference from Illumina amplicon data,” Nat. Methods, 2016; 13:581-583 and/or Edgar, “Updating the 97% identity threshold for 16S ribosomal RNA OTUs,” Bioinformatics 2018; 34: 2371-2375. Exemplary primer sets for 16s rRNA sequencing in which a forward primer (denoted by F) and reverse primer (denoted by R) are used to amplify the 16s rRNA or portion thereof are provided in Table 2.

TABLE 2
16s rRNA primers

| Primer name | Sequence (5′-3′) | SEQ ID NO: |
|---|---|---|
| 8F | AGAGTTTGATCCTGGCTCAG | 1 |
| 27F | AGAGTTTGATCMTGGCTCAG | 2 |
| 336R | ACTGCTGCSYCCCGTAGGAGTCT | 3 |
| 337F | GACTCCTACGGGAGGCWGCAG | 4 |
| 518R | GTATTACCGCGGCTGCTGG | 5 |
| 533F | GTGCCAGCMGCCGCGGTAA | 6 |
| 785F | GGATTAGATACCCTGGTA | 7 |
| 806R | GGACTACVSGGGTATCTAAT | 8 |
| 907R | CCGTCAATTCCTTTRAGTTT | 9 |
| 928F | TAAAACTYAAAKGAATTGACGGG | 10 |
| 1100F | YAACGAGCGCAACCC | 11 |
| 1100R | GGGTTGCGCTCGTTG | 12 |
| U1492R | GGTTACCTTGTTACGACTT | 13 |
| 1492R | CGGTTACCTTGTTACGACTT | 14 |

The genes coding for it are referred to as 16S rRNA genes and are used in reconstructing phylogenies (e.g., taxonomic relationships), due to the slow rates of evolution of this region of the gene. In some aspects, bacteria of the same strain have at least 98% 16s rRNA sequence identity (e.g., 98%, 99%, 99.5%, 99.6%, 99.7%, 99.8%, 99.9%, or 100%).

In some aspects, identification of a bacterial strain can use a method of average nucleotide identity (ANI) analysis. In some aspects, bacteria of the same strain have at least 96% ANI (e.g., from 96% to less than 100%, from 97% to 100%, or from 98% to 100%).

Briefly, ANI determined the average nucleotide identity across all conserved predicted protein-coding genes between bacteria of the same species. Conserved genes are identified in a pairwise comparison amongst all bacterial strains of the same species. Bacteria of the same species have at least 94% ANI. Bacteria of the same strain have at least 96% ANI (e.g., 96% ANI, 97% ANI, or 98% ANI). In some aspects, ANI is calculated with an algorithm (e.g., a BLAST algorithm, such as the ANIb algorithm or the OrthoANIb algorithm). Examples of ANI algorithms can be found, for example, in Yoon et al., “A large-scale evaluation of algorithms to calculate average nucleotide identity,” Antonie Van Leeuwenhoek, 2017; 110(10): 1281-1286.

### Methods of Use

The present document encompasses methods of using a microbial consortium. For example and without limitation, the microbial consortia herein can be useful for treating a disease. In some aspects, the microbial consortia herein can be useful for modulating one or more markers in a subject (e.g., a marker such as a short chain fatty acid and/or an amino acid).

Methods can include use of a composition (e.g., including a microbial consortium, such as any herein including one or more strains) to modulate a short chain fatty acid and/or an amino acid, as compared to a control when the composition is not used. Modulating can include increasing or decreasing (e.g., enhancing or inhibiting) a response. In some aspects, an increase in response can include an increase by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 350%, 400%, 450%, 500%, 550%, 600%, 650%, 700%, 750%, 800%, 850%, 900%, 950%, 1000%, or greater than 1000%, as compared to a control in which a composition is not employed. In some aspects, a decrease in response can include a decrease by about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or even 100%, as compared to a control in which a composition is not employed.

In some aspects, use of a composition (e.g., including a microbial consortium, such as any described herein including one or more strains) can be characterized by an increase in short chain fatty acids (e.g., C₄-C₆ chain fatty acids), as compared to a control when the composition is not used. Such an increase can include an increase by about 1%, 2%, 3%, 4%, 5%, 6%, 7%, 8%, 9%, 10%, 15%, 20%, 25%, 30%, 35%, 40%, 45%, or more of a concentration of one or more short chain fatty acids (e.g., C₄-C₆ chain fatty acids), as compared to a control when the composition is not used. Non-limiting examples of short chain fatty acids include RCO₂H or an anionic form thereof or an ester form thereof or a salt form thereof, in which R is a C₁-C₆, C₂-C₆, C₃-C₆, C₄-C₆, or C₅-C₆ aliphatic group (e.g., alkyl group, including linear and branched forms thereof). In some aspects, R can be unsubstituted or substituted (e.g., with amino). Non-limiting examples of short chain fatty acids include formic acid, acetic acid, propionic acid, butyric acid, isobutyric acid, valeric acid, 5-amino valeric acid, isovaleric acid, 2-methylbutyric acid, hexanoic acid, as well as an anionic form thereof or an ester form thereof or a salt form thereof.

In some aspects, use of a composition (e.g., including a microbial consortium, such as any herein including one or more strains) can be characterized by a decrease in amino acids, as compared to a control when the composition is not used. Such an decrease can include a decrease about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, or more of a concentration of one or more amino acids, as compared to a control when the composition is not used. Non-limiting examples of amino acids include any herein, including phenylalanine (Phe) and alanine (Ala).

Methods can include a method for treating or prophylactically treating pneumonia. In some aspects, the method includes administering a therapeutically effective amount of a composition (e.g., any described herein) to a subject, thereby treating or prophylactically treating pneumonia. In some aspects, the pneumonia is community-acquired pneumonia. In some aspects, the pneumonia is caused by Enterobacteriaceae (e.g., Klebsiella, Klebsiella pneumoniae, or hypervirulent Klebsiella pneumoniae), drug resistant Enterobacteriaceae, or multi-drug resistant Enterobacteriaceae (e.g., carbapenem-resistant Enterobacteriaceae or carbapenem-resistant Klebsiella pneumoniae, such as K. pneumoniae strain MH258).

Methods can include a method for treating or prophylactically treating an infection, the method comprising administering a therapeutically effective amount of a composition to a subject, thereby treating or prophylactically treating infection. In some aspects, the infection is caused by Enterobacteriaceae (e.g., Klebsiella, Klebsiella pneumoniae, or hypervirulent Klebsiella pneumoniae), drug resistant Enterobacteriaceae, or multi-drug resistant Enterobacteriaceae (e.g., carbapenem-resistant Enterobacteriaceae or carbapenem-resistant Klebsiella pneumoniae, such as K. pneumoniae strain MH258).

Any of the methods herein can further include use of one or more antibiotics. For example and without limitation, a composition including one or more strains (e.g., any described herein) can be administered with one or more antibiotics. In another non-limiting example, the composition can be administered before or after administration with one or more antibiotics. The composition and the antibiotic(s) can be provided together (e.g., formulated separately but administered at the same time or formulated together) or separately. Furthermore, the treatment regimen (e.g., dosing time, dosage, dosage frequency, length of dosage regimen, etc.) can be the same or different.

Non-limiting examples of antibiotics include, e.g., amoxicillin, ampicillin, azithromycin, ciprofloxacin, clarithromycin, colistin, doxycycline, gentamycin, kanamycin, levofloxacin, metronidazole, neomycin, vancomycin, and the like.

Yet other non-limiting examples of antibiotics include, e.g., aminoglycosides (e.g., amikacin, apramycin, arbekacin, astromicin, bekanamycin, dibekacin, dihydrostreptomycin, framycetin, gentamicin, hygromycin B, isepamicin, kanamycin, micronomicin, neomycin, netilmicin, nourseothricin, paromomycin, plazomicin, rhodostreptomycin, ribostamycin, sisomicin, spectinomycin, streptomycin, tobramycin, totomycin, verdamicin, and the like); antifolates (e.g., acediasulfone, dapsone, iclaprim, ormetoprim, pyrimethamine, solasulfone, sulfadiazine, sulfadimethoxine, sulfadoxine, sulfamethoxazole, sulfoxone, tetroxoprim, trimethoprim, and the like) optionally in combinations, such as in ormetoprim/sulfadimethoxine, pyrimethamine/dapsone, pyrimethamine/sulfadoxine, trimethoprim/sulfamethoxazole, and the like; cephems, including cephalosporins (e.g., cefamandole, cefadroxil, cefaclor, cefalexin, cefamandole, cefazolin, cefepime, cefiderocol, cefixime, cefoperazone, cefotaxime, cefoxitin, cefpodoxime, cefprozil, ceftriaxone, ceftobiprole, cefuroxime, cephalexin, cephaloglycin, cephaloridine, cephalothin, cephapirin, cephradine, ceftriaxone, defotaxime, ceftazidime, cefepime, and the like); ketolides (e.g., telithromycin); macrolides (e.g., azithromycin, clarithromycin, erythromycin, troleandomycin, and the like); monobactams (e.g., aztreonam); nitro derivatives, such as nitroimidazoles or nitrofurans (e.g., furazolidone, metronidazole, nifuroxazide, nifurtoinol, nifurzide, nitrofurantoin, nitrofurazone, ornidazole, secnidazole, tinidazole, and the like); oxazolidinones (e.g., eperezolid, linezolid, posizolid, radezolid, ranbezolid, sutezolid, tedizolid, and the like); penams, including penicillins (e.g., amoxicillin, ampicillin, carbenicillin, cloxacillin, dicloxacillin, methicillin, nafcillin, oxacillin, phenethicillin, penicillin G, piperacillin, ticarcillin, and the like) optionally with a β-lactamase inhibitor (e.g., clavulanic acid, sulbactam, tazobactam, or any described herein), such as ampicillin/sulbactam, amoxicillin/clavulanate, piperacillin/tazobactam, and ticarcillin/clavulanate, and the like; penems or carbapenems (e.g., doripenem, ertapenem, imipenem, meropenem, and the like); polypeptide antibiotics (e.g., bacitracin, colistimethate, colistin, polymyxin B, telavancin, vancomycin, and the like); quinolones or fluoroquinolones (e.g., alatrofloxacin, besifloxacin, ciprofloxacin, clinafloxacin, delafloxacin, finafloxacin, garenoxacin, gatifloxacin, gemifloxacin, levofloxacin, moxifloxacin, nemonoxacin, norfloxacin, ofloxacin, ozenoxacin, prulifloxacin, sitafloxacin, tequin, trovafloxacin, and the like); rifamycins (e.g., rifampicin, rifabutin, rifapentine, rifaximin, rifalazil, and the like); tetracyclines (e.g., chlortetracycline, demeclocycline, doxycycline, methacycline, minocycline, oxytetracycline, rolitetracycline, sarecycline, tetracycline, tigecycline, and the like); and combinations of any of these.

In some aspects, one or more antibiotics are combined with one or more β-lactamase inhibitors (e.g., avibactam, clavulanic acid, durlobactam, relebactam, sulbactam, tazobactam, tebipenem, vaborbactam, and the like), such as in, e.g., ceftazidime/avibactam, amoxicillin/clavulanic acid, ticarcillin/clavulanic acid, imipenem/cilastatin/relebactam, ampicillin/sulbactam, cefoperazone/sulbactam, ceftolozane/tazobactam, piperacillin/tazobactam, meropenem/vaborbactam, and the like.

Any of the methods herein can further include use of one or more therapeutics. In some aspects, the therapeutic(s) is beneficial for use in a subject having or likely to have pneumonia or an infection (e.g., from K. pneumoniae). In some aspects, the one or more therapeutics is selected from the group of a β-lactamase inhibitor (e.g., avibactam, clavulanic acid, durlobactam, relebactam, sulbactam, tazobactam, tebipenem, vaborbactam, and the like), an amino acid (e.g., any described herein), an aminosalicylate (e.g., balsalazide, mesalamine, sulfasalazine, and the like), an analgesic (e.g., acetaminophen), an antifungal, an antihistamine, an anti-inflammatory agent (e.g., sulfasalazine or an NSAID, such as any described herein), an antimicrobial agent (e.g., an antibiotic, an antifungal, an antiviral, or an anti-parasitic agent), an anti-parasitic agent, an antiviral, a carbohydrate, a corticosteroid (e.g., such as a glucocorticoid (e.g., prednisone)), a cytokine, a hormone (e.g., epinephrine), an immunosuppressant (e.g., cyclosporin A, methotrexate, and the like), a nonsteroidal anti-inflammatory agent (an NSAID, e.g., aspirin, ibuprofen, or naproxen), a peptide, a prebiotic (e.g., one or more amino acids, peptides, proteins, complex carbohydrates, simple carbohydrates, vitamins, essential metals, essential minerals, buffering agents, or other nutritional components to maintain the microbial consortium, such as any described herein), or a combination of any of these. The composition and the therapeutic(s) can be provided together (e.g., formulated separately bur administered at the same time or formulated together) or separately. The composition and the therapeutic(s) can be used together (e.g., co-administered using same or different routes of administration) or separately (e.g., administered at differing times using same or different routes of administration). Furthermore, the treatment regimen (e.g., dosing time, dosage, dosage frequency, length of dosage regimen, etc.) can be the same or different.

In some aspects, the therapeutic (e.g., for use with one or more strains) is an antiviral agent. Non-limiting examples of antivirals include, e.g., abacavir, acyclovir, adefovir, amantadine, amprenavir, atazanavir, bictegravir, BI 224436, cabotegravir, cidofovir, cobicistat, darunavir, delavirdine, didanosine, docosanol, dolutegravir, efavirenz, elvitegravir, emtricitabine, enfuvirtide, etravirine, famciclovir, fosamprenavir, foscarnet, fomivirsen, ganciclovir, ibacitabine, indinavir, idoxuridine, lamivudine, lenacapavir, lopinavir, maraviroc, MK-2048, nelfinavir, nevirapine, oseltamivir, penciclovir, raltegravir, rilpivirine, rimantidine, ritonavir, saquinavir, stavudine, tenofovir, tipranavir, TMC-310911, trifluridine, valaciclovir, valganciclovir, vidarabine, zalcitabine, zanamivir, and zidovudine.

In some aspects, the therapeutic (e.g., for use with one or more strains) is an antifungal agent. Non-limiting examples of antifungals include, e.g., allylamines or benzylamines, such as butenafine, naftifine, terbinafine, and the like; echinocandins, such as anidulafungin, caspofungin, micafungin, and the like; imidazoles, such as bifonazole, butoconazole, clotrimazole, econazole, fenticonazole, isoconazole, ketoconazole, luliconazole, miconazole, omoconazole, oxiconazole, sertaconazole, sulconazole, tioconazole, and the like; polyenes, such as amphotericin B, candicidin, filipin, hamycin, natamycin, nystatin, rimocidin, and the like; thiazoles, such as abafungin and the like; triazoles, such as albaconazole, efinaconazole, epoxiconazole, fluconazole, isavuconazole, itraconazole, posaconazole, propiconazole, ravuconazole, terconazole, voriconazole, and the like; triterpenoids, such as ibrexafungerp and the like; and others, such as benzoic acid, ciclopirox, flucytosine (5-fluorocytosine), griseofulvin, haloprogin, miltefosine, polygodial, tolnaftate, undecylenic acid, and the like.

In some aspects, the therapeutic (e.g., for use with one or more strains) is a prebiotic. Non-limiting examples of prebiotics include, e.g., amino acids, such as cysteine or any described herein; elements or minerals, such as boron, bromine, calcium, chloride, cobalt, copper, iron, magnesium, manganese, molybdenum, nickel, nitrogen, phosphorous, potassium, sodium, strontium, sulfur, vanadium, zinc, and the like; polyphenols (e.g., catechin, ellagitannin, isoflavone, flavonol, flavanone, anthocyanin, lignin, and the like; vitamins, such as pantothenate, thiamine, riboflavin, niacin, pyridoxol, biotin, folate, 4-aminobenzoate, cobinamide, a cobamide (e.g., phenyolyl cobamide, 5-methylbenzimidazolyl cobamide), or cobalamin, or salts or derivatives thereof), and the like; fructooligosaccharides, such as oligofructose or oligofructan; galactooligosaccharides; galactans; isomaltooligosaccharides; mannan oligosaccharides; oligofructose-enriched inulin; oligofructose; oligodextrose; trans-galactooligosaccharides; xylooligosaccharides; sugar alcohols, such as lactitol, maltitol, sorbitol, and the like; monosaccharides, such as fructose, galactose, glucose, tagatose, and the like; disaccharides, such as lactulose, paratinose (or isomaltulose), and the like; trisaccharides, such as raffinose, lactosucrose, and the like; tetrasaccharides, such as stachyose; polysaccharides, such as glycogen, inulin, mannan, pectin, beta-glucan, and the like; chitosan oligosaccharides (chioses); genito-oligosaccharides; glucooligosaccharides; soy- and pectin-oligosaccharides; pecticoligosaccharides; palatinose polycondensates and oligosaccharides; soybean oligosaccharides (e.g., soyoligosaccharides); carbohydrate based gums, such as carrageen, gellan, guar, konjac, lactulose, psyllium, and the like; or mixtures thereof.

The therapeutic agents of the disclosure may be administered by the same route of administration or by different routes of administration. In some aspects, the therapeutic composition is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the patient, the patient's clinical history and response to the treatment, and the discretion of the attending physician.

In some aspects, the first therapeutic composition, which may comprise one or more of the bacterial composition disclosed herein, is administered at a dose of between 1×10⁵ to 9×10⁸ CFUs, or any range derivable therein. In some aspects, the first therapeutic composition is administered at a dose of at least, atmost, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria.

In some aspects, a single dose of the second therapeutic composition, which may comprise a metabolite, antibiotic, or other therapy disclosed herein. In some aspects, multiple doses of the second therapeutic composition are administered. In some aspects, the second therapeutic composition is administered at a dose of 1 μg/kg to 1 mg/kg, or any range derivable therein, or between 1 mg/kg and 100 mg/kg, or any range derivable therein. In some aspects, the second therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, μg/kg or mg/kg.

### Compositions

The present document encompasses compositions, which can include any useful combination of strains provided herein. In some aspects, the composition includes one or more pharmaceutically acceptable excipients.

In some aspects, the composition comprises one, more than one, or all of the bacteria of Block 1. In some aspects, the composition comprises one, more than one, or all of the bacteria of Block 2. In some aspects, the composition comprises one, more than one, or all of the bacteria of Block 3. In some aspects, the composition comprises one, more than one, or all of the bacteria of Block 4. In some aspects, the composition comprises one, more than one, or all of the bacteria of Block 5. In some aspects, the composition comprises one, more than one, or all of the bacteria of Block 1 and Block 5 (which may, in some aspects, be referred to as SynCom15). In some aspects, the composition comprises one, more than one, or all of the bacteria of Block 1, Block 2, Block 3, Block 4, Block 5, or any combination thereof.

In some aspects, Block 1 comprises, consists of, or consists essentially of: Collinsella aerofaciens, Escherichia coli, Bacteroides xylanisolvens, Clostridium symbiosum, and Clostridium innocuum.

In some aspects, Block 2 comprises, consists of, or consists essentially of: Ruminococcus faecis, Bacteroides eggerthii, Lactococcus lactis, Phocaeicola massiliensis, Bifidobacterium breve, Dorea formicigenerans, and Veillonella ratti.

In some aspects, Block 3 comprises, consists of, or consists essentially of: Blautia luti, Bifidobacterium pseudocatenulatum, Blautia hansenii, Ruminococcus lactaris, Acidaminococcus intestine, Enterocloster clostridioformis, Bacteroides uniformis, Megasphaera elsdenii, Bacteroides thetaiotaomicron, and Megamonas funiformis.

In some aspects, Block 4 comprises, consists of, or consists essentially of: Odoribacter splanchnicus, Parabacteroides merdae, Sellimonas intestinalis, Clostridium cochlearium, Lachnospiraceae sp., Erysipelatoclostridium ramosum, Blautia wexlerae, Bacteroides cellulosilyticus, Phocaeicola dorei, Prevotella stercorea, Bacteroides ovatus, Fusicatenibacter saccharivorans, Bifidobacterium adolescentis, and Parabacteroides sp.

In some aspects, Block 5 comprises, consists of, or consists essentially of: Bifidobacterium sp., Blautia obeum, Lacrimispora celerecrescens, Coprococcus comes, Blautia faecis, Bacteroides caccae, Megasphaera massiliensis, Mitsuokella jalaludinii, Blautia producta, and Clostridium scindens.

In some aspects, SynCom15 (Block1+Block 5) comprises, consists of, or consists essentially of: Collinsella aerofaciens, Bifidobacterium sp., Blautia obeum, Lacrimispora celerecrescens, Coprococcus comes, Blautia faecis, Bacteroides caccae, Escherichia coli, Megasphaera massiliensis, Bacteroides xylanisolvens, Mitsuokella jalaludinii, Blautia producta, Clostridium scindens, Clostridium symbiosum, and Clostridium innocuum.

The composition can be provided in any useful form, such as a dosage form having a unit dose. The term “unit dose” when used in reference to a therapeutic composition refers to physically discrete units suitable as unitary dosage for the individual, each unit containing a predetermined quantity of active material calculated to produce the desired therapeutic effect in association with the required physiologically acceptable diluent, e.g., carrier or vehicle. Optimal dosing occurs over a duration of about two weeks or less.

Any useful dosage forms can be implemented. Such dosage forms can include, e.g., tablets, capsules, caplets, particulates, enemas, suppositories, solutions, suspensions, emulsions, dispersions, powders, viscous compositions (e.g., gels), foams, creams, oils, sprays, and the like.

Dosage forms, such as tablets, capsules, caplets, or particulates, may, if desired, be formulated so as to provide for controlled release of the therapeutic compositions, where controlled release may be sustained release, delayed release, or a combination thereof. Controlled release formulations are preferably sustained release, meaning gradual delivery of the therapeutic compositions over an extended time period. Generally, as will be appreciated by those of ordinary skill in the art, sustained release dosage forms are formulated by dispersing the live bacterial strains and other active agents/ingredients within a matrix of a gradually hydrolyzable material such as a hydrophilic polymer, or by coating a solid, drug-containing dosage form with such a material. Hydrophilic polymers useful for providing a sustained release coating or matrix include, by way of example: cellulosic polymers such as hydroxypropyl cellulose, hydroxyethyl cellulose, hydroxypropyl methyl cellulose, methyl cellulose, ethyl cellulose, cellulose acetate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate; and vinyl polymers and copolymers such as polyvinyl pyrrolidone, polyvinyl acetate, and ethylene-vinyl acetate copolymer.

In some aspects, the first therapeutic composition is administered at a dose of between 1×10⁵ to 9×10⁸ CFUs, or any range derivable therein. In some aspects, the first therapeutic composition is administered at a dose of at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria.

In some aspects, a single dose of the second therapeutic composition, which may comprise a metabolite, antibiotic, and/or immunosuppressant. In some aspects, multiple doses of the second therapeutic composition are administered. In some aspects, the second therapeutic composition is administered at a dose of 1 μg/kg to 1 mg/kg, or any range derivable therein, or between 1 mg/kg and 100 mg/kg, or any range derivable therein. In some aspects, the second therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or any range derivable therein, μg/kg or mg/kg.

### EXAMPLES

### Example 1: Tested “Blocks” of Microbial Consortia

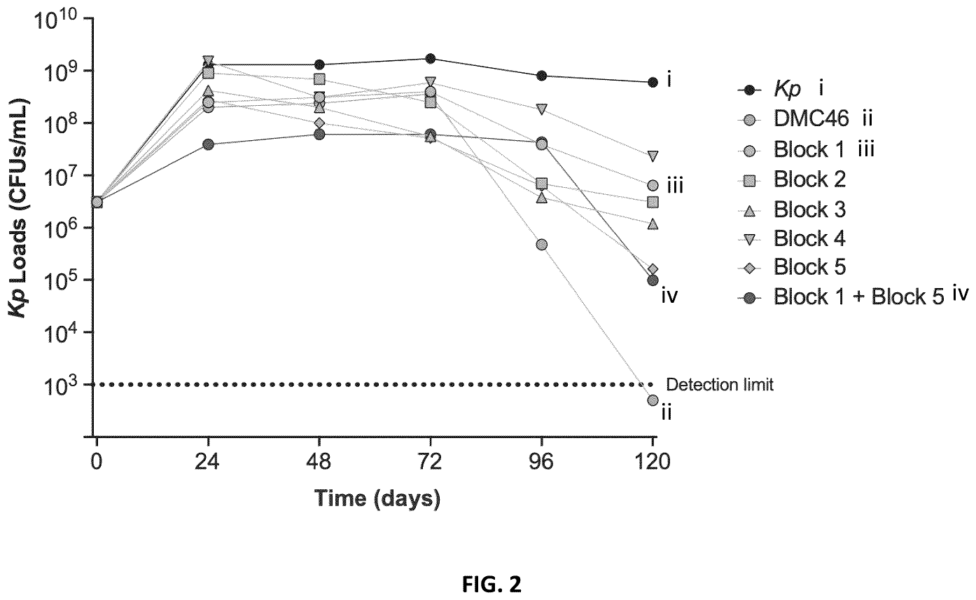

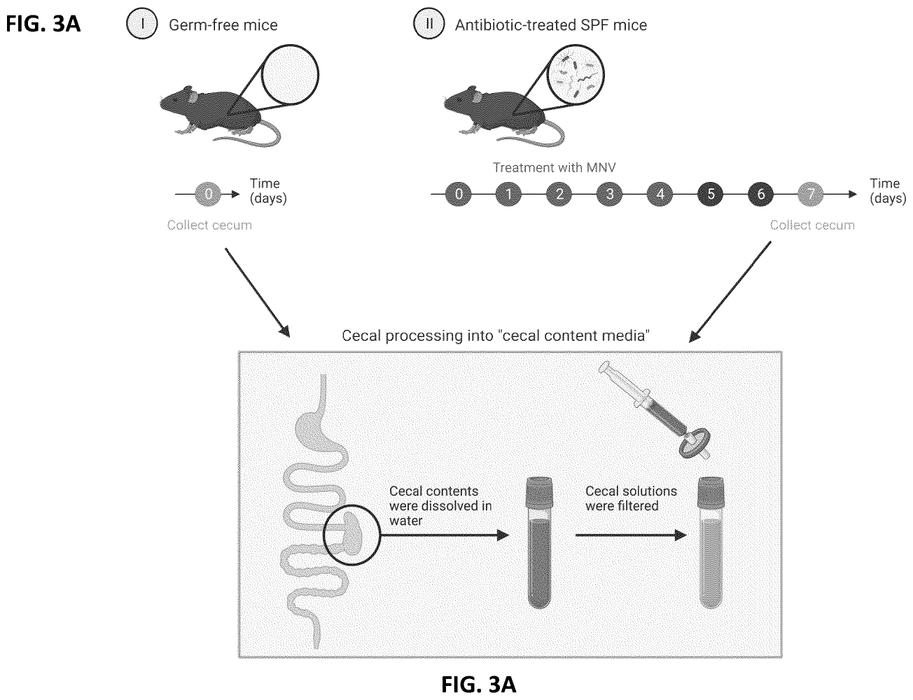

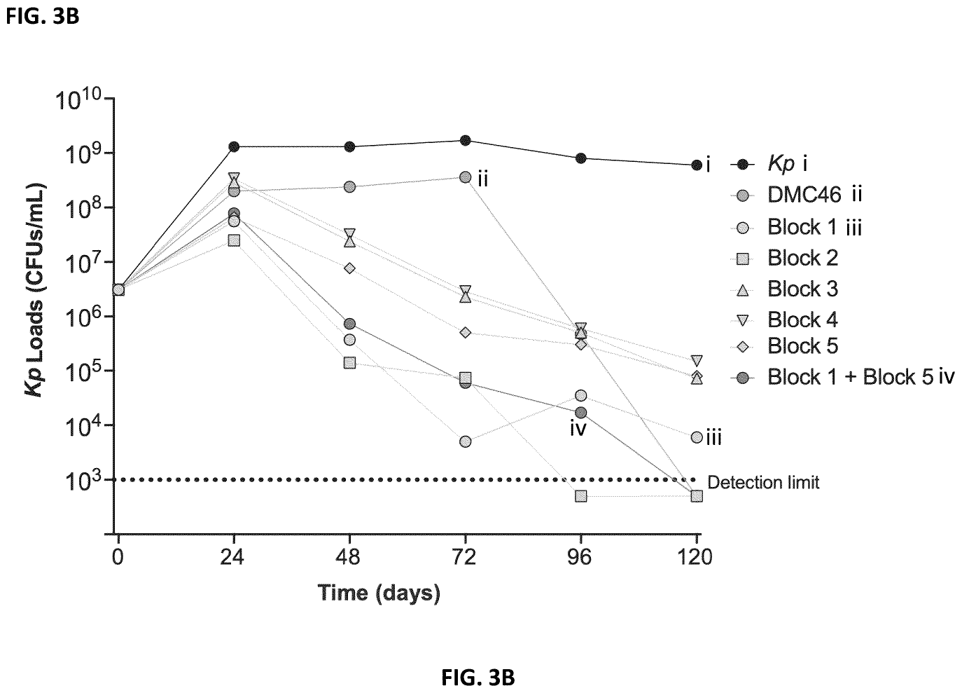

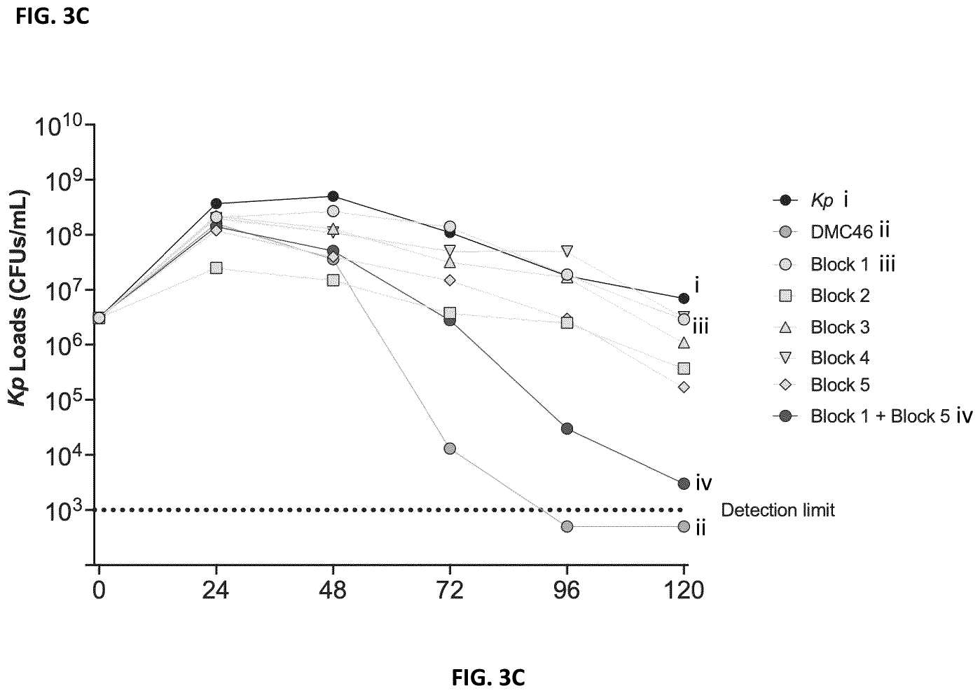

Designed microbial communities (DMCs) were tested for their ability to suppress Klebsiella pneumoniae (Kp). Tested DMCs included DMC46 (including 46 strains), Block 1, Block 2, Block 3, Block 4, Block 5, and Block 1+Block 5 (including 15 strains, also indicated as “synCom15”). Provided are testing results in BHIS media (FIG. 2) and in cecal extract media (FIG. 3A) from germ-free mice (FIG. 3B) or antibiotic-treated specific-pathogen free (SPF) mice (FIG. 3C). As can be seen, synCom15 was effective in all tested conditions.

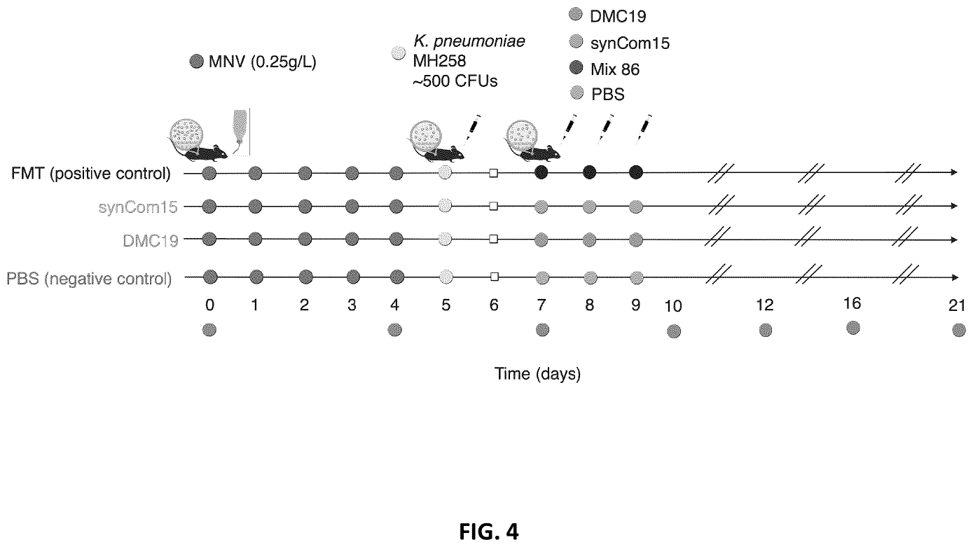

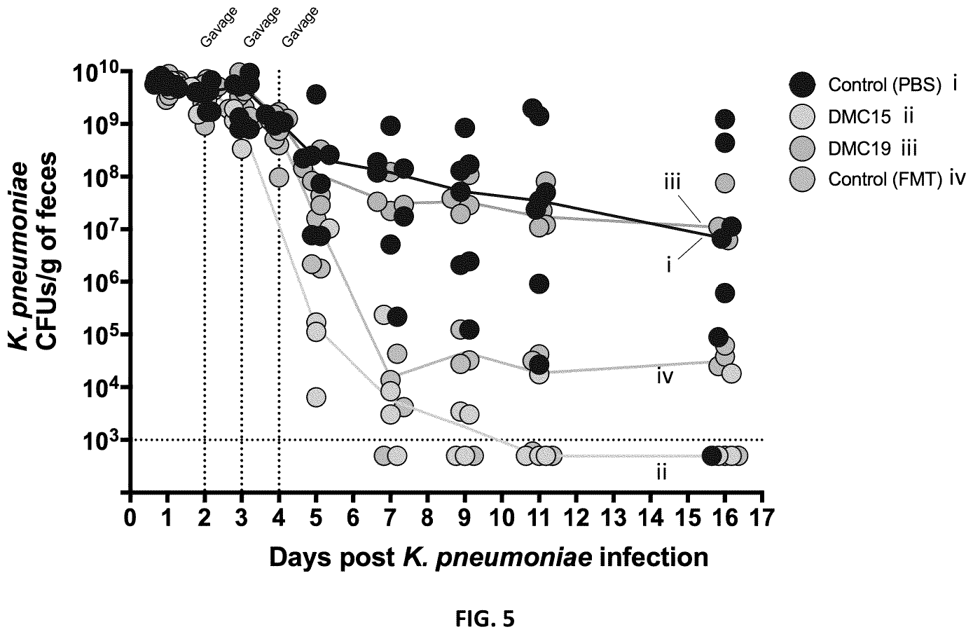

DMCs were also tested in a murine model for infection (FIG. 4). Specific pathogen free male mice (C57BL/6J) were singly housed, fed a normal mouse chow diet, given broad spectrum antibiotics for three days, then infected with K. pneumoniae (Kp), then given three doses (gavage) by both nasogastric tube orally and fecal enema of either saline (PBS), a fecal microbial transplant (FMT), or one of two designed microbial communities (DMCs): synCom15 and DMC19. The DMC19 consortium includes a collection of bacterial strains that share similar strain-strain covariance scores but that have relatively low impact scores. As seen in FIG. 5, experimental results indicated that synCom15 performed at least as well as Control (FMT), and outperforms Control (PBS) and DMC19 on the task of suppressing Kp bacteria.

### Example 2: Profile of Samples from synCom15 Treatments

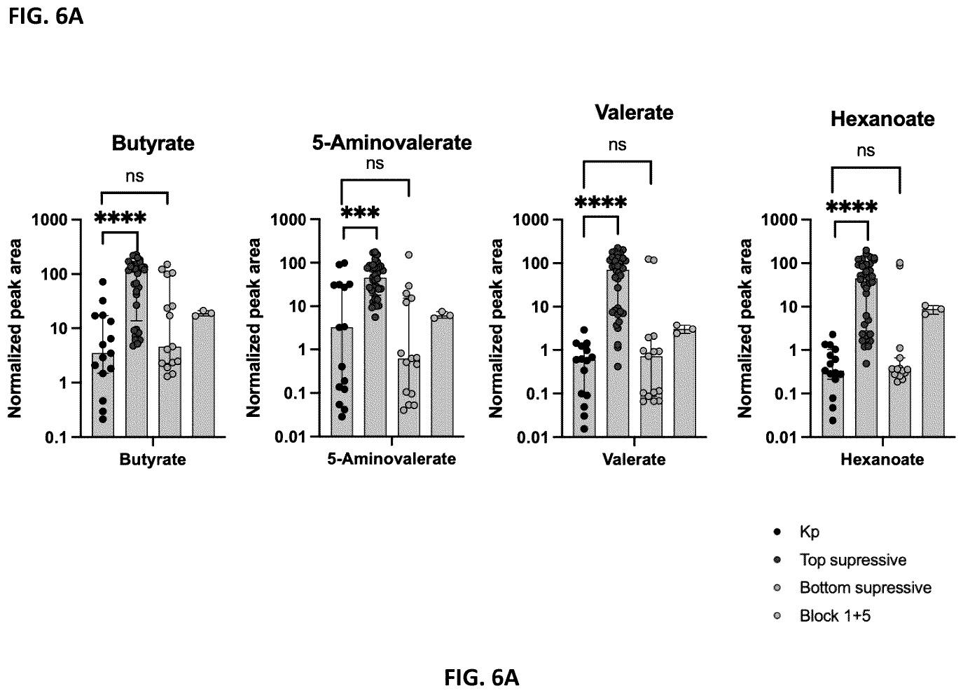

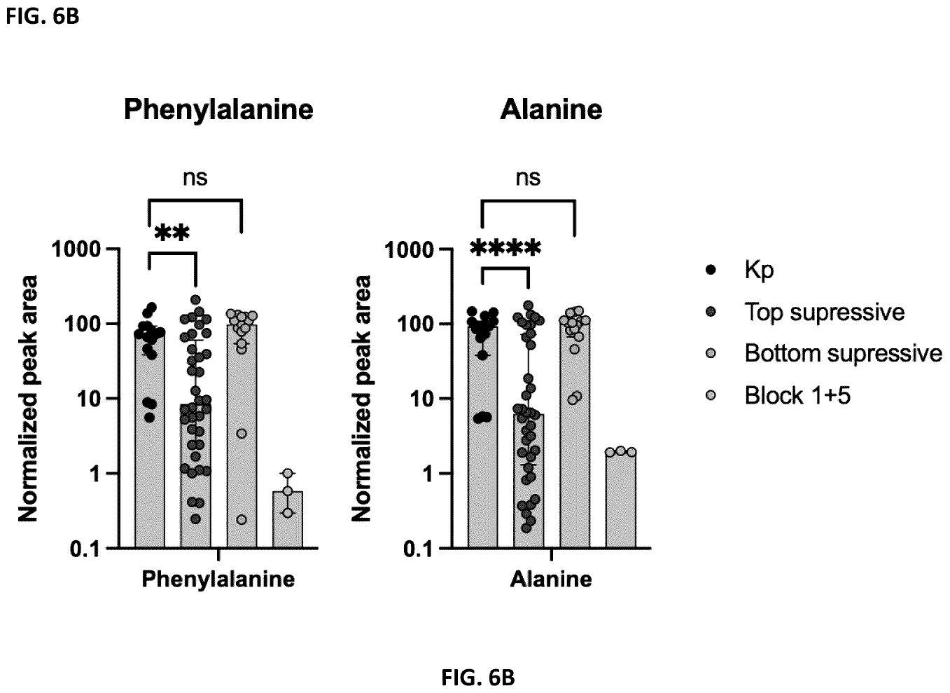

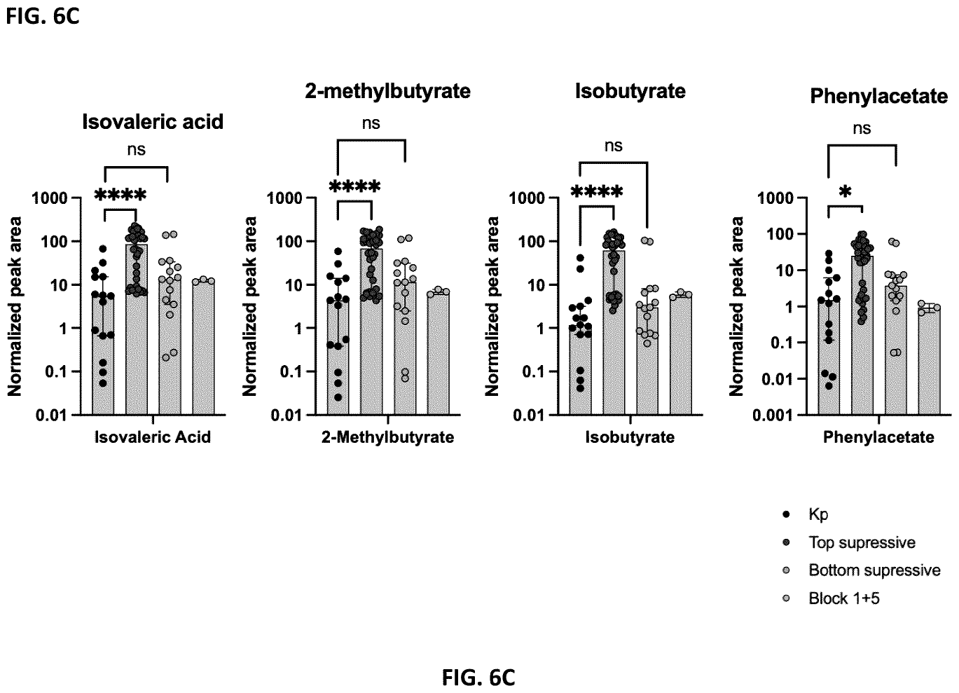

Further analysis was conducted to understand how synCom15 worked during treatment. As seen in FIGS. 6A-6C, the presence of fatty acids and amino acids was determined for samples obtained after infection with Kp, from top suppressive strains, bottom suppressive strains, and synCom15. Without wishing to be limited by a mechanism or theory, synCom15 appeared to produce short chain fatty acids (e.g., C₄-C₆ chain fatty acids) and deplete amino acids (e.g., phenylalanine and alanine), each of which (alone or together) may contribute to its efficacy.

### Example 3—Illustrative Methods

Creation and whole genome sequencing of strain bank: Fecal samples were obtained from 28 human donors that fell within the age range of 18 to 63 with a median age of 35. Donors were selected as those without a history of antibiotic use within a year prior to consent; and without known history of diabetes, colitis, autoimmune disease, cancer, pneumonia, dysentery, or cellulitis at time of consent. Institutions that approved protocols of fecal sample collection were Memorial Sloan Kettering (MSK) and the University of Chicago. Fresh fecal samples were immediately reduced in an anaerobic chamber upon collection and diluted and cultured on various growth media. Agar media types include any of the following: Columbia Blood Agar, Brain Heart Infusion+Yeast, Brain Heart Infusion+Mucin, Brain Heart Infusion+Yeast+Acetate or N-acetylglucosamine, reinforced Clostridial Agar, Peptone Yeast Glucose, Yeast Casitone Fatty Acids, and Defined media M5. Colonies were selected and grown to be sufficiently turbid, and 20% glycerol/PBS stocks were created and stored at −80° C.

Colonies were selected for whole-genome sequencing based on pyro-sequencing of the 16S region which provides a rough estimate of genus level designation. For each donor, only colonies that had a sequence identity threshold of less than 99% from CD-Hit (v. 4.8.1) were selected for whole-genome sequencing^{55,56}. Bacterial genomic DNA was extracted using QIAamp DNA Mini Kit (QIAGEN®) according to manufacturer's manual. The purified DNA was quantified using a Qubit 2.0 fluorometer. 1000 nanograms (ng) of each sample were prepared for sequencing using the QIAseq FX DNA Library Kit (QIAGEN®). The protocol was carried out for a targeted fragment size of 550 base pairs (bp). Sequencing was performed on the MiSeq or NextSeq platform (ILLUMINA®) with a paired-end (PE) kit in pools designed to provide 1-3 million PE reads per sample with read length of 250 or 150 bp.

Adapters were trimmed off with Trimmomatic (v0.39) with the following parameters: the leading and trailing 3 bp of the sequences were trimmed off, quality was controlled by a sliding window of 4, with an average quality score of 15 (default parameters of Trimmomatic)⁵⁷. Moreover, any read that was less than 50 bp long after trimming and quality control were discarded. The remaining high-quality reads were assembled into contigs using SPAdes (v3.15.4)⁵⁸.

Taxonomic classification of the assembled contigs was performed with the following methods: (a) Kraken2 (v2.1.2); (b) full/partial length 16S rRNA gene from each isolated colony's assembled contigs was extracted and inputted into BLASTn (v2.10.1+) to query against NCBI's RNA RefSeq database⁵⁹⁻⁶¹. The top five hits for each query were manually curated to determine an isolate's identity, with an identity and coverage cutoff of 95% for both; and (c) GTDB-Tk (v1.5.1)⁶². The final taxonomy was determined by the consensus of the three methods. Any colony that did not match initial pyro-sequencing taxonomy or lacked consensus was excluded from the commensal strain bank.

Construction of tree of bacterial genera across fecal microbiomes of healthy donors: From the metagenomic sequencing data of the fecal samples collected across healthy donors, bacterial genera present were identified by Metaphlan4⁶³. Names were then extracted and cross-referenced with NCBI taxonomy using the taxize application in R⁶⁴. The resulting tree was constructed based on NCBI taxonomic classification.

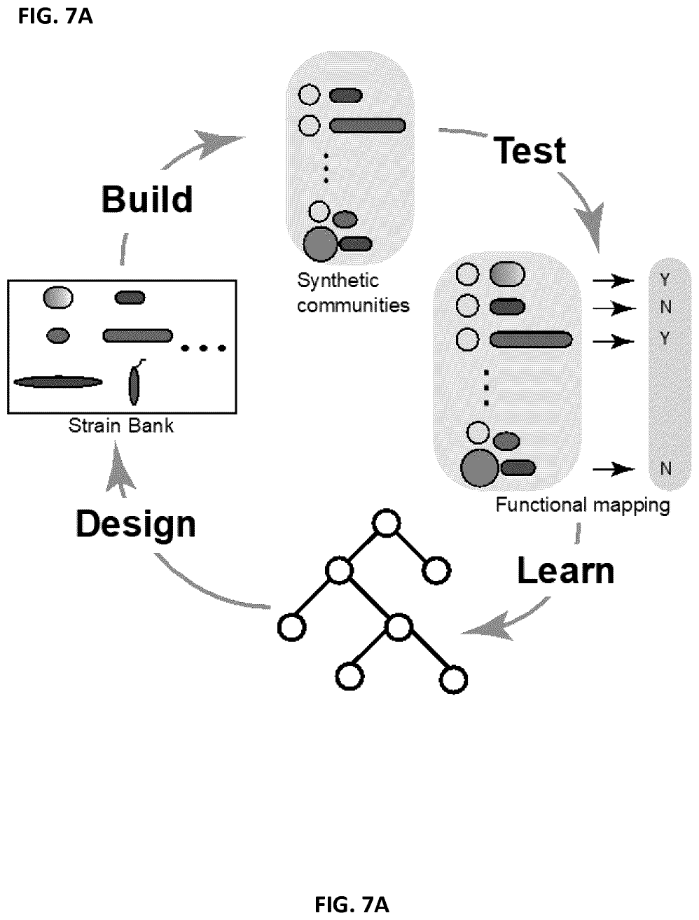

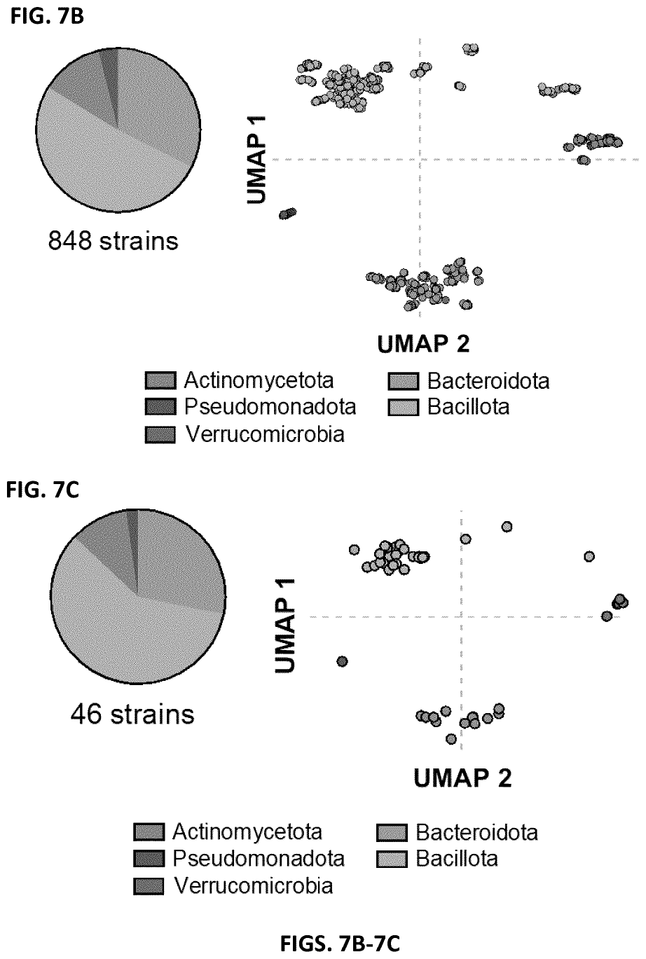

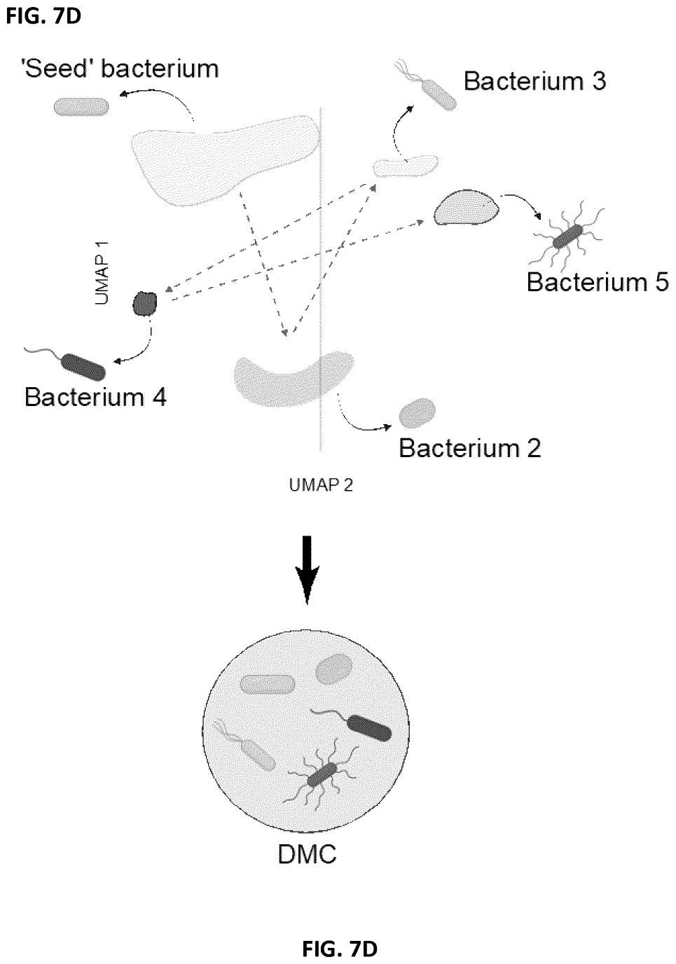

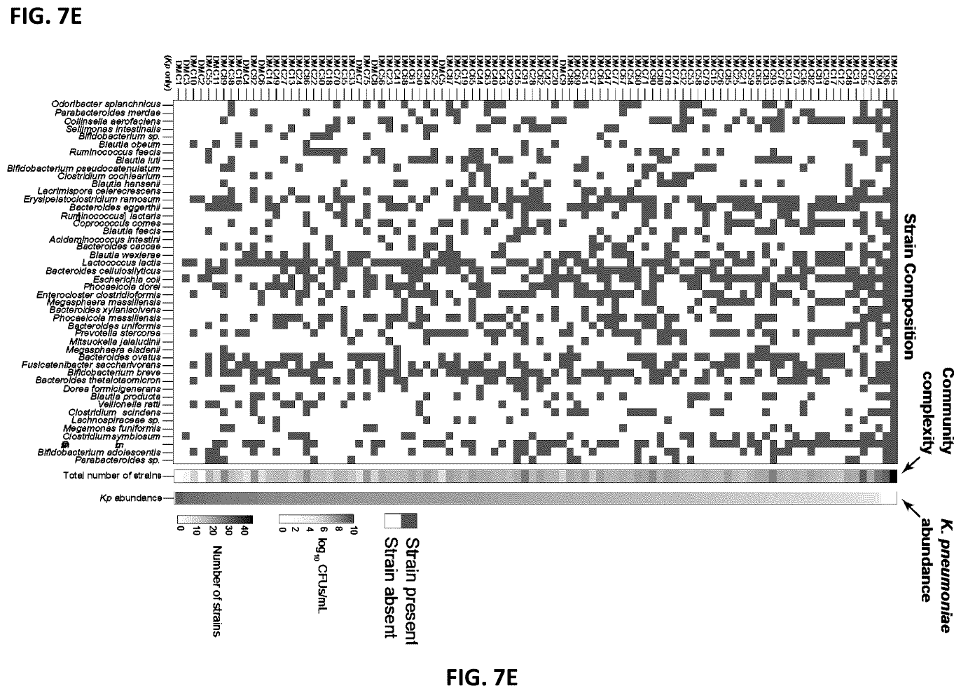

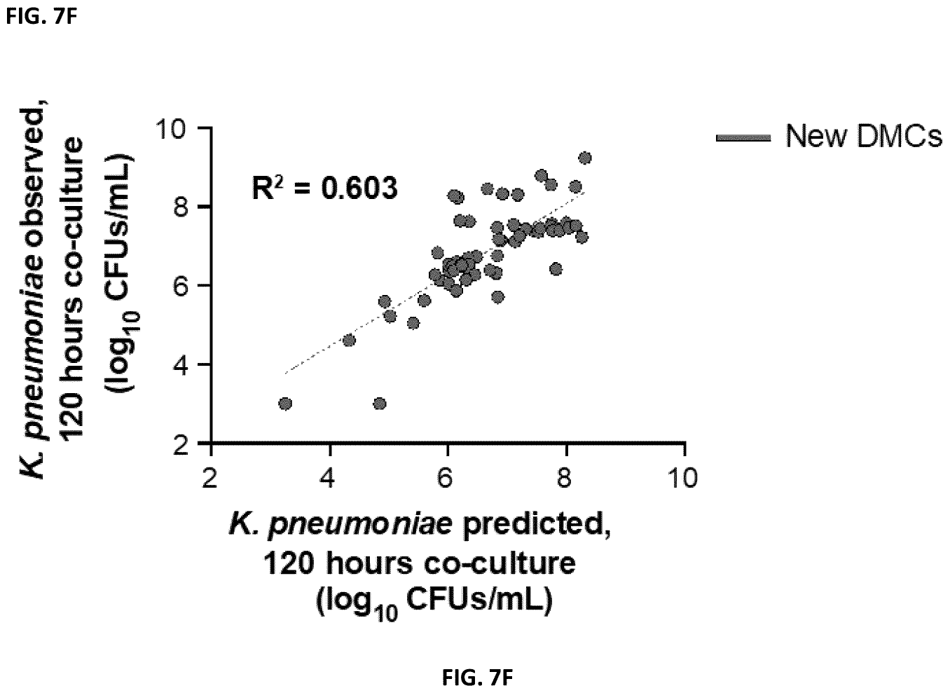

Construction of UMAP plot shown in FIGS. 7B and 7C: All gut commensal strains were annotated by their Prokka annotations and an alignment was created (848 rows comprising commensal strains, 150181 columns comprising Prokka annotated features). Each entry in the alignment is a ‘1’ or a ‘0’ indicating the presence or absence of a specific feature in a particular bacterial proteome.

Shotgun metagenomics of fecal samples from healthy human donors: Procedure for acquiring metagenomic data from fecal samples of healthy donors followed the same protocol as that described by Odenwald et al⁶⁵.

Design strategy for bacterial communities: To design a bacterial community comprised of N strains, the inventors perform the following steps using the UMAP plot based on bacterial genomes of 46 strains shown in FIG. 7C as the basis for their approach.

Step 1: Create 10,000 communities randomly of size N. The ensemble of all 10,000 communities of size N is represented as

$${C}_{size N} = \{ {c}_{1} , ... , {c}_{10 , 000} \} \qquad ( 1 )$$

Step 2: Each community, cᵢ, was defined by a set of N bacterial strains:

$${c}_{i} = \{ {s}_{1} , ... , {s}_{N} \} \qquad ( 2 )$$

where sⱼ is strain j in cᵢ. Compute all pairwise distances in the UMAP space for all strains in Cᵢ. For instance, the pairwise distance between strain 1 and 2 was:

$${pd}_{1 , 2} = dist ( {s}_{1} , {s}_{2} ) \qquad ( 3 )$$

where “dist” is the function that computes the distance between s₁ and s₂ in the UMAP space. The inventors defined the distribution of all pairwise distances for cᵢ as:

$${PD}_{i} = \{ {pd}_{1 , 2} , {pd}_{1 , 3} , ... , {pd}_{N - 1 , N} \} \qquad ( 4 )$$

Step 3: Order PDᵢ for a given cᵢ from largest to smallest values, then compute the mean pairwise distance across the lower 30% of values comprising PDᵢ. The inventors termed this value the “mean adjusted dispersal.”

Step 4: Compute the mean adjusted dispersal for all communities in C_{size N}.

Step 5: Identify the community within the 10,000 communities comprising C_{size N} with the maximum mean adjusted dispersal. This community was the designed community comprising N strains.

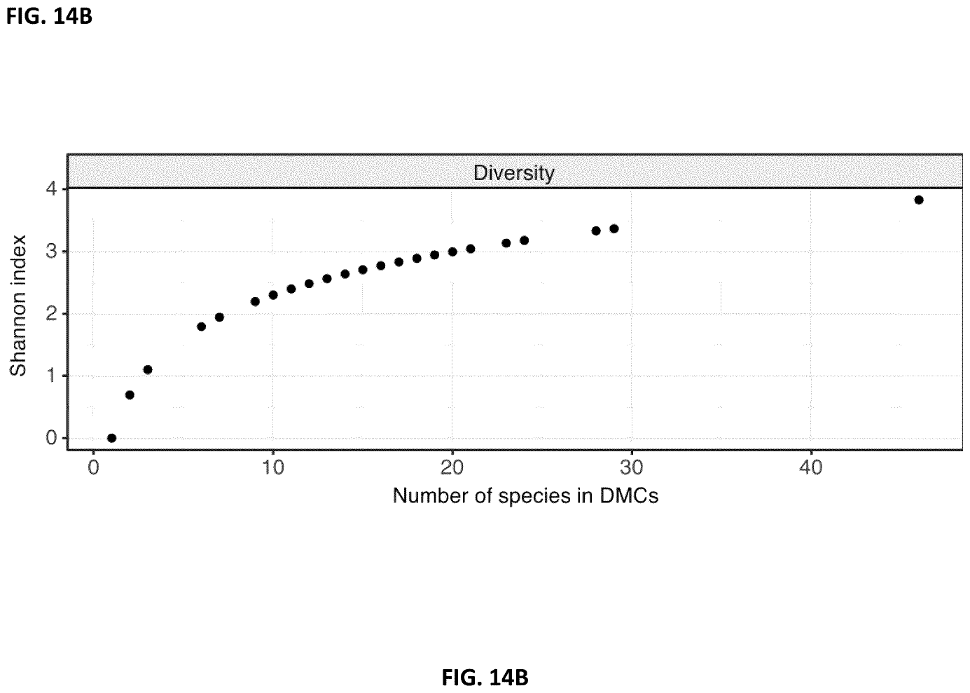

This process is outlined for a community comprised of three strains in FIG. 14A.

Creating the Klebsiella pneumoniae MH258 strain used in experiments: The K. pneumoniae-MH258 isolate was previously described elsewhere³¹. For better in vitro and in vivo selection of this strain, K. pneumnoniae-MH258 was transformed by electroporation with pmCherry-sfGFP (86441; ADDGENE®).

Experimental workflow for Kp clearance assay: The 46 bacterial strains described in Supplementary Table 1B were individually inoculated from a frozen stock into 900 μL of BHI supplemented with cysteine 0.1% (BHIS) previously reduced. Strains were incubated at 37° C. in static conditions for 48 hours (h) in anaerobiosis to ensure that the most fastidious strains reached stationary phase. K. pneumioniae-MH258 sGFP was also inoculated in the same conditions, but only 24 h after commensal isolates inoculation due to the fast growth capacity of this species and was incubated for 24 h. All strain densities were assessed by taking 100 μL of each culture and measuring OD₆₀₀ in an imager (Cytation 5, BIOTEK®). To build all DMCs, isolates were inoculated in 900 μL of BHIS previously reduced in different combinations with an initial OD₆₀₀ of 0.001, so that the densest community reached a maximum total initial OD₆₀₀ of approximately 0.05. K. pneumoniae was added at the same initial OD₆₀₀ of 0.001 to all DMCs. Cultures were incubated at 37° C. in static conditions and anaerobiosis for 5 days. To assess K. pneumoniae abundance, 10 μL of each culture were collected daily and homogenized in 90 μL of PBS and serially diluted. Diluted samples were plated in BHIS with kanamycin (50 μg/mL). Plates were incubated at 37° C. overnight in aerobiosis. GFP expressing K. pneumoniae-MH258 colony forming units (CFUs) were enumerated. In parallel, 100 μL of each culture was also collected to recover the cell phase and the supernatants at 72 h, 96 h, and 120 h. These samples were stored at −80° C. to be later processed for shotgun metagenomics and metabolomics.

Training and validation of Random Forest (RF) model: The inventors used a RandomForestRegressor, available with scikit-learn python package³³. Tree Depth was set to 12 levels per tree, the number of trees was set to 100, and the maximum number of features was set to “sqrt” (square-root of the number of strains total). Out-of-bag error was measured by a combination of R\hat{ }2 (where numbers less than 1 indicated more error) and Mean Squared Error (where larger numbers indicated more error). To train and validate the model, the inventors randomly split the dataset into 90% training and 10% true-out-of-sample 100 times. The input data was a vector of 46 1's and 0's as shown in the matrix displayed in FIG. 7E corresponding to the pattern of presence-absence for each DMC. In each iteration, the RandomForestRegressor was fit to the training set via 6-fold cross validation. Cross-validation accuracy was measured through Pearson Correlation. The true out of sample set was then predicted, and prediction accuracy was measured by computing Mean Squared Error and Pearson Correlation of the predicted versus measured K. pneumoniae abundances after 120 hours of co-culture with the DMC. Feature Importance Scores for all features were observed and stored. This process was repeated 100 times, and prediction accuracies and feature significance scores were averaged. An additional RandomForestRegressor model was then trained on the entirety of the dataset with 6-fold cross-validation. Cross-validation accuracy was measured by calculating Mean Squared Error, Pearson Correlation, and R\hat{ }2. Averaged prediction accuracies and feature significance scores were used to estimate prediction error.

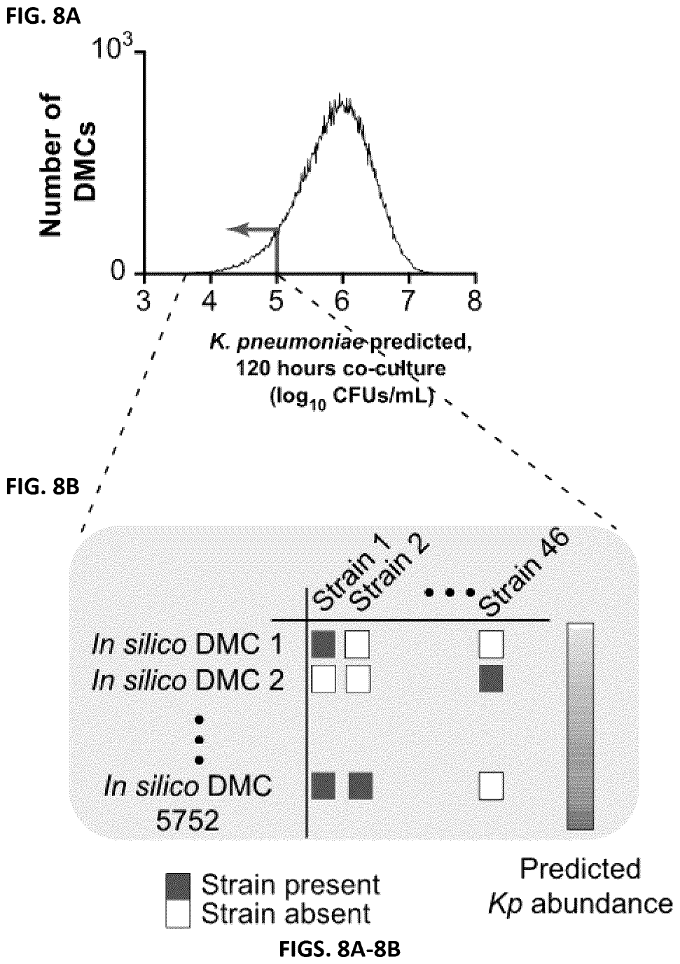

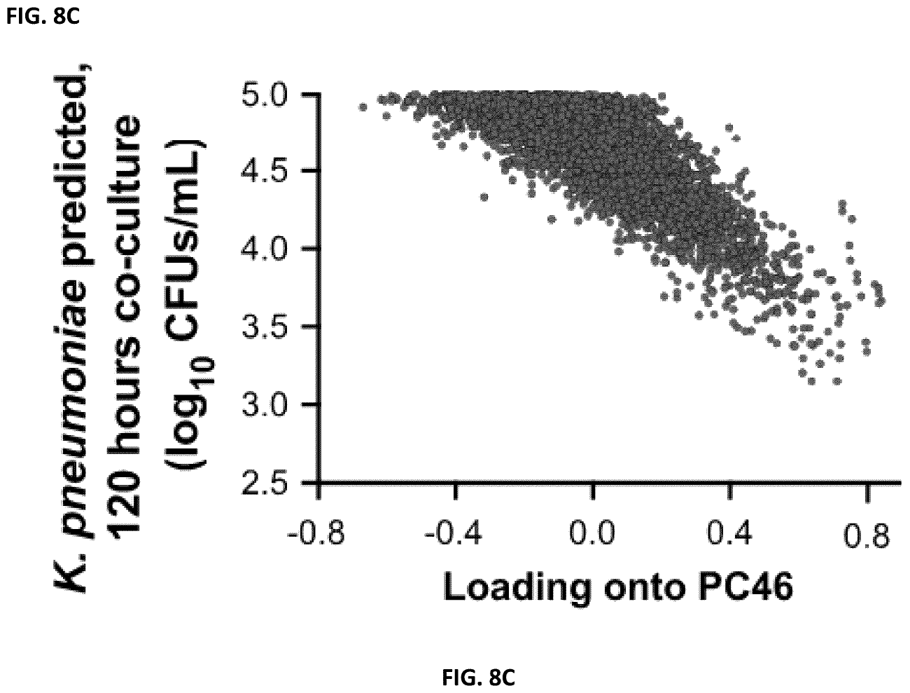

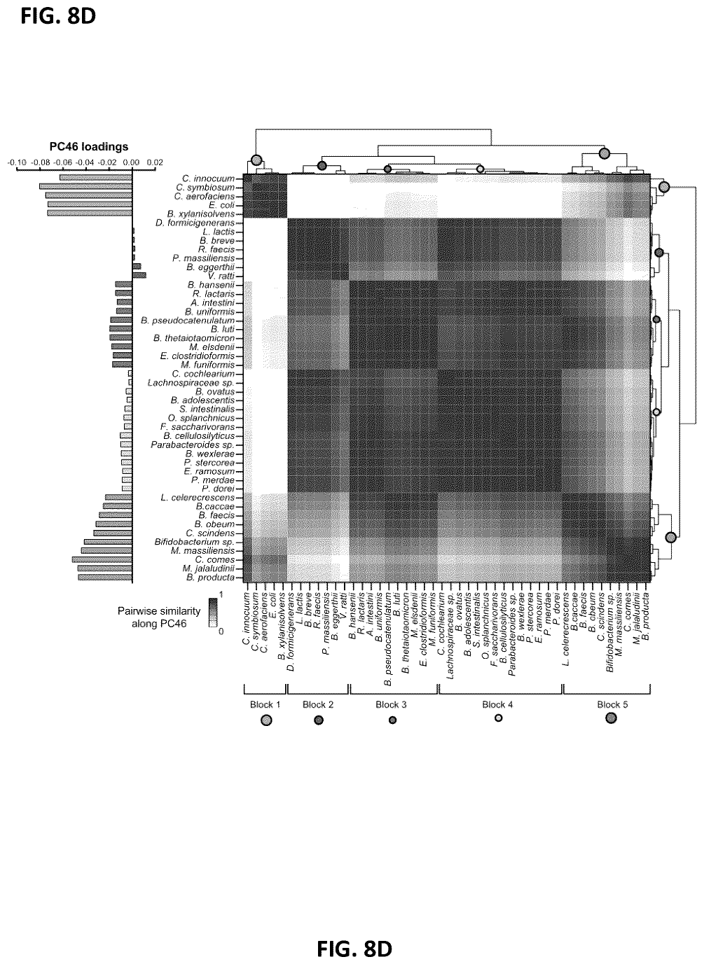

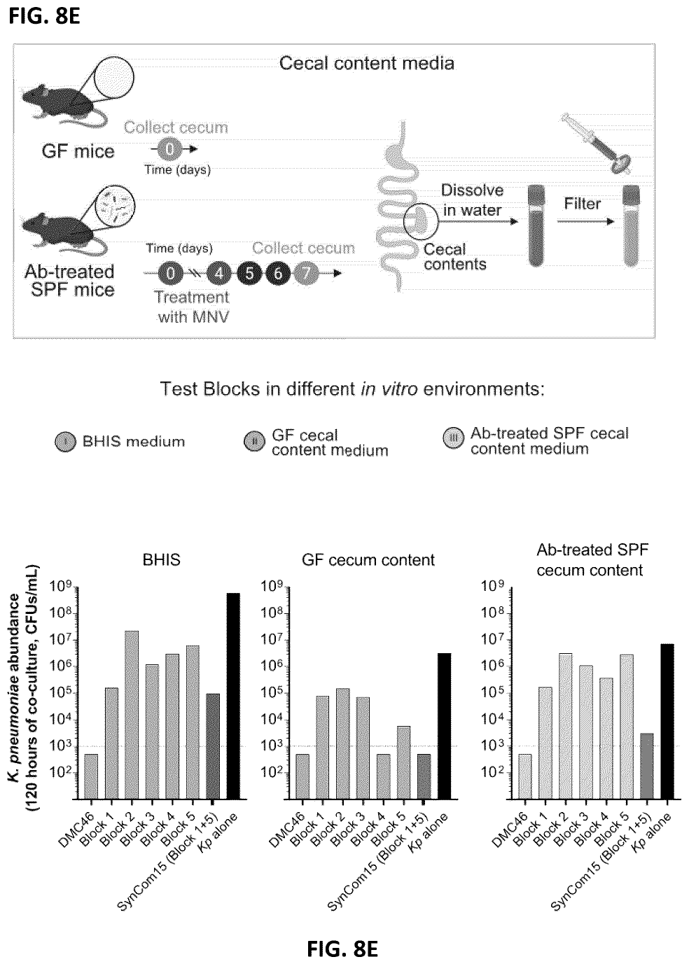

Statistical analysis of matrix in FIG. 8B: The matrix in FIG. 8B was subject to PCA resulting in 46 principal components of data-variance (eigenvectors). The inventors found that the first principal component (PC1) was significantly associated with community complexity (Supplementary Table 6C). To isolate the effect of K. pneumoniae clearance from community size, the inventors first performed a series of steps to “regress out” the effect of community size. First, let xᵢ be the community size of DMC i. Let yᵢ be the predicted K. pneumoniae clearance from the RF model for DMC i. A linear model is then created regressing community size against K. pneumoniae clearance taking the form:

$${y}_{i} = {β}_{1} {x}_{i} + {β}_{0} + ε \qquad ( 5 )$$

$$= {β}_{1} {x}_{i} + {β}_{0} \qquad ( 6 )$$

where [symbol] is the K. pneumoniae clearance of DMC i as a function of its size. The residuals of [symbol] this linear model are given by

$${ε}_{i} = {y}_{i} - {\hat{y}}_{i} \qquad ( 7 )$$

where εᵢ is the degree of K. pneumoniae clearance of DMC i after removing linearly modeled information related to the size of the DMC. All principal components were regressed against rᵢ and principal component 46 (PC46) was found to be the most significantly associated with predicted, residualized K. pneumoniae clearance (Supplementary Table 6D).

Defining the matrix in FIG. 8D: Let u ∈ [symbol] be the vector of column projections [symbol] of each strain on PC46 of the matrix defined in FIG. 2B.

Let s be the scalar value denoting the maximum value of u

$${A}^{46 \times 46} = ( {a}_{ij} ) \qquad ( 8 )$$

$${a}_{ij} = {d}_{ij}^{2} = {‖ {u}_{i} - {u}_{j} ‖}^{2} \qquad ( 9 )$$

Where ∥⋅∥ denotes the Euclidian norm on

[symbol]

$${A}_{i , j} = [ \begin{matrix}0 & … & {d}_{46 , 1}^{2} \\ ⋮ & ⋱ & ⋮ \\ {d}_{1 , 46}^{2} & … & 0\end{matrix} ] \qquad ( 10 )$$

$${S}_{i , j} = s - {A}_{i , j} = [ \begin{matrix}s & … & s - {d}_{46 , 1}^{2} \\ ⋮ & ⋱ & ⋮ \\ s - {d}_{1 , 46}^{2} & … & s\end{matrix} ] \qquad ( 11 )$$

The resulting symmetric similarity matrix, S_{i,j}, with rows and columns indicating each strain and each element representing the similarity between strain i and strain j describes how strains are related to one another based on their projections along PC46. Hierarchical clustering on the resulting similarity matrix was then performed to identify groups of strains. Strains that are more similar are often found in communities that suppress K. pneumoniae and those that are more distant are rarely found in communities that suppress K. pneumoniae.

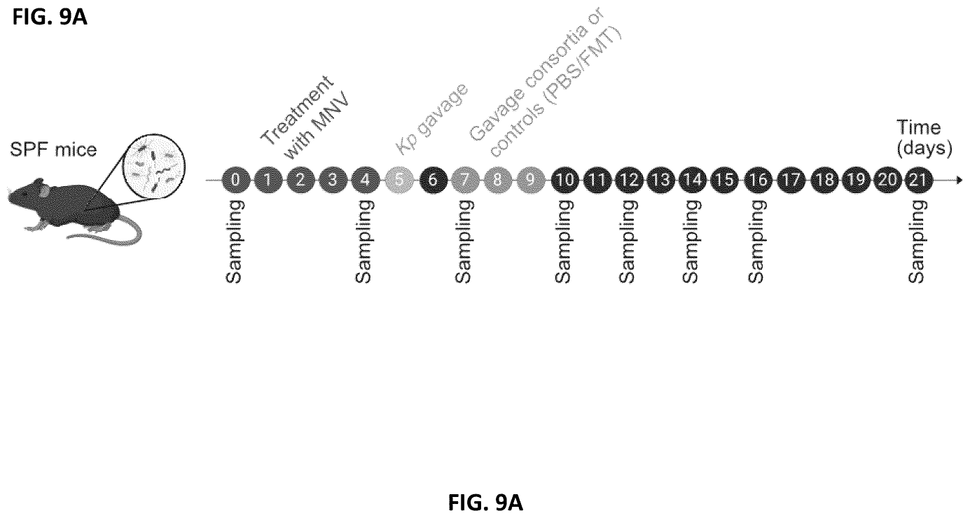

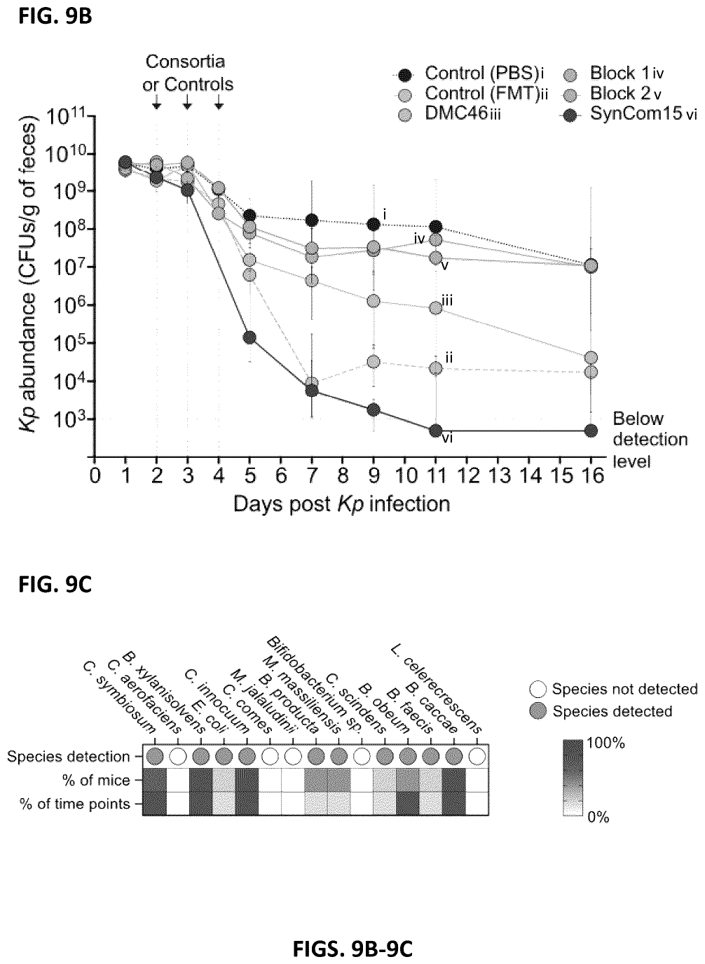

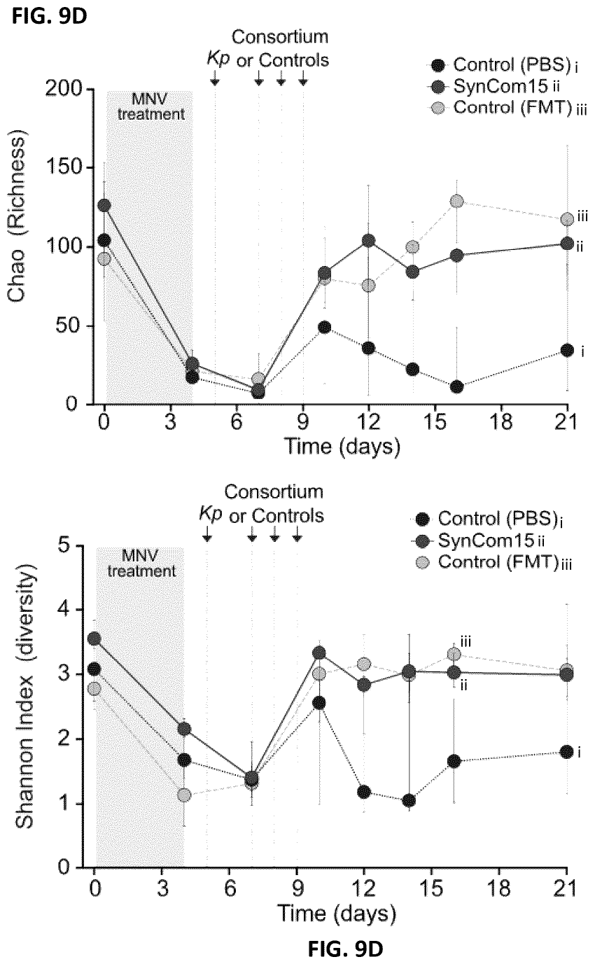

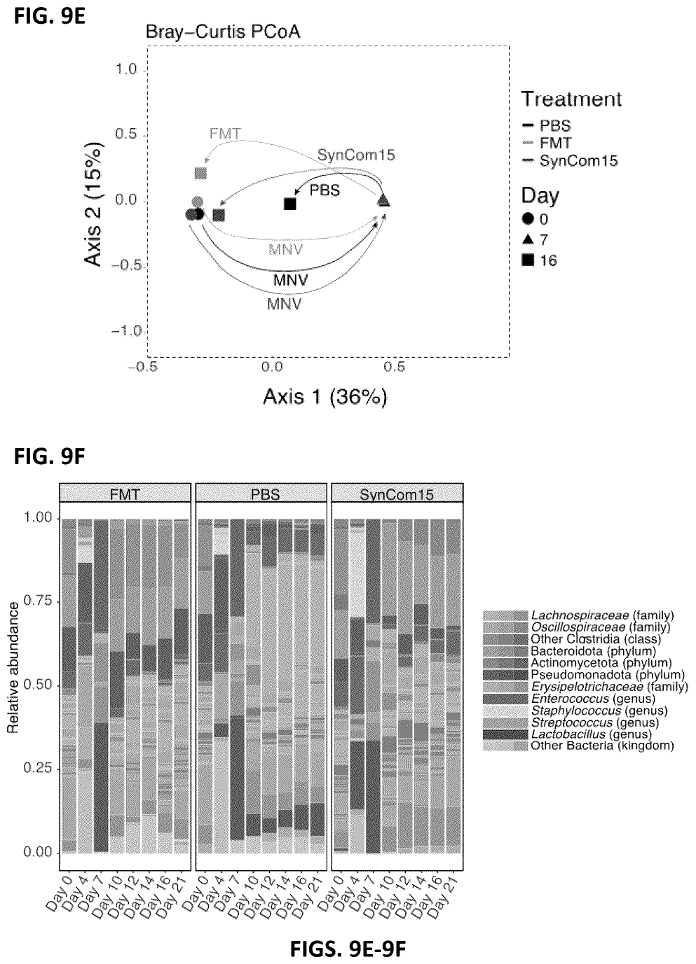

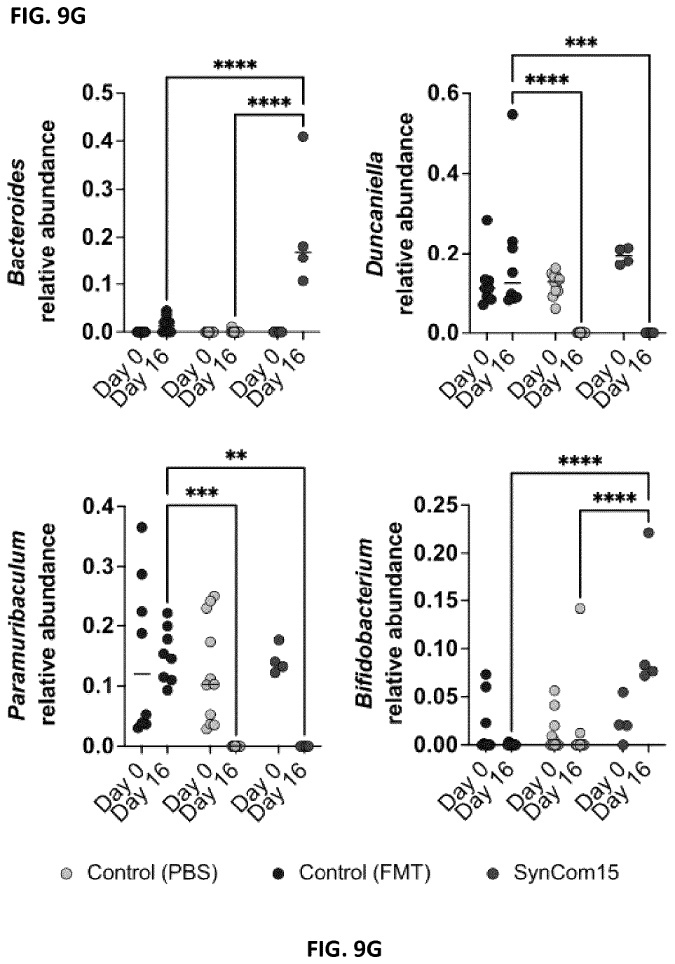

Characterization of mice used for all experiments spanning FIG. 8 and FIG. 9: All mouse experiments were performed in accordance with and approved by the Institutional Animal Care and Use Committee of the University of Chicago under protocol 72599. Male specific-pathogen-free C57BL/6J mice, aged 8 weeks to 10 weeks, from Jackson Laboratories were used for all experiments. Mice were kept within a facility that maintained a 12 hour light and 12 hour dark cycle and controlled humidity (30-70%) and temperature (68-79° F.). Mice were housed in sterile, autoclaved cages with irradiated feed (5K67, LABDIETS®) and acidified, autoclaved water upon arriving at the on-site mouse facility. Mouse handling and cage changes were performed by investigators wearing sterile gowns, masks and gloves in a sterile biosafety hood. Mice were cohoused with their original shipment group until starting the experiment.

For germ-free (GF) studies, 8-10-week-old wild-type male C57BL/6J mice were used for all studies. Mice were initially obtained from The Jackson Laboratory and subsequently bred and raised in a GF isolator. After removal from the GF isolator, mice were handled in a sterile manner and individually housed in sealed negative pressure bio-containment unit isolators. Throughout breeding, mice were housed within the University of Chicago Gnotobiotic Research Animal Facility (GRAF) and maintained at a 12 hour light and 12 hour dark cycle and controlled humidity (30-70%) and temperature (68-79° F.). Gnotobiotic mice were fed an ad libitum diet of autoclaved Teklad Global 18% Protein Rodent Diet (Sterilizable) (2018S/2018SC).

Creating GF and antibiotic (Ab)-SPF cecal extract media: To create GF cecal extract media, 8-10-week-old wild-type male C57BL/6J GF mice were euthanized and cecal contents were collected, weighted, and homogenized in 10 mL of sterile distilled water per gram of content. Cecal suspension was centrifuged, and supernatants were filtered through a 0.22 μm filter. GF cecal extract media was stored at −80° C.

To create ab-SPF cecal extract media C57BL/6J SPF male mice at 8-10 weeks of age were singly housed and placed under an antibiotic regime (0.25 g of MNV—metronidazole, neomycin, and vancomycin) in the drinking water (day 0). Four days later, antibiotic treatment was halted and mice were placed on normal acidified water (day 4). Cages and food were also changed. On day 7 mice were euthanized and cecal contents were collected, weighted, and homogenized in 10 ml, of sterile distilled water per gram of content. Cecal suspension was centrifuged, and supernatants were filtered through a 0.22 μm filter. Ab-SPF cecal extract media was stored at −80° C.

K. pneumoniae clearance in cecal extract media: DMCs capacity to inhibit Kp was tested by individually inoculating the 46 isolates from a frozen stock into 900 μL of BHIS previously reduced. Strains were incubated at 37° C. in static conditions for 48 h in anaerobiosis. Kp was also inoculated in the same conditions, but only 24 h after commensal isolates inoculation, and was incubated for 24 h. The density of each and all isolates was assessed by taking 100 μL of each culture and measuring OD₆₀₀ in an imager (Cytation 5, BIOTEK®). To build all defined bacterial consortia, isolates were inoculated in 900 μL of either GF or Ab-SPF cecal extract media previously reduced in different combinations with an initial OD₆₀₀ of 0.001, so that the densest community reached a maximum total initial OD₆₀₀ of approximately 0.05. To all defined communities, K. pneumoniae was added at the same initial OD₆₀₀ of 0.001. Cultures were incubated at 37° C. in static conditions and anaerobiosis for 5 days. To assess for K. pneumoniae levels 10 μL of each culture were collected daily and homogenized in 90 μL of PBS and serially diluted. Diluted samples were plated in BHIS with kanamycin (50 μg/mL). Plates were incubated at 37° C. overnight in aerobiosis. GFP expressing K. pneumoniae CFUs were enumerated. In parallel, 100 μL of each culture was also collected to recover the cell phase and the supernatants at 72 h, 96 h, and 120 h. These samples were stored at −80° C. to be later processed for shotgun metagenomics and metabolomics.

Preparation of mice stool samples for fecal microbiota transplant (FMT): Fecal samples from 15-20 mice SPF mice from different cages (to increase sample diversity) were collected in a 50 mL tube. Samples were transferred immediately to the anaerobic chamber (anaerobic exposure was kept under 30 min). Samples were dissolved in 1 mL of PBS 20% glycerol 0.1% cysteine (previously filtered and reduced) per fecal pellet (1 mL per ˜20 mg of fecal sample) using a mechanical pestle and vortexing. Samples were aliquoted in cryovials and stored −80° C. until use.

SPF mouse model ofK. pneumoniae infection: C57BL/6J male mice of 8-10 weeks of age were singly housed and placed under an antibiotic regime (0.25 g of MNV—metronidazole, neomycin, and vancomycin) in the drinking water (day 0). Four days later, antibiotic treatment was halted and mice were placed on normal acidified water (day 4). Cages and food were also changed. On day 5 all mice were gavaged with 100 μL of PBS containing 500 CFUs of K. pneumoniae, prepared as previously explained. On days 7, 8, and 9 mice were gavaged with 100 μL of either selected defined bacterial consortia, a fecal microbiota transplant from naïve healthy mice, or PBS. Fecal samples were collected on days 0, 4, 7, 10, 12, 14, 16, and 21 (final day of the experiment) for 16S rRNA sequencing and on day 10 and 12 for metabolomics. These were immediately place on dry ice after collection and later stored at −80° C. To assess for K. pneumoniae levels, fecal samples were collected on days 7, 10, 12, 14, 16, and 21. Fecal samples were homogenized in 1 mL of PBS and serially diluted. Undiluted and diluted samples were plated in BHIS and kanamycin (50 μg/mL).

Determining engraftment of SynComi5 strains in SPF mice: To determine SynCom15 strain engraftment, 16S rRNA sequences from all 15 strains were blasted against 16S rRNA sequences derived from fecal samples of antibiotic-treated SPF mice gavaged with SynCom15 consortium. Fecal-derived sequences were assigned to a SynCom15 strain if their 16S rRNA percentage sequence identity was 100% with a minimum of a 95% coverage.

Determining structure of microbiota in infected SPF mice given saline, FMT, or SynCom15: DNA was extracted using the QIAamp PowerFecal Pro DNA kit (QIAGEN®). Before extraction, samples were subjected to mechanical disruption using a bead beating method. Briefly, samples were suspended in a bead tube (QIAGEN®) along with lysis buffer and loaded on a bead mill homogenizer (FISHERBRAND®). Samples were then centrifuged, and supernatant was resuspended in a reagent that effectively removed inhibitors. DNA was then purified routinely using a spin column filter membrane and quantified using fluorometric quantification (Qubit, INVITROGEN®).

16S sequencing was performed for murine studies, where the V4-V5 region within the 16S rRNA gene was amplified using universal bacterial primers-563F (5′-nnnnnnnn-NNNNNNNNNNNN-AYTGGGYDTAAA-GNG-3′ (SEQ ID NO: 15)) and 926R (5′-nnnnnnnn-NNNNNNNNNNNN-CCGTCAATTYHT-TTRAGT-3′ (SEQ ID NO: 16)), where ‘N’ represents the barcodes and ‘n’ are additional nucleotides added to offset primer sequencing. Then, amplicons of approximately 412 bp were purified using a spin column-based method (Minelute, QIAGEN®), quantified and pooled at equimolar concentrations. Illumina sequencing-compatible Unique Dual Index adapters were ligated onto the pools using the QIAseq 1-step amplicon library kit (QIAGEN®). Library quality control was performed using Qubit and TapeStation and sequenced on Illumina MiSeq platform to generate 2×250 bp reads.

Raw V4-V5 16S rRNA gene sequencing data was demultiplexed and processed through the dada2 pipeline (v1.18.0) into amplicon sequence variants (ASVs) with minor modifications in R (v4.0.3)⁶⁶. Specifically, reads were first trimmed at 190 bp for both forward and reverse reads to remove low-quality nucleotides. Chimeras were detected and removed using the default consensus method in the dada2 pipeline. Then, ASVs with a length between 320 bp and 365 bp were kept and deemed as high-quality ASVs. Taxonomy of the resultant ASVs was assigned to the genus level using the RDP Classifier (v2.13) with a minimum bootstrap confidence score of 80⁶7.

Comparison of SynCom15 with microbiotas of healthy human donors: To investigate the presence of SynCom15 strains in samples from healthy human donors, SynCom15 strains taxonomic names were searched in the 22 fecal samples obtained from the DFI 22 human donors. For SynCom15 strain unclassified to species level Bifidobacterium sp., the most closely related species annotated by GTDB with an 98,21% ANI (Bifidobacierium pseudocatenulatum) was used^{62,68}.

Metabolic profiling of designed communities: For metabolite extraction from liquid cultures, samples were incubated at −80° C. between 1 h and 12 h. Four volumes of methanol spiked with internal standards were added to each culture supernatant. Samples were then centrifuged at −10° C. and 20,000×g for 15 min followed by the transfer of 100 μL of supernatant to pre-labelled mass spectrometer autosampler vials (09-1200, MICROLITER®).

For metabolite extraction from fecal samples, extraction solvent (80% methanol spiked with internal standards and stored at −80° C.) was added at a ratio of 100 mg of material/mL of extraction solvent in beadruptor tubes (15-340-154, FISHERBRAND®). Samples were homogenized at 4° C. on a Bead Mill 24 Homogenizer (15-340-163, FISHER®), set at 1.6 m/s with 6 thirty-second cycles, 5 seconds off per cycle. Samples were then centrifuged at −10° C., 20,000×g for 15 min and the supernatant was used for subsequent metabolomic analysis.

Short chain fatty acids were derivatized as described by Haak et al. with the following modifications⁶⁹. The metabolite extract (100 μL) was added to 100 μL of 100 mM borate buffer (pH 10) (28341, THERMO FISHER®), 400 μL of 100 mM pentafluorobenzyl bromide (90257, MILLIPORE SIGMA®) in Acetonitrile (A955-4, FISHER®), and 400 μL of n-hexane (160780010, ACROS ORGANICS®) in a capped mass spec autosampler vial (09-1200, MICROLITER®). Samples were heated in a thermomixer C (EPPENDORF®) to 65° C. for 1 hour while shaking at 1300 rpm. After cooling to room temperature (RT), samples were centrifuged at 4° C., 2000×g for 5 min, allowing phase separation. The hexanes phase (100 μL) (top layer) was transferred to an autosampler vial containing a glass insert and the vial was sealed. Another 100 μL of the hexanes phase was diluted with 900 μL of n-hexane in an autosampler vial. Concentrated and dilute samples were analyzed using a GC-MS (AGILENT® 7890A GC system, AGILENT® 5975C MS detector) operating in negative chemical ionization mode, using a HP-5MSUI column (30 m×0.25 mm, 0.25 m; 19091S-433UI, AGILENT TECHNOLOGIES®), methane as the reagent gas (99.999% pure) and 1 L split injection (1:10 split ratio). Oven ramp parameters: 1 min hold at 60° C., 25° C. per min up to 300° C. with a 2.5 min hold at 300° C. Inlet temperature was 280° C. and transfer line was 310° C. A 10-point calibration curve was prepared with acetate (100 mM), propionate (25 mM), butyrate (12.5 mM), and succinate (50 mM), with 9 subsequent 2× serial dilutions.

Metabolites were also analyzed using gas chromatography mass spectrometry (GC-MS) with electron impact ionization. The metabolite extract (100 L) mass spec autosampler vials (09-1200, MICROLITER®) and dried down completely under nitrogen stream at 30 L/min (top) 1 L/min (bottom) at 30° C. (SPE Dry 96 Dual, 3579M, BIOTAGE®). To dried samples, 50 μL of freshly prepared 20 mg/mL methoxyamine (226904, SIGMA®) in pyridine (270970, SIGMA®) was added and incubated in a thermomixer C (EPPENDORF®) for 90 min at 30° C. and 1400 rpm. After samples are cooled to room temperature, 80 L of derivatizing reagent (BSTFA+1% TMCS; B-023, SIGMA®) and 70 L of ethyl acetate (439169, SIGMA®) were added and samples were incubated in a thermomixer at 70° C. for 1 hour and 1400 rpm. Samples were cooled to RT and 400 L of Ethyl Acetate was added to dilute samples. Turbid samples were transferred to microcentrifuge tubes and centrifuged at 4° C., 20,000×g for 15 min. Supernatants were then added to mass spec vials for GCMS analysis. Samples were analyzed using a GC-MS (AGILENT® 7890A GC system, AGILENT® 5975C MS detector) operating in electron impact ionization mode, using a HP-5MSUI column (30 m×0.25 mm, 0.25 μm; 19091S-433UI, AGILENT TECHNOLOGIES®) and 1 μL injection. Oven ramp parameters: 1 min hold at 60° C., 16° C. per min up to 300° C. with a 7 min hold at 300° C. Inlet temperature was 280° C. and transfer line was 300° C.

Data analysis was performed using MassHunter Quantitative Analysis software (version B.10, AGILENT TECHNOLOGIES®) and confirmed by comparison to authentic standards. Normalized peak areas were calculated by dividing raw peak areas of targeted analytes by averaged raw peak areas of internal standards.

Training an RF model on metabolic content: First, Z-scores of all metabolites were centered and normalized. This was done by subtracting the mean Z score from the observed Z score and dividing it by the standard deviation of Z scores. This normalization ensured that for each metabolite, the distribution across all communities was zero and its standard deviation was one. With respect to output, a pseudocount of 10 was added to all K. pneumoniae values to enable prediction of the decadic logarithm (log₁₀) of K. pneumoniae abundance.

After standardization, 50% of the data was used for training and the remaining 50% for validation. A RF model was built with 10,000 trees with mean squared error minimization as the strategy for training. The number of features chosen by each tree was set to 10, based on the square root of the total number of metabolites available to profile. This feature selection was optimized by testing model performance with a feature range between 2 and 50. The model displayed stable performance when the number of features per tree was between 7 and 20. Below 7, the model performance degraded due to insufficient information on relationships between metabolite features; above 20, the RF trees became too similar thereby impacting overall model effectiveness by skewing the final decision output by the model. Once trained, the RF model was tested on the training, test, and out-of-sample tests.

### Example 4—Statistical Design of a Synthetic Microbiome that Cleared a Multi-Drug Resistant Gut Pathogen

Abstract: Microbiomes perform critical functions across many environments on Earth¹⁻³. However, elucidating principles of their design is immensely challenging⁴⁻⁷. Using a diverse bank of human gut commensal strains and clearance of multi-drug resistant Klebsiella pneumoniae as a target, the inventors engineered a functional synthetic microbiome using a process that was agnostic to mechanism of action, bacterial interactions, or compositions of natural microbiomes. The strategy of the inventors was a modified ‘Design-Build-Test-Learn’ approach (‘DBTL+’) coupled with statistical inference that learned design principles by considering only the strain presence-absence of designed communities. In just a single round of DBTL+, the inventors converged on a generative model of K. pneumoniae suppression. Statistical inference performed on the model identified 15 strains that were key for community function. Combining these strains into a community (‘SynCom15’) suppressed K. pneumoniae across unrelated in vitro environments and matched the clearance ability of a whole stool transplant in a pre-clinically relevant mouse model of infection. Considering metabolic profiles of communities instead of strain presence-absence yielded a poor generative model, demonstrating the advantage of using strain presence-absence for deriving principles of community design. The inventor's work introduced the concept of “statistical design” for engineering synthetic microbiomes, opening the possibility of synthetic ecology more broadly.

Main: Using a diverse collection of human gut commensal strains, the inventors sought to engineer a bacterial microbiome that could clear multi-drug resistant (MDR) K. pneumoniae—a pathogen classified in the “Priority 1: Critical” category of antibiotic resistant organisms by the World Health Organization²⁴. Towards this goal, the inventors implemented a “Design-Build-Test-Learn” (DBTL) approach that was different from a traditional DBTL framework in two ways. First, the initial round of community design was subject to a constraint: maximizing genomic diversity of constituent bacterial strains. The rationale in implementing this constraint was to minimize potential functional redundancy in constructed communities. Second, a model of community function was statistically learned by considering only the pattern of strain presence-absence for designed communities, thereby remaining agnostic to many parameters that could influence community structure and function. This approach was termed “DBTL+.” Implementing just a single round of DBTL+ wherein 96 “designed microbial communities” (DMCs) were built and tested resulted in an accurate generative statistical model of community design for suppressing K. pneumoniae in an in vitro setting. Statistical inference performed on this model identified a set of 15 key strains that when combined into a community (‘SynCom15’) (i) sustainably suppressed K. pneumoniae across various diverse in vitro environments; (ii) matched the clearance ability of a fecal microbial transplant (FMT) in a pre-clinically relevant mouse model of infection; (iii) was a safe intervention in vivo; (iv) could not be obviously deconstructed into a functional subset of strains; and (v) did not resemble the composition of natural human gut microbiotas. The inventors found that considering the metabolic capacity of DMCs including fatty acid and nutrient metabolism—appreciated mechanisms of K. pneumoniae suppression—instead of strain presence-absence resulted in a poor generative model, highlighting the advantage of describing DMCs by their strain content for deriving generative models of community design²¹⁻²⁷. The inventors' work described a potentially therapeutic, sparse synthetic microbiome made of human gut commensal bacteria for treatment of MDR K. pneumoniae infections and, more generally, introduced the concept of “statistical design” for microbial ecosystems.

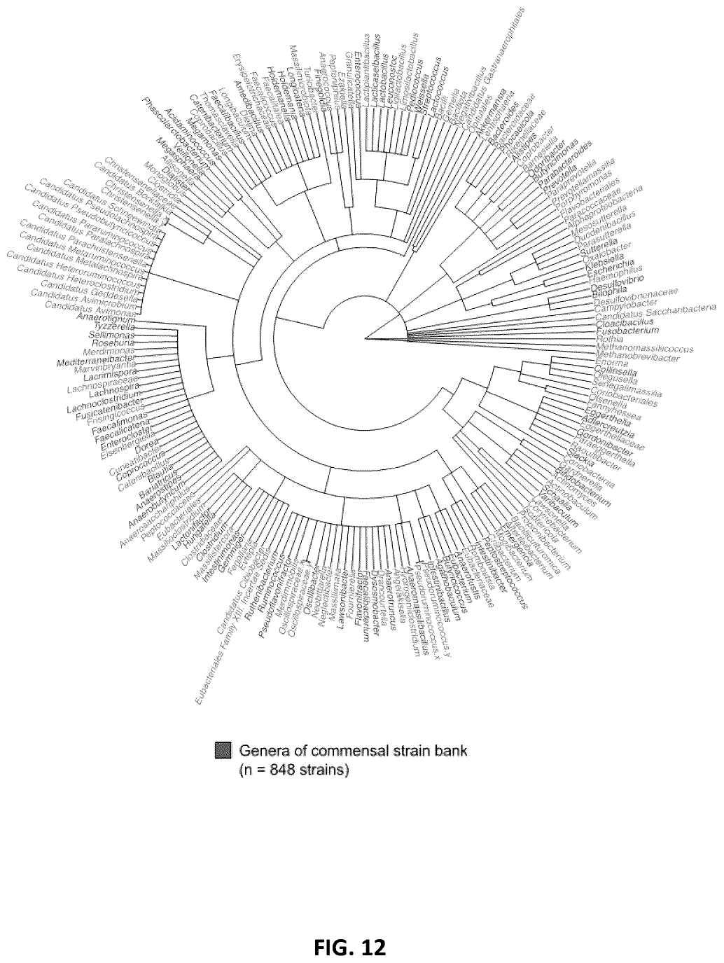

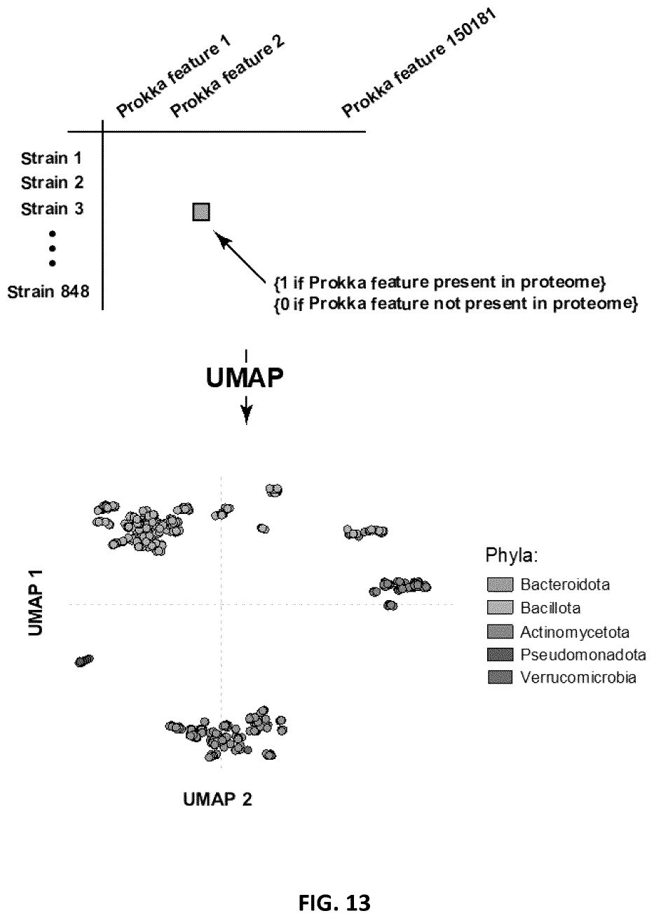

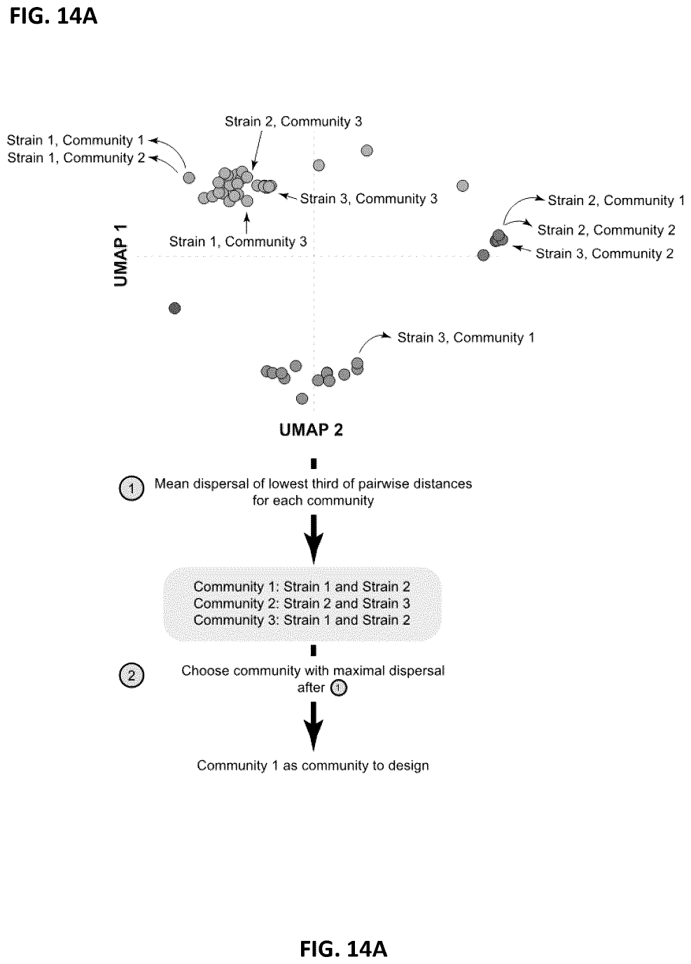

A generative model of community design for suppressing K. pneumoniae: To begin the DBTL+ approach, the inventors first isolated and whole-genome sequenced 848 gut commensal strains from fecal samples of healthy donors (FIGS. 7A-7B; Supplementary Table 1; Example 3). The strain bank was enriched for the phyla Bacteroidota, Bacillota, Actinomycetota, Pseudomonadota, and Verrucomicrobia, and contained a richness of diversity at the genus and species levels reflecting the diversity of donor microbiomes (FIG. 7B, FIGS. 12-13; Supplementary Information; Example 3). The possible combinatorial space of DMCs the inventors could synthesize was 2⁸⁴⁸/2—an insurmountable number. As such, the inventors reduced the size of the strain bank while maintaining the genomic diversity of the resulting set (FIG. 7C; Supplementary Table 2A). The inventors chose 46 strains as the size for the reduced strain bank because creating a nearly 50-member community was the practical limit that could be achieve without compromising the fitness of bacteria in culture. Despite substantially reducing the size of the strain bank, the possible combinatorial space of communities was still ˜2⁴⁶/2, or 35 trillion, possibilities. Therefore, the inventors implemented a constraint to design the first round of communities. The rationale was to create communities comprised of a diverse set of strains, rather than a set of strains that were closely related to each other, to maximize the potential for functional diversity of a given community. To this end, the inventors used the UMAP space of the 46 strains to design diverse communities (FIG. 8C; Supplementary Table 2B).

To create a diverse community of size N, one option could be to choose the set of N strains that maximize dispersion across the UMAP space. This problem has been encountered in the field of facilities optimization and is known as “the discrete p-dispersion problem”²⁸⁻³⁰ However, this problem is considered “NP complete”—a class of problem in computer science that is formally hard to solve and whose solutions can be verified only in non-polynomial time. Therefore, the inventors created an algorithm to generate diverse communities (Example 3). First, for a community consisting of N out of the 46 strains in the strain bank, 10,000 communities of size N were randomly created. Second, for each of the communities, all pairwise distances (dispersal) between constituent strains were computed based on their respective distances in the UMAP space. Third, for each of the communities, the dispersal values between strains were ordered from largest to smallest. Finally, the community with the maximum mean dispersal of the lowest 30% of all dispersal values between strains was chosen as a DMC to build and test. By choosing the community with the maximal mean dispersal of the most closely related strains (i.e. the lowest 30%), this algorithm enforces the constraint of diversity across the whole community (FIG. 14A). As an example, implementing this algorithm to engineer a five-member DMC would result in a bacterial community spanning different regions of the UMAP space (FIG. 7D).

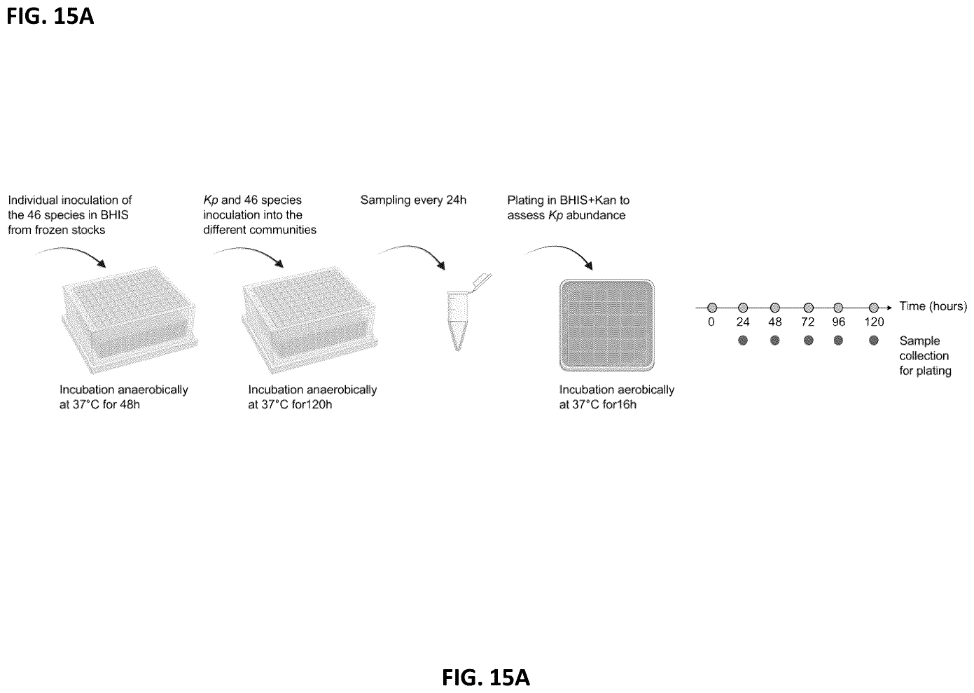

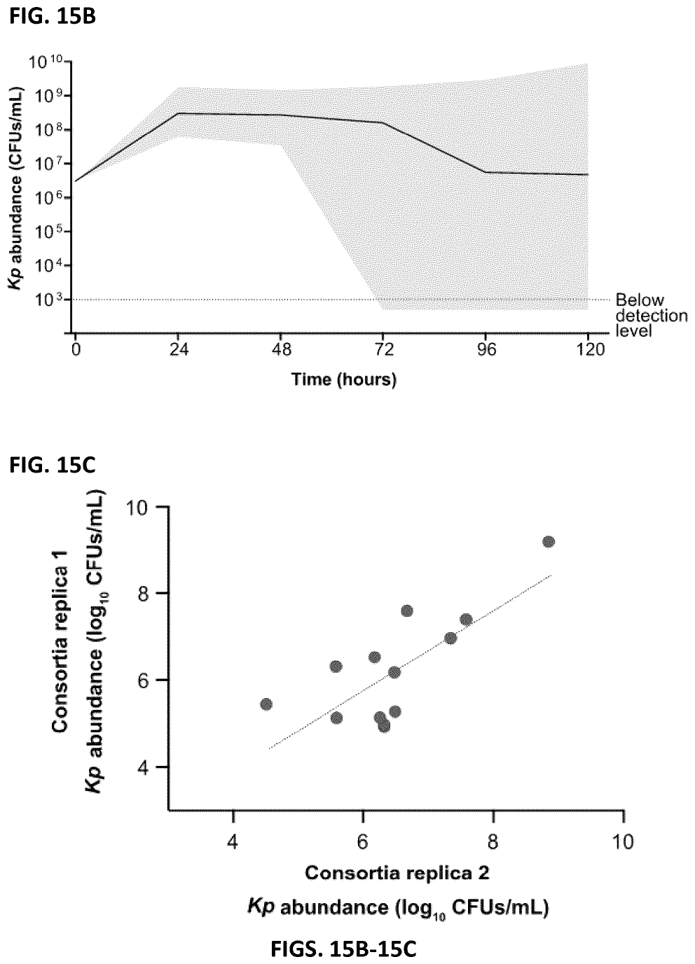

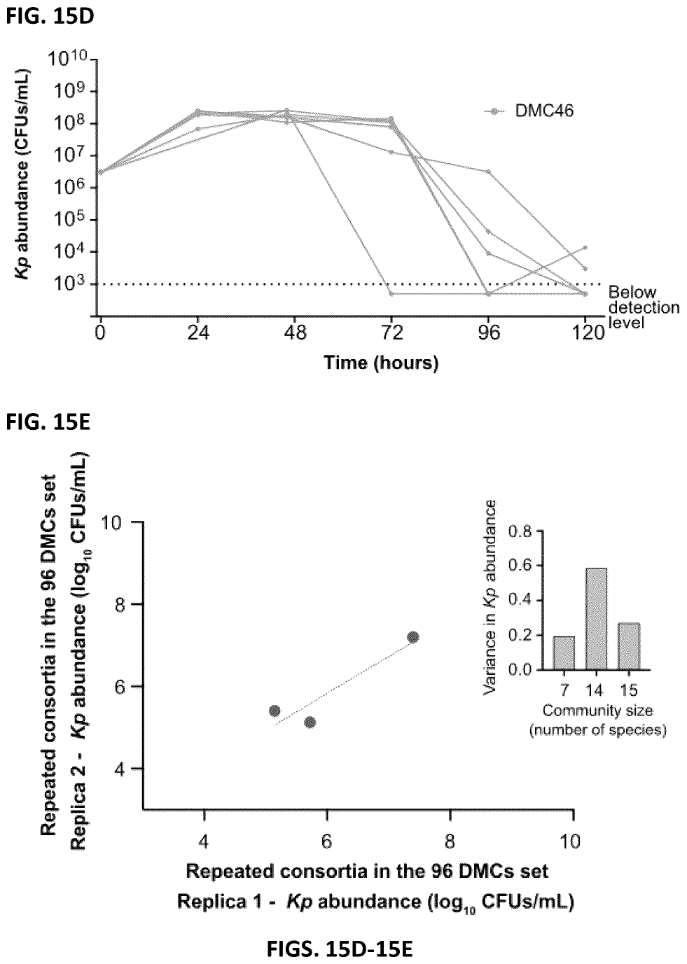

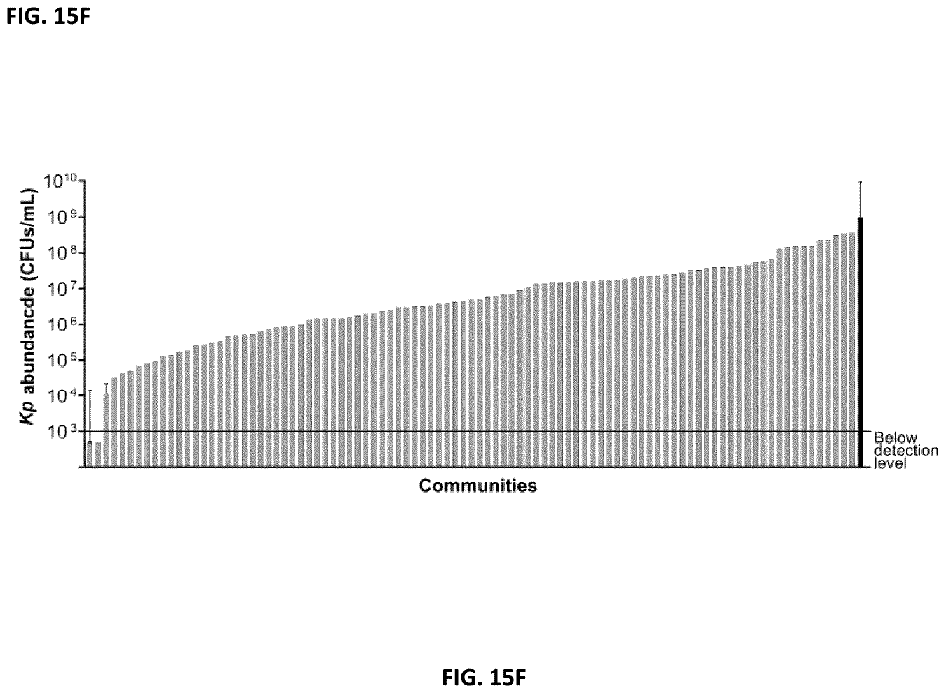

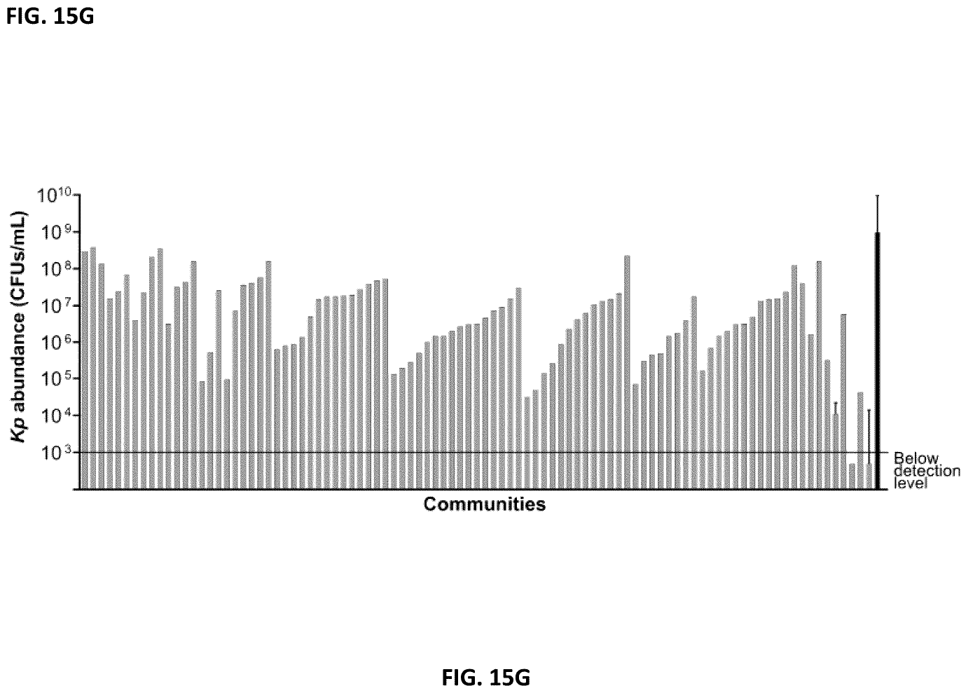

The inventors created 96 DMCs in total—92 diverse DMCs, 3 replicates, and one DMC with all 46 strains (FIG. 7E; Supplementary Table 3A). As the inventors had no prior for constraining the size of the DMCs, the rationale was to span a wide range of membership sizes. The inventors designed the 96 DMCs to span two to 46 strains with the average size being 15 bacterial strains with 5 strains as the standard deviation. As the size of the DMC increased, the Shannon diversity increased as well, illustrating that the strategy of design resulted in metagenomically diverse communities (FIG. 14B). All DMCs were tested for their ability to suppress K. pneumoniae MH258 an MDR strain isolated from a patient sample obtained from Memorial Sloan Kettering Hospital (MH) representative of the epidemic multilocus sequence type (ST) 258 clone harboring the bla_{KPC}-encoded carbapenemase. The inventors chose this strain to use as a target for suppression because it was amongst the most multi-drug resistant strains that have been previously characterized, exhibiting resistance against a diversity of antibiotics³¹. DMCs were co-cultured with a GFP-tagged K. pneumoniae strain MH258 in Brain-Heart-Infused media with cysteine (BHIS) for 120 hours in an anaerobic chamber (Example 3). The abundance of K. pneumoniae during co-culture with DMCs was quantified through time by plating (FIG. 15A; Example 3).

The inventors found that across all DMCs, K. pneumoniae grew for the first 24 hours from an abundance between 10⁶ and 10⁷ to an abundance of 10⁸ on average and remained constant through the next 24 hours (FIG. 15B). After the first 48 hours of co-culture and up to 120 hours, the 96 DMCs reproducibly exhibited a range of capacity to suppress K. pneumoniae spanning no suppression to suppression greater than seven orders of magnitude equivalent to clearing K. pneumoniae given the lower limit of detection for the assay (FIG. 7E, FIGS. 15B-15E; Supplementary Table 3B). The DMC containing all 46 strains (‘DMC46’) suppressed K. pneumoniae the most, while K. pneumoniae alone maintained the highest abundance. Moreover, the inventors found that the suppressive capacity of DMCs was unrelated to the size of community composition or the presence or absence of a stereotyped taxonomic signature (FIG. 7E, FIGS. 15F-15G). This result suggested that it was likely not the presence or absence of a single strain that mediated the suppression of K. pneumoniae, but rather a complex set of microbial interactions.

The inventors trained and validated a Random-Forests (RF) machine-learning algorithm to learn a statistical relationship between DMC design—defined by only the designed pattern of strain presence and absence of DMCs as represented by the matrix shown in FIG. 7E—and the ending K. pneumoniae abundance after co-culture with the community^{32,33}. Thus, no information about which strains engrafted or survived in the culture, strain dynamics during the experiment, ending configuration of the community, information regarding the nature of microbial interactions, or information regarding mechanism of K. pneumoniae suppression was considered when training or validating this model. The RF model was trained on 90% of the data and validated on the remaining 10% 100 times for bootstrap support, resulting in an in-sample validation r² value of 0.98 (Supplementary Table 4A; Example 3).

The inventors then tested the predictive capacity of the RF model for newly constructed DMCs that the model had never seen as a true “out-of-sample” test. The inventors created 60 new DMCs spanning different membership sizes that were not a part of the initial 96 DMCs and were predicted by the trained RF model to span a large dynamic range of K. pneumoniae clearance in the assay (Supplementary Table 5A). Thus the 60 new DMCs defined a true “out-of-sample” set generated by the RF model. The inventors compared the abundance of K. pneumoniae for the 60 new DMCs as predicted by the RF model versus the K. pneumoniae abundance that were experimentally observed after co-culture of each of the 60 new DMCs with K. pneumoniae for 120 hours. The inventors found that the RF model was predictive of the resulting K. pneumoniae abundance to an r² value of 0.6 (p<10⁻³) (FIG. 7F, Supplementary Table 5B).

Collectively, these results showed that the RF model could accurately predict the capacity of a complex microbial community defined by the 46 strains to suppress K. pneumoniae, thereby enabling engineering of new communities with desired suppressive capacity. Thus, in a single round of DBTL where the first round of design was constrained by genomic diversity of strain combinations (DBTL+), the inventors derived a generative model of community design for suppressing K. pneumoniae in BHIS media.

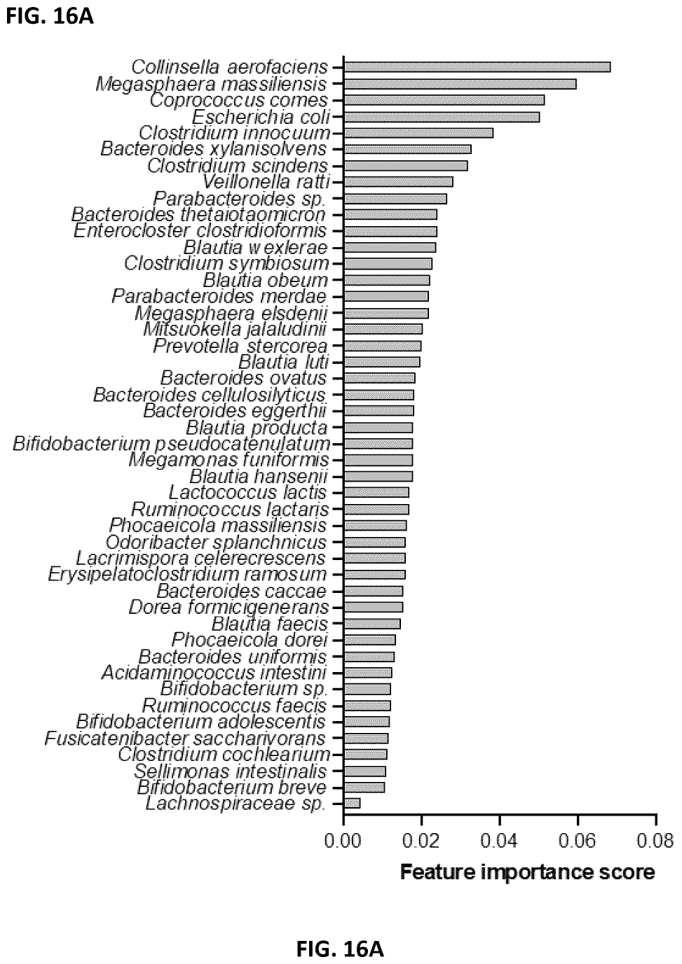

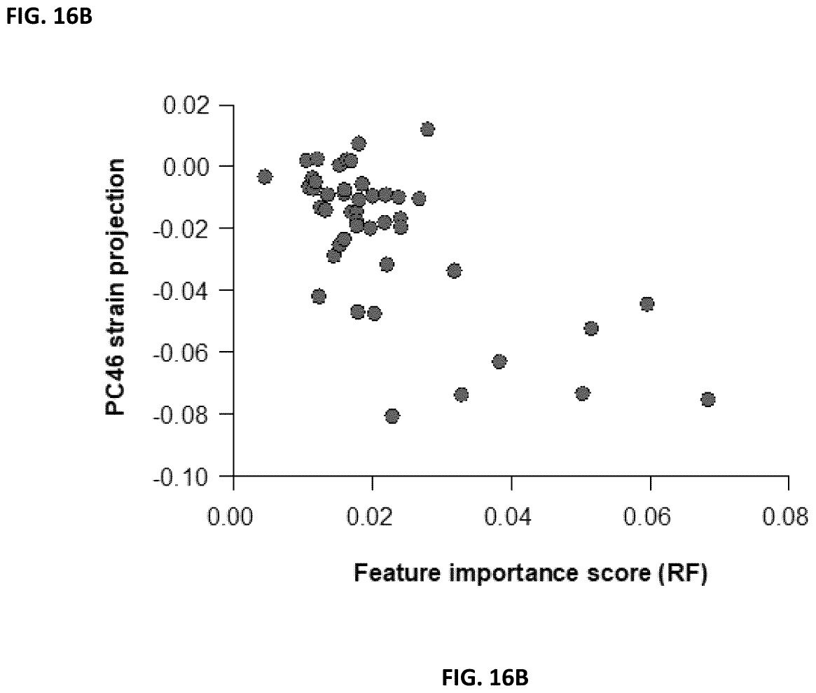

Defining and characterizing SynCom15: The inventors sought to define the critical strains responsible for clearing K. pneumoniae. Current experimental and computational approaches used to define key sets of strains responsible for community function are limited in their abilities to consider higher-order, emergent microbial interactions. In addition, the distribution of feature importance scores generated from the predictive RF model were continuous and therefore unable to delineate groups of important strains (FIG. 16A) (Supplementary Table 4B). Moreover, because RF models are tree-based, they are designed to identify individual features important for prediction, not groups of features. Therefore, the inventors implemented a statistical-inference based strategy initially developed in the field of quantitative finance and then applied it to the study of protein evolution as well as to longitudinal analysis of human microbiomes for identifying groups of collectively interacting parts critical for defining system function. The underlying idea was to first use statistical co-variation between component parts as a proxy for interactions, then to define groups of components that robustly co-vary with each other amongst systems that survive a selective process. Implementing this approach has successfully identified collectives across different scales of complexity: groups of stocks defining economic “sectors,” groups of amino acids defining functional units of proteins (“protein sectors”), and groups of microbes within microbiomes defining covarying units of therapeutic importance (“ecogroups”)^{15,34-39}. The inventors adapted this approach to help identify a collective group of strains critical for suppressing K. pneumoniae.

The inventors scored 100,000 in silico-generated DMCs for their predicted capacity to suppress K. pneumoniae after co-culture using the RF model. The inventors then selected the set of DMCs predicted to suppress K. pneumoniae at least five orders of magnitude (FIG. 8A). The number of DMCs in the resulting set was 5,752. The inventors created an alignment of these DMCs defined by their designed strain presence-absence and labeled each DMC by its K. pneumoniae abundance predicted from the RF model (FIG. 8B, Supplementary Table 6A). Next, the inventors performed Principal Components Analysis (PCA) on the alignment of communities, yielding 46 principal components (PCs) of data-variance. The inventors regressed the contribution of each of the 5,752 DMCs onto each PC against the predicted K. pneumoniae abundance to identify PCs that most associated with K. pneumoniae suppression in a data-driven, unbiased manner. The inventors found that PC46, containing <0.1% data-variance, was the most associated with K. pneumoniae abundance (FIG. 8C, Supplementary Table 6B-D; Example 3).

Similar to the distribution of RF importance scores, the contribution of strains onto PC46 was continuous precluding the ability to define groups of strains to construct communities (FIG. 8D, left panel; Supplementary Table 6E). Interestingly, the contribution of strains onto PC46 did not resemble the distribution of feature importance scores from the RF model, suggesting that PC46 contained information that was different from the RF model (Extended Data FIG. 5B). To use the information in PC46 to define groups of statistically interacting strains, the inventors computed the statistical similarity between all pairs of strains on PC46 (Example 3). The concept behind this measure was that two strains that significantly contribute to PC46 and are close together along PC46 are, on average, co-present in DMCs predicted to suppress K. pneumoniae. Hierarchical clustering of the pairwise similarity between strains illustrated a distinct block structure amongst five separate groups (FIG. 8D, right panel; Supplementary Table 6F). Five strains that contributed the most to defining PC46—Clostridium innocuum, Clostridium symbosium, Colinsella aerofaciens, Escherichia coli, and Bacteroides xylanisolvens-formed a group that was termed “Block 1” (FIG. 8D, right panel). The following ten strains that contributed to PC46—Lacrimispora celerecrescens, Bacteroides caccae, Blautia faecis, Blautia obeum, Clostridium scindens, a Bifidobacterium species, Megasphera massiliensis, Coprococcus comes, Mitsuokella jalaludinii, and Blautia producta—formed a group that was termed “Block 5” (FIG. 8D, right panel). Blocks 1 and 5 exhibited collective similarity amongst each other; this group of strains was termed “SynCom15”—a 15-member group comprised of statistically interacting strains that were co-present in communities predicted to clear K. pneumoniae. In contrast to SynCom15, three other groups of strains were statistically inferred to be co-absent in communities predicted to clear K. pneumoniae. These groups were comprised of 7, 10, and 14 strains; these groups were termed “Block 2,” “Block 3,” and “Block 4” respectively (FIG. 8D, right panel; Supplementary Table 6G).

The inventors hypothesized that SynCom15 would be efficacious at clearing K. pneumoniae across different environments because it was predicted to contain the key, critical species for DMC function. The inventors built and tested SynCom15 as well as all other Blocks for their capacity to clear K. pneumoniae across three unrelated media conditions: BHIS, media created from the cecal extracts of germ-free (GF) mice, and media created from the cecal extracts of specific-pathogen-free (SPF) mice treated with broad spectrum antibiotics (Ab-treated SPF) (FIG. 8E, top panel; Example 3). As a comparator to SynCom15 and the other Blocks, the inventors also tested DMC46—the community that suppressed K. pneumoniae the most in BHIS media. Notably, the results clearly illustrated the environmental dependence of community efficacy. Blocks 2 and 3 were consistently ineffective at suppressing K. pneumoniae across environments while Blocks 1, 4 and 5 were able to suppress K. pneumoniae depending on the environment in which they were tested—Block 1 in BHIS and Blocks 4 and 5 in GF cecal extract. Thus, Blocks 1, 4 and 5 were conditionally effective. In contrast, the inventors found that DMC46 and SynCom15 suppressed K. pneumoniae across all three conditions and were therefore unconditionally effective. DMC46 cleared K. pneumoniae across all environments. SynCom15 suppressed K. pneumoniae five orders of magnitude in BHIS, cleared K. pneumoniae in GF cecal extracts, and suppressed K. pneumoniae greater than four orders of magnitude in Ab-SPF cecal extracts. (FIG. 8E, bottom panels; Supplementary Table 7).

Thus, the inventors' strategy of statistical inference performed on the RF model of community design defined SynCom15—a phylogenetically diverse 15-member community—that suppressed K. pneumoniae across diverse environmental contexts in a manner similar to DMC46—the community containing all 46 strains.

SynCom15 cleared K. pneumoniae in a pre-clinically relevant mouse model of infection: Because DMC46 and SynCom15 were unconditionally effective at clearing K. pneumoniae in vitro, the inventors sought to test the ability of both communities to clear K. pneumoniae in a more complex, clinically relevant environment. The inventors evaluated the efficacy of DMC46 and SynCom15 in a mouse model of infection. To mimic a clinically relevant scenario, the inventors did not use germ-free mice (mice without a microbiome). Rather, treated SPF mice with broad spectrum antibiotics to deplete their gut microbiota then infected them with K. pneumoniae—a sequence of events commonly encountered in patients who acquire MDR K. pneumoniae infection. Additionally, singly-housed mice were used to ensure that no sharing of microbes by coprophagia amongst animals would affect microbiome composition during and post-antibiotic treatment⁴⁰. Singly-housed antibiotic-treated SPF mice infected with K. pneumoniae MH258 were given either (i) saline (PBS), (ii) a heterologous whole stool transplant derived from mice (“Fecal Microbial Transplant”, FMT), (iii) Block 1, (iv) Block 2, (v) DMC46, or (vi) SynCom15 as interventions for three sequential days after infection (Example 3). Blocks 1 and 2 were given as bacterial communities that were either conditionally efficacious across in vitro conditions or unable to clear K. pneumoniae across any in vitro condition respectively. Fecal samples were collected and K. pneumoniae abundances were tracked through the course of the experiment by plating (FIG. 9A).

The inventors found that Block 1, and Block 2 did not suppress K. pneumoniae relative to saline. The FMT suppressed K. pneumoniae three orders of magnitude one day after the last gavage and up to six orders of magnitude from four days after the last gavage until the end of the experiment. DMC46 suppressed K. pneumoniae three orders of magnitude one day after the last gavage, four orders magnitude four days after the last gavage, and six orders of magnitude nine days after the last gavage. Thus, DMC46 was able to suppress K. pneumoniae but exhibited slow kinetics of response compared to the FMT. In contrast, SynCom15 rapidly suppressed K. pneumoniae, resulting in a reduction of abundance by five orders of magnitude one day after the last gavage. Additionally, SynCom15 cleared K. pneumoniae four days after the last gavage and maintained clearance through nine days after the last gavage (FIG. 9B; Supplementary Table 8). These results highlighted the rapid and sustained efficacy of SynCom15 in clearing K. pneumoniae in vivo as well as the utility of reducing the community size from the 46 strains defining DMC46 to the inferred key 15 strains defining SynCom15.

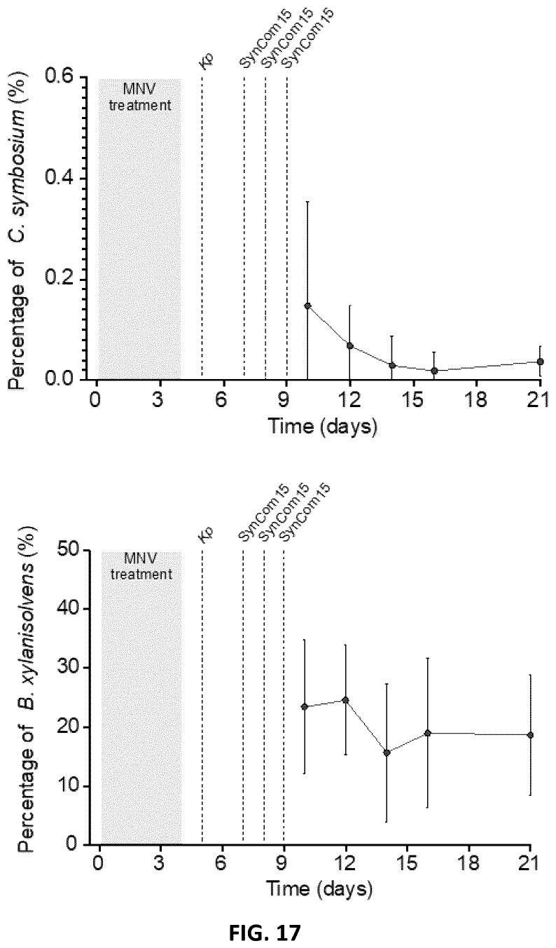

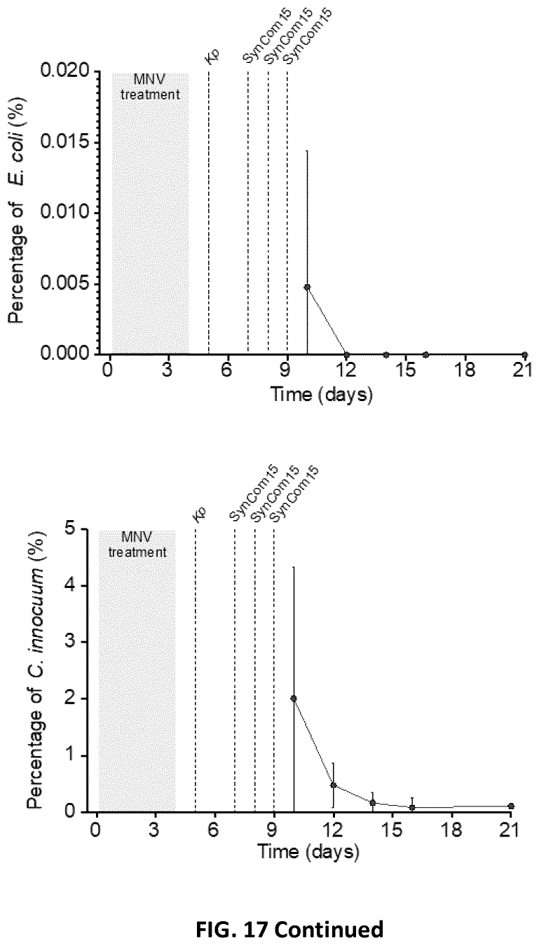

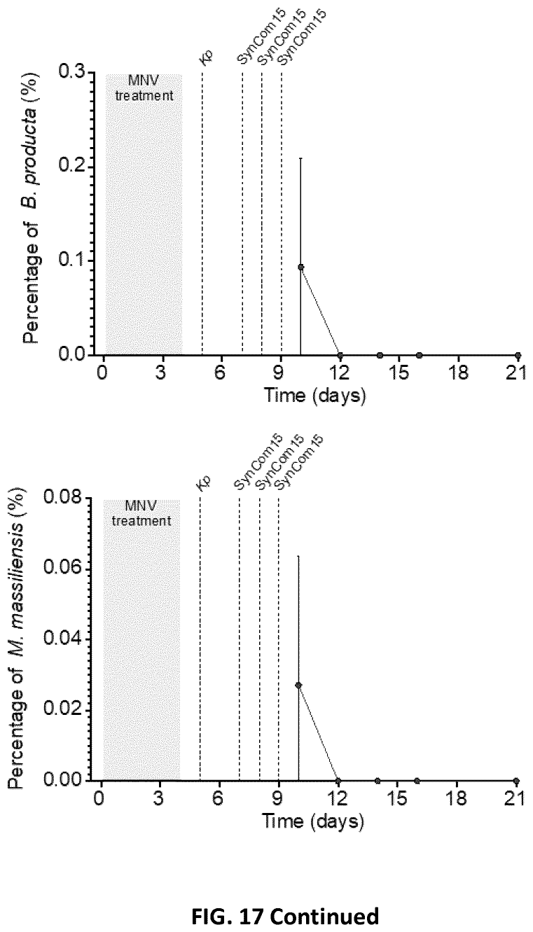

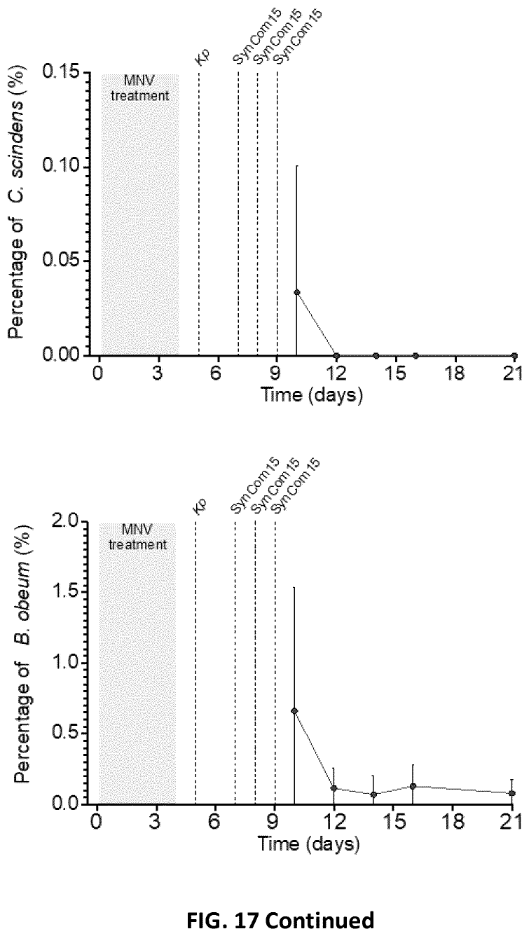

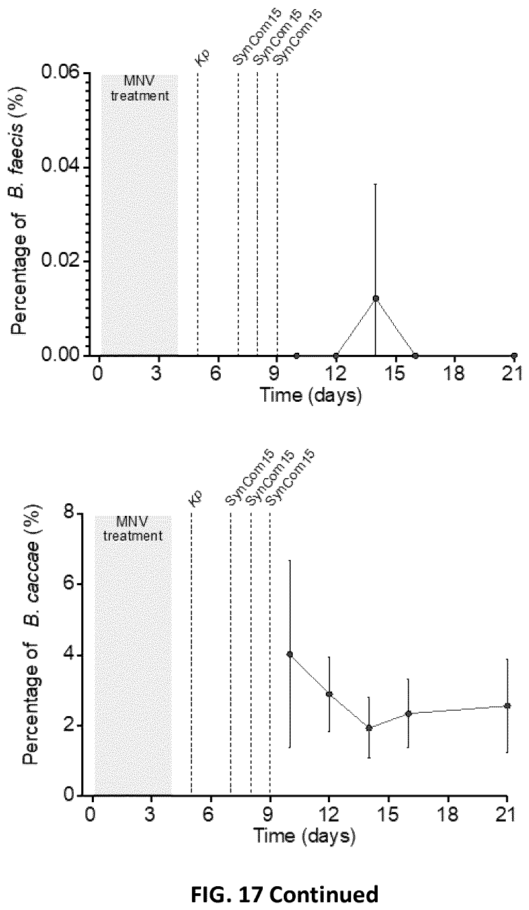

Taxonomic profiling of fecal samples procured through the experiment revealed that 10 of the 15 strains in SynCom15 engrafted in at least one of the mice within the cohort (FIG. 9C; Example 3). Dynamics of SynCom15 strains showed that 5 of the 10 strains were present at detectable fractional abundances throughout the course of the experiment—C. symbiosum, B. xylanisolvens, C. innocuum, B. obeum, and B. caccae (FIG. 17). Together, these results illustrated that the engraftment and strain dynamics of SynCom15 in mice did not follow obvious phylogenetic trends.

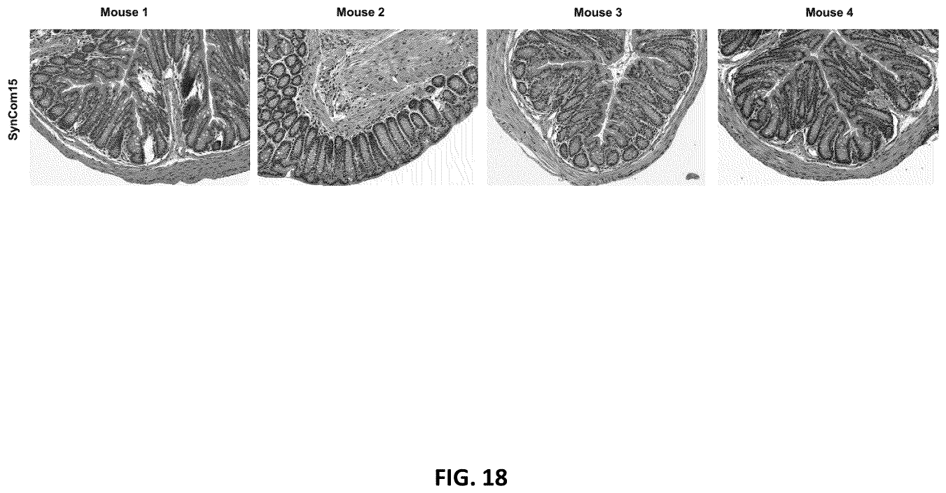

Dynamics of microbiota diversity and structure within the infected mice treated with SynCom15 mirrored that of the FMT and returned the state of the microbiota to that observed prior to antibiotic treatment (FIGS. 9D-9E; Supplementary Table 9A,B; Example 3). At the phylogenetic description of phylum, class or family, the inventors observed that treatment via FMT and SynCom15 resulted in similar ending configurations of the microbiota (FIG. 9F). However, at the genus-level description, the inventors observed differences between the ending microbiota configuration of mice treated with FMT or SynCom15. Treatment with FMT resulted in the return of Duncaniella and Paramuribaculum (genera belonging to the order Bacteroidales). Treatment with SynCom15 resulted in detectable presence of the genera Bacteroides, derived from the B. xylanisolvens strain in SynCom15, in addition to a bloom of Bifidobacterium (FIG. 9G and FIG. 17). These results illustrated that treatment with SynCom15 yielded a return to a diverse microbiota that resembles a more human-like signature despite being engrafted in mice. Histology of the mouse colon showed that SynCom15 was well tolerated as an intervention showing no evidence of inflammation or tissue insult (FIG. 18).

Collectively, these results demonstrated that SynCom15 successfully cleared K. pneumoniae in a pre-clinical mouse model of infection—a result consistent with the inventors' findings showing that SynCom15 is unconditionally effective across in vitro environments. Additionally, the inventors found that treatment with SynCom15 was safe from the standpoint of microbiota recovery and tissue injury. Together, these results pointed towards the therapeutic potential of SynCom15 for clearing K. pneumoniae from the gut.

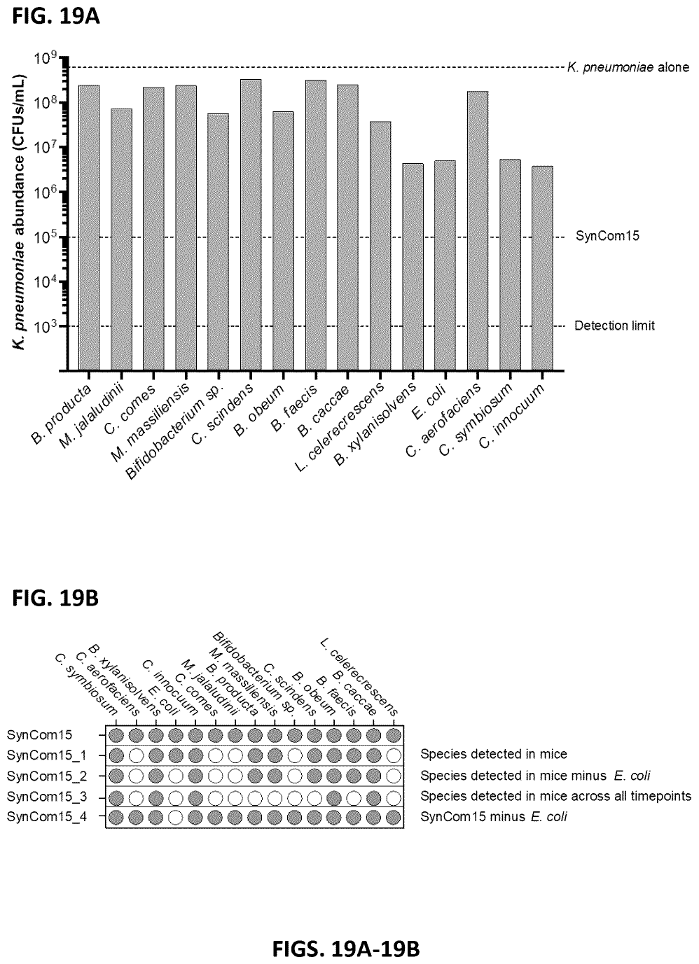

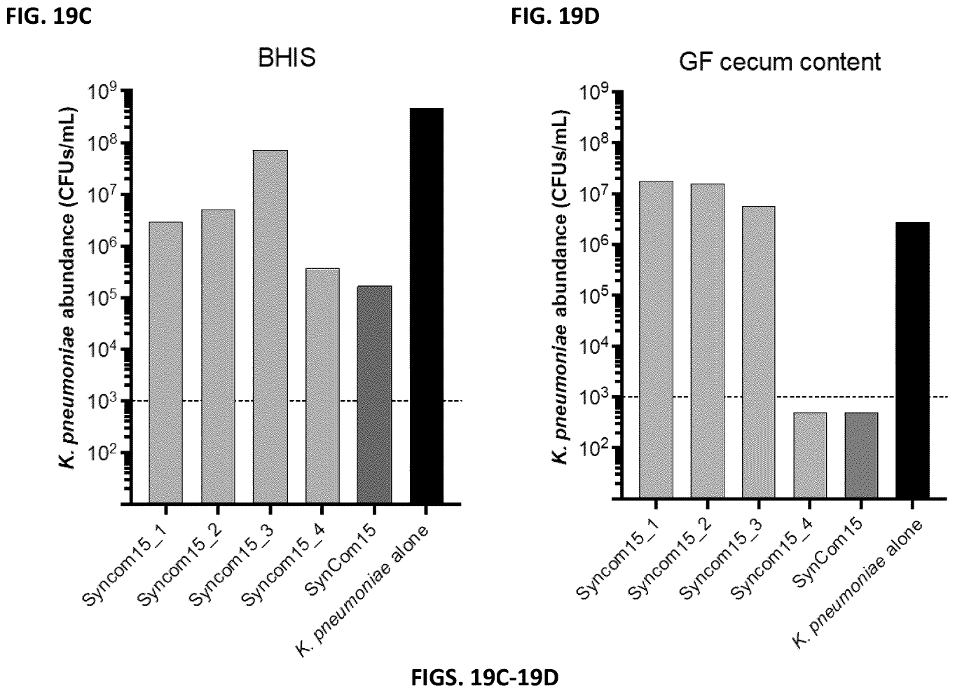

Compositional characterization of SynCom15: Given the safety and efficacy of SynCom15, the inventors sought to further characterize its compositional content. First, the inventors tested each strain of SynCom15 individually for its ability to suppress K. pneumoniae in BHIS. It was found that no individual strain suppresses K. pneumoniae greater than two orders of magnitude and eleven of the strains suppressed K. pneumoniae only up to one order of magnitude (FIG. 19A, Supplementary Table 10A). Moreover, the four strains that suppressed K. pneumoniae two orders of magnitude were found in Block 1, a Block that suppressed K. pneumoniae comparable to SynCom15 in BHIS but was less efficacious by several orders of magnitude in other environments without the addition of the other ten strains comprising SynCom15. Thus, including the eleven strains that have only a modest individual effect on suppressing K. pneumoniae in BHIS media was important for achieving the suppressive capacity of SynCom15 in other environments. These findings highlight the complex nature of the ability of SynCom15 to suppress K. pneumoniae across environments.

Next, the inventors interrogated whether data from the mouse experiments could inform which strains of SynCom15 were important for functionality. The inventors built two communities: (i) a community constituting strains that consistently engrafted the mice (10 species); and (ii) a community constituting strains that were consistently detected in mice across all timepoints (5 species) (FIG. 19B). The first community suppressed K. pneumoniae two orders of magnitude in BHIS and did not suppress K. pneumoniae in GF cecal extract media; the second community suppressed K. pneumoniae one order of magnitude in BHIS and did not suppress K. pneumoniae in GF cecal extract media (FIG. 19C-19D, Supplementary Table 10B). Thus, the inclusion of strains constituting SynCom15 that were not statistically detectable in the mouse fecal pellets was important for achieving the clearance of K. pneumoniae observed across environments.

Recent results have claimed the critical importance of E. coli in clearing K. pneumoniae²⁵. This motivated the inventors to test the importance of the E. coli strain for SynCom15. Therefore, the inventors built two more communities-SynCom15 without E. coli and the community comprised of strains that engrafted the mouse without E. coli (FIG. 19B). Removing E. coli from either community resulted in a decrease in K. pneumoniae suppression by just half an order of magnitude in BHIS and no difference in suppression in GF cecal extract media (FIG. 19C-19D, Supplementary Table 10B). Additionally, the inventors noted that the Block 1 community—a five-member community containing E. coli—was unable to suppress K. pneumoniae in mice more than a saline intervention at day 11 and day 16 post infection (FIG. 9B). Recent studies have also suggested augmenting E. coli with large, diverse communities to clear K. pneumoniae²⁵. The inventors' data provided a contrasted result: DMC46, a diverse community comprised of 46 strains, contained a strain of E. coli but was not as effective as SynCom15, comprised of 15 strains including the same E. coli strain, at suppressing K. pneumoniae in mice (FIG. 9B).

Collectively, these observations illustrated that the efficacy of SynCom15 as a community that suppressed K. pneumoniae across different environments could be solely ascribed to the presence of any single strain, including E. coli, or an obvious subset of strains gleaned from analysis of the mouse experiments. Moreover, coarse community descriptions, like community diversity for instance, did not provide an explanation for the results. In contrast, the findings highlighted the utility of evaluating community function through the inventors' statistical approach that considered emergent, and potentially non-obvious properties of the structure-function relationship for communities.

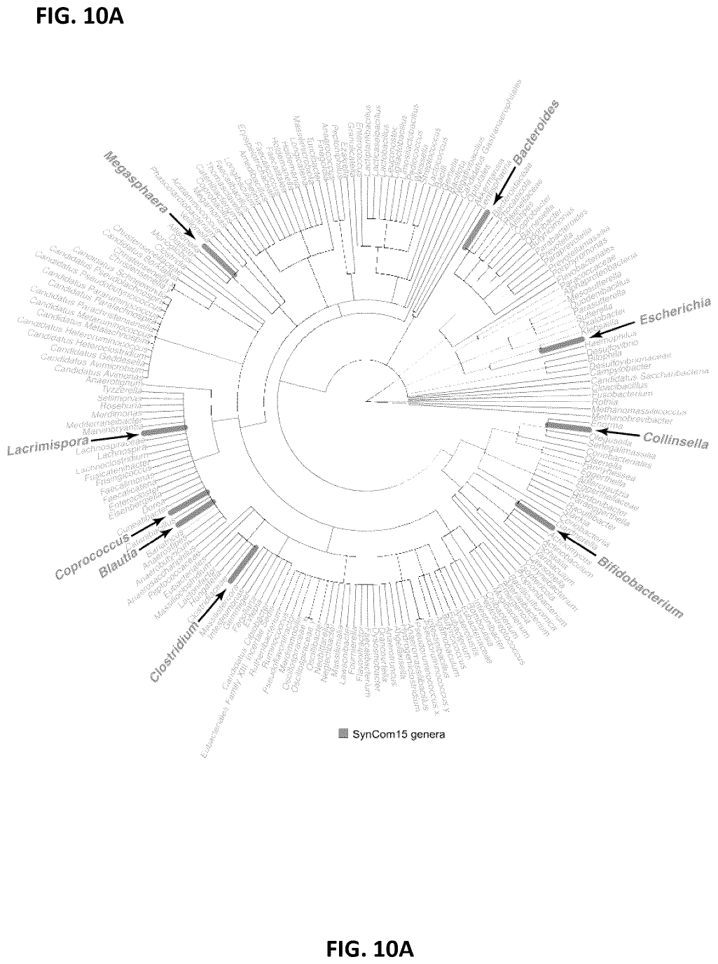

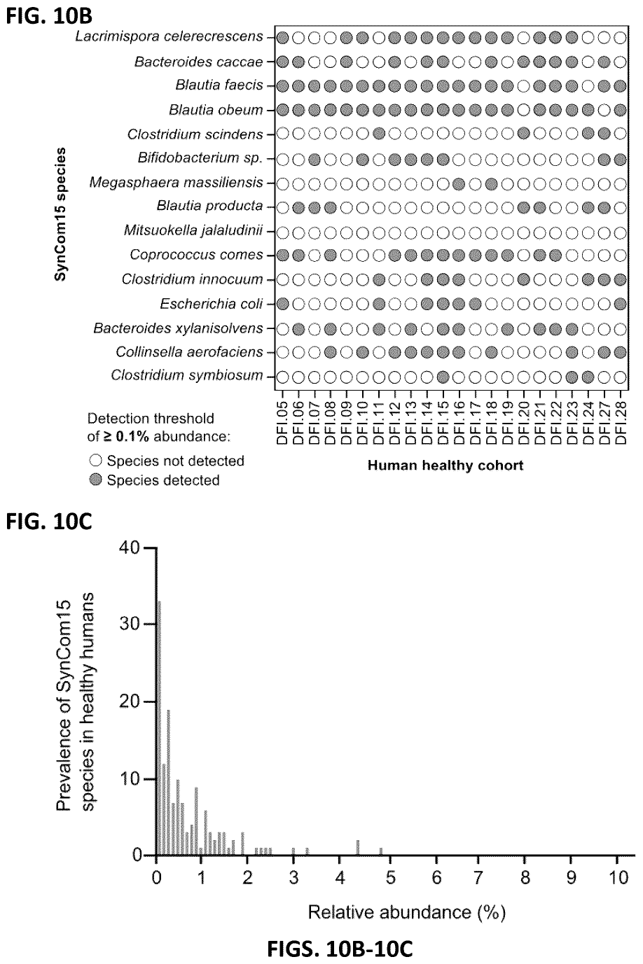

Comparison of SynCom15 with composition of healthy human fecal microbiomes: Next, the inventors explored the extent to which SynCom15 was represented across healthy humans who provided FMTs from which the strain bank was created. The inventors first interrogated the prevalence of the genera constituting SynCom15 in fecal samples from healthy donors, and found that the genera represented in SynCom15 reflected a diverse minority of the totality of genera observed across the set of healthy gut microbiomes (FIG. 10A). Next, the inventors interrogated the prevalence of the SynCom15 species across the fecal samples of the healthy donors (Example 3), and found that no healthy human microbiome contained more than eleven of the SynCom15 species above a fractional abundance of 0.1% (FIG. 10B; Supplementary Table 11). Moreover, the inventors found certain SynCom15 species to be remarkably sparse in their prevalence across donors. M. jalaludinii was not detectable in any donor; M. massiliensis was detectable in two donors; C. symbiosum in three donors; and C. scindens in four donors. Amongst strains that were most prevalent, B. obeum and B. faecis were detectable in 20 donors; L. celerecrescens in 14 donors; C. comes in 13 donors; B. caccae in 12 donors. Finally, the inventors interrogated the fractional abundance of SynCom15 species across the fecal samples of the 22 healthy donors, and found SynCom15 species were present at a relative abundance of less than 5% across all donors, with a majority of species being found at a relative abundance of less than 0.5% (FIG. 10C).

Together, these results illustrated two conclusions. First, the composition of SynCom15 was distinct from that found across healthy human gut microbiotas. This was either because SynCom15 does not exist in the healthy samples from the inventors' cohort or because several of SynCom15 strains are undetectable by the sequencing methods employed due to their low abundance. Second, the strains comprising SynCom15 were low prevalence and abundance amongst fecal samples of healthy donors. This result highlighted the power of generating and using broadly diverse strain banks for engineering synthetic bacterial communities as compared to strain banks reflecting the compositional abundance and prevalence distributions gleaned from analysis of natural human microbiomes.

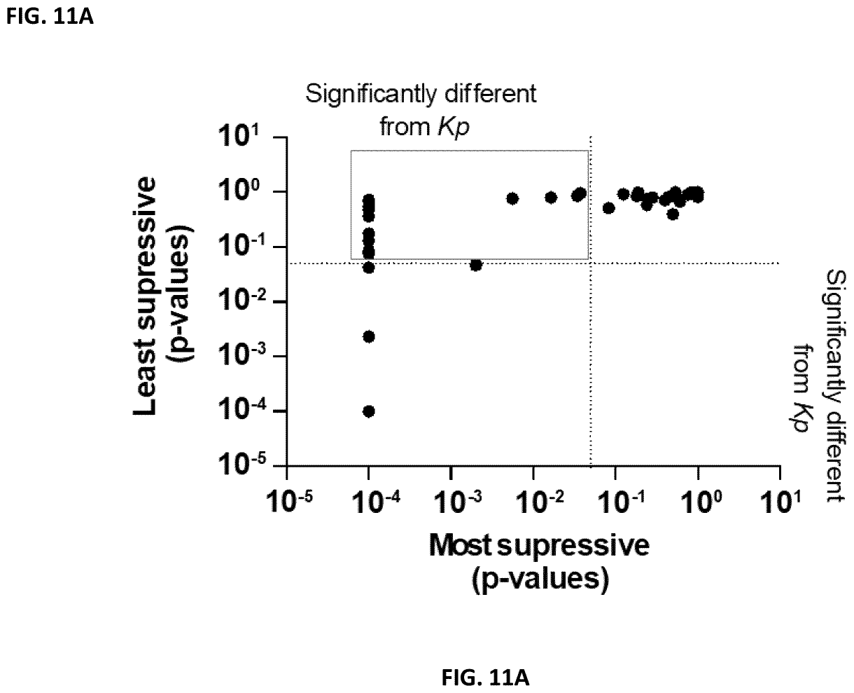

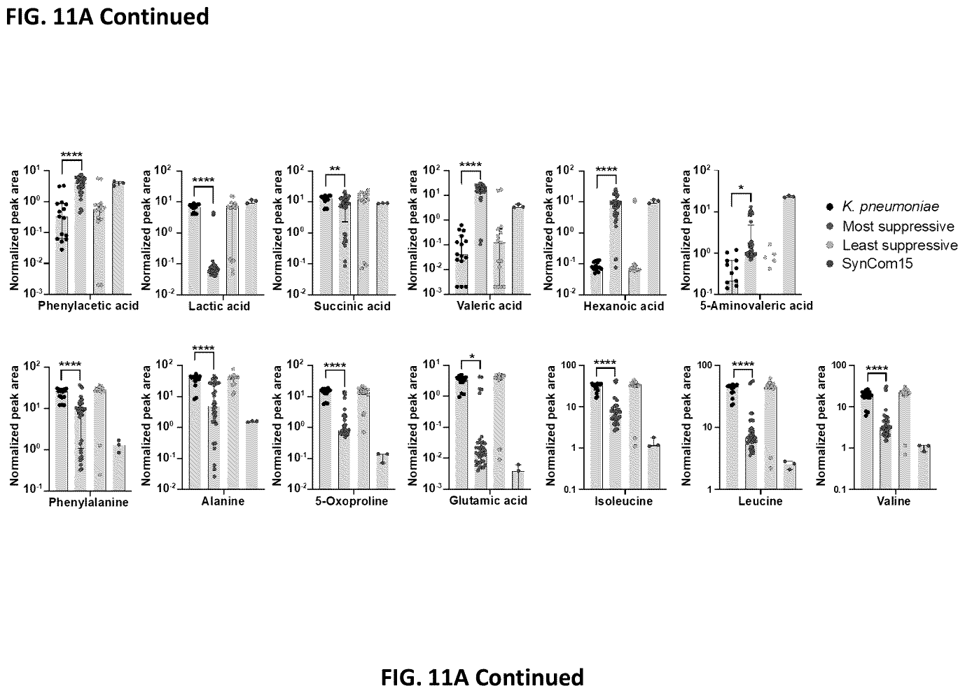

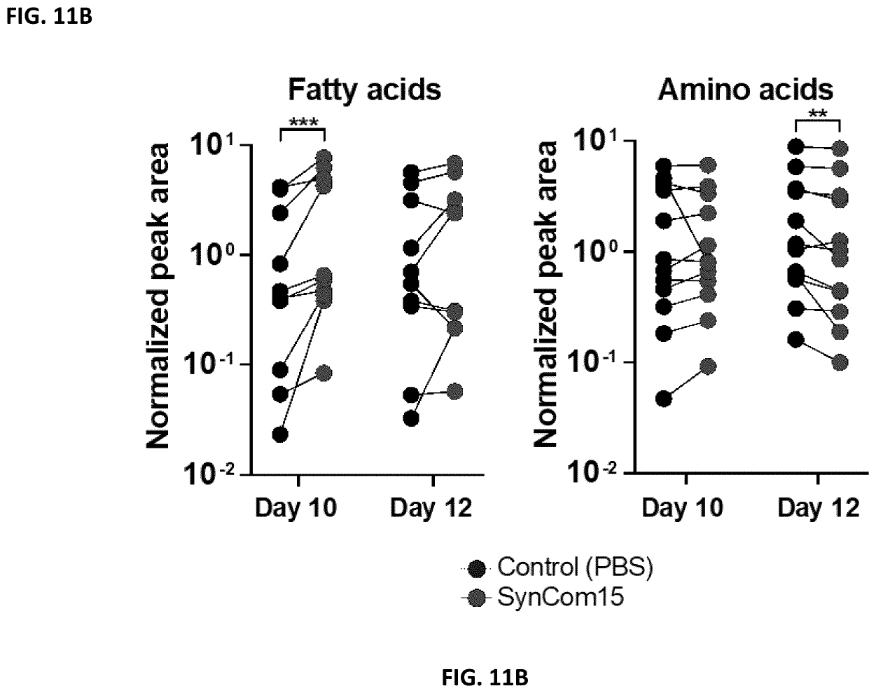

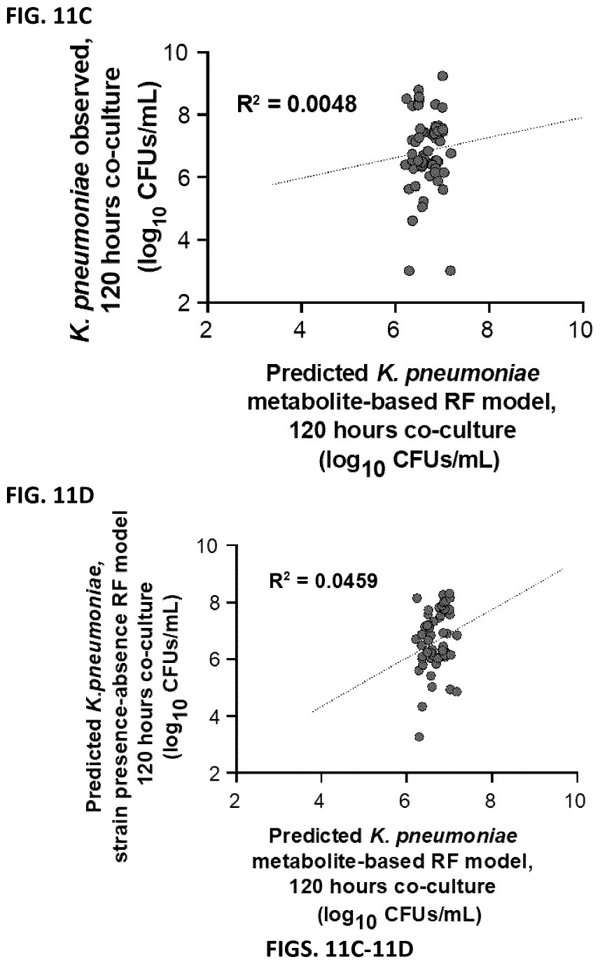

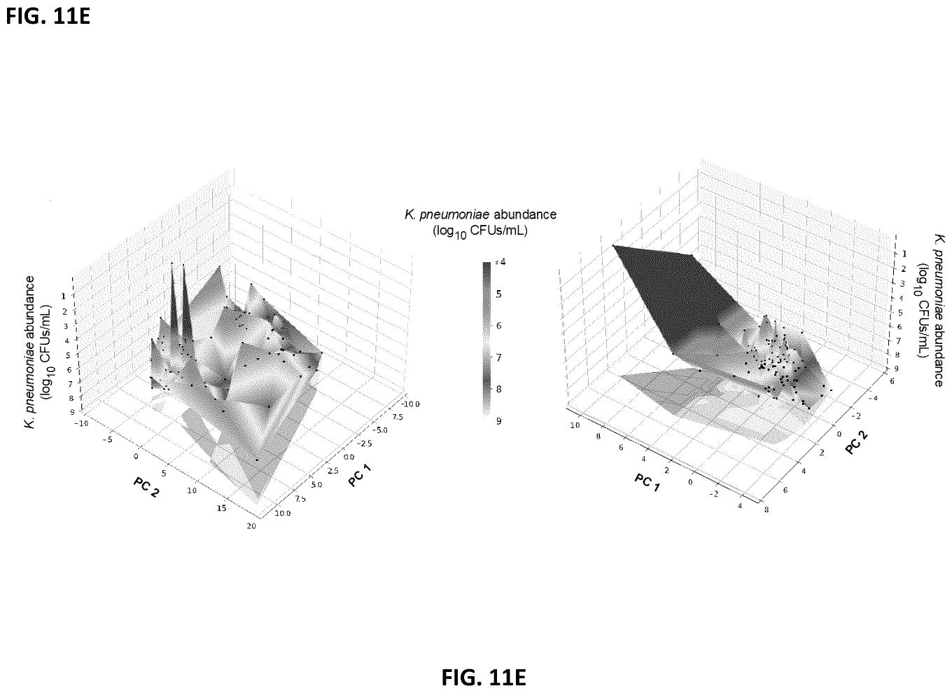

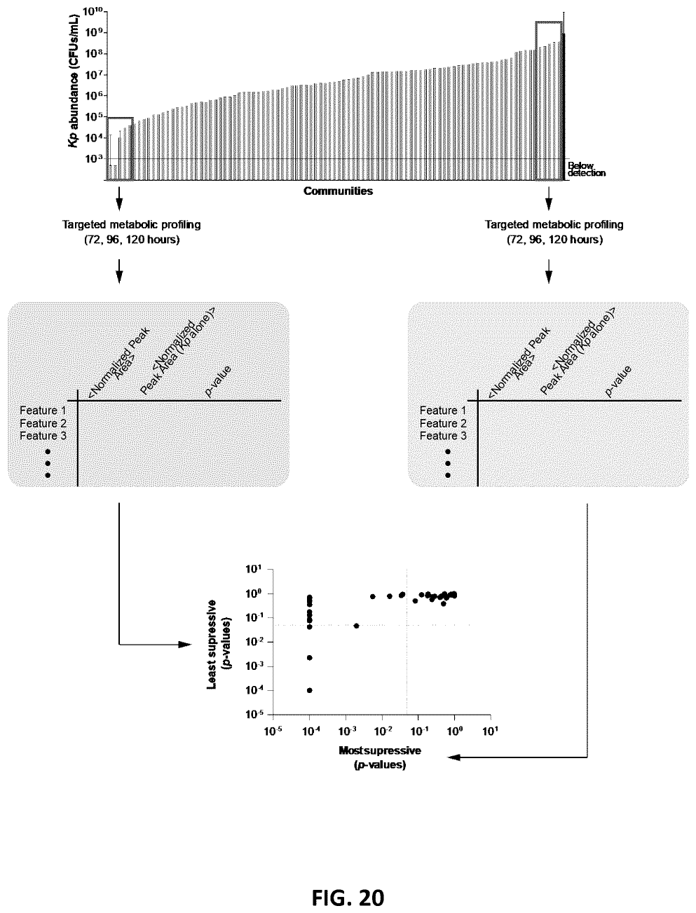

Community metabolism poorly predicted K. pneumoniae suppression: Engineering SynCom15 was based on statistical analysis of a model that described DMCs by their pattern strain presence-absence and their capacity to clear K. pneumoniae. Thus, the model was not constructed using any information about mechanism of action. Previous results have suggested the importance of media acidification and nutrient competition as mechanisms by which complex bacterial communities could suppress K. pneumoniae²⁻⁵²⁷. Therefore, the inventors compared the metabolic profiles of the five DMCs that suppressed K. pneumoniae the most against the five DMCs that suppressed K. pneumoniae the least amongst the 96 DMCs previously tested in BHIS (FIG. 11A, first panel; FIG. 20; Supplementary Table 12A; Example 3). The inventors analyzed the profile of 118 metabolites across the most and least suppressive DMCs after being co-cultured with K. pneumoniae for 72, 96, and 120 hours.

The metabolite patterns that distinguished DMCs that suppressed K. pneumoniae from those that did not centered around two metabolic axes: concentrations of fatty acids (FAs) with an emphasis on short-chain fatty acids and amino acids (Supplementary Table 12B). With respect to FAs, the most suppressive DMCs produced phenylacetic acid, valeric acid, hexanoic acid, and 5-aminovaleric acid and consumed lactic acid as well as succinic acid. With respect to amino acids, the most suppressive DMCs consumed either (i) amino acids with non-polar side chains (phenylalanine, alanine, isoleucine, leucine, valine) or (ii) glutamic acid and its associated derivative 5-oxoproline (FIG. 11A). Metabolic profiling of SynCom15 co-cultured with K. pneumoniae in BHIS revealed a similar trend. SynCom15 produced the same FAs as the most suppressive DMCs, but also produced lactic acid as opposed to consuming it. SynCom15 also consumed all the amino acids that the most suppressive DMCs consumed (FIG. 11A, Supplementary Table 12C). The inventors also performed metabolic profiling of fecal pellets collected from mice treated with either SynCom15 or saline in the experiment described in FIG. 9A. Consistent with the in vitro results, the inventors found a statistically significant increase in FA production on day 10 and amino acid depletion on day 12 in infected mice given SynCom15 (FIG. 11B, Supplementary Table 12D). The in vitro and in vivo results were in accordance with previously published studies demonstrating the importance of environmental acidification and nutrient competition as mechanisms by which MDR K. pneumoniae could be suppressed. Furthermore, these results pointed to metabolic axes that are shared between the function of SynCom15 in in vitro and in vivo conditions, suggesting a way that translatability of suppressive capacity across distinct environments could be manifested.

The inventors reasoned that if the mechanism of suppression was exclusively related to FA production and amino acid depletion, that they could build a generative statistical model of community design based on the metabolite profile of a large number of DMCs spanning a range of K. pneumoniae suppression. This would represent a more thorough test of the sufficiency of FA production and nutrient depletion to explain how DMCs clear K. pneumoniae. Thus, the inventors performed metabolic profiling of 81 DMCs that had been designed and tested in BHIS media for their capacity to suppress K. pneumoniae (Supplementary Table 13A). The inventors removed 15 DMCs from the analysis because they were poorly profiled across metabolite features. Metabolite profiles were measured at 72, 96, and 120 hours of co-culture with K. pneumoniae. The inventors also performed metabolic profiling of the 60 DMCs that previously served as the “out-of-sample” DMCs at 72, 96, and 120 hours of co-culture with K. pneumoniae (Supplementary Table 13B). The inventors trained and validated an RF model on the metabolic profiles of the 96 DMCs to predict K. pneumoniae abundance after 120 hours of co-culture (Example 3). The inventors then evaluated the capacity of the trained model to predict the K. pneumoniae abundance of the 60 “out-of-sample” DMCs after 120 hours of co-culture using their metabolic profile, and found that the RF model trained on metabolite profiles was a markedly poor predictor of the K. pneumoniae abundance of the 60 out-of-sample DMCs, attaining no predictive power with an r² value of 0.0048 (FIG. 11C, Supplementary Table 13C). Following this result, as expected the predictive capacity of the RF model built on metabolite profiles shared no similarity in predictive capacity with the RF model built on strain presence-absence of DMCs that was highly predictive of K. pneumoniae abundance (FIG. 11D).

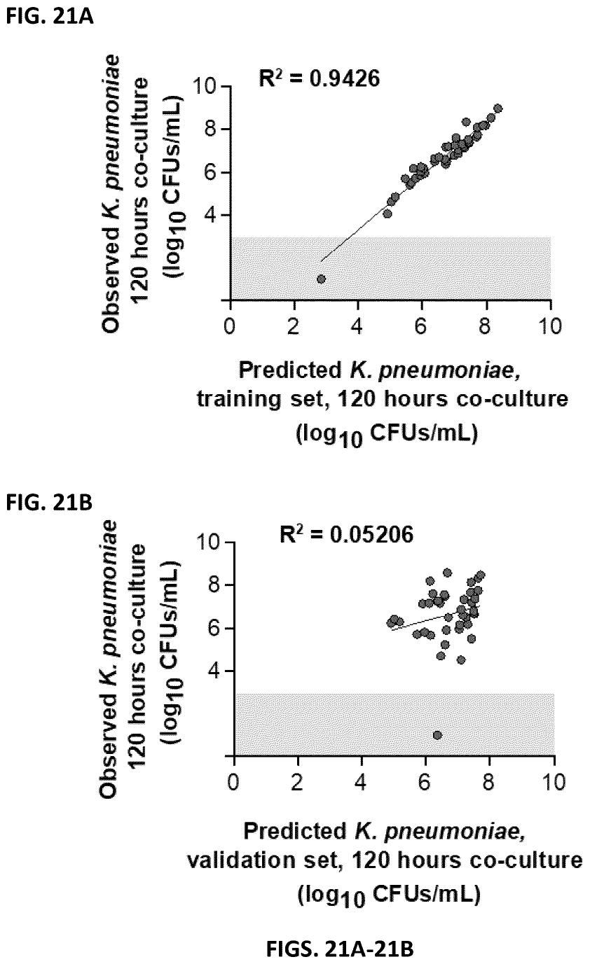

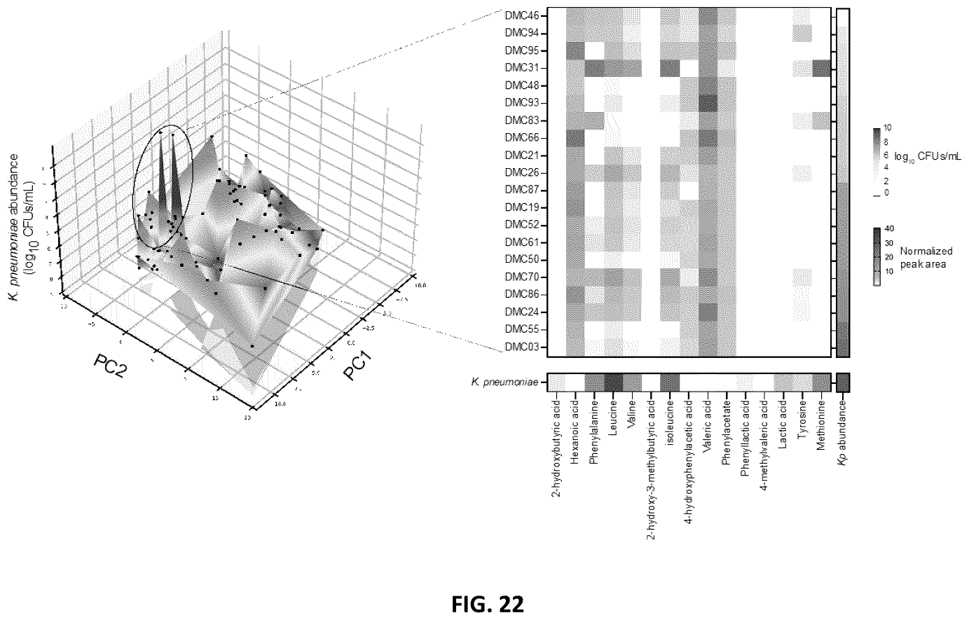

To understand why the metabolite profile of a community was a poor predictor of K. pneumoniae abundance, the inventors interrogated the structure of metabolite profiles across the DMCs used to train the model, and found that the neighborhood of metabolite space where there were DMCs that suppressed K. pneumoniae also contained poorly suppressive DMCs. That is, the metabolic landscape of DMCs was “rugged”—interspersed with peaks and valleys of suppressive capacity—rather than smooth (FIG. 11E, left panel; Supplementary Table 14A). This result demonstrated there was a degeneracy of different, unrelated metabolite profiles associated with clearing K. pneumoniae, resulting in a predictive model that was overfit to the training set and therefore unable to generate new functional communities (FIG. 21, Supplementary Table 14B). Consistent with this result, the inventors found that DMCs that were highly suppressive of K. pneumoniae shared similar metabolite profiles with DMCs that exhibited intermediate to low suppression of K. pneumoniae (FIG. 22, Supplementary Table 14C,D). In contrast, the landscape of DMCs defined by strain presence-absence was smooth, increasing in the capacity to suppress K. pneumoniae from negative to positive along the first principal component (FIG. 11E, right panel; Supplementary Table 14E). Thus, describing DMCs by their strain presence-absence defined a space that was co-linear with K. pneumoniae suppression thereby enabling learning an accurate statistical model of design. Collectively, these results showed that design based on a metabolic profile comprising the targeted panel of features (amino acids, aromatics, branch-chained fatty acids, indoles, phenolic aromatics, and short-chained fatty acids) may not be a reliable strategy for engineering communities that clear K. pneumoniae in a predictable manner. The inventors' findings highlighted the utility of considering the more coarse-grained description of strain presence-absence in creating generative models of community design.

Discussion: Using clearance of MDR K. pneumoniae as a target function, the inventors engineered a defined, sparse microbiome—SynComl5—that was complex, safe, efficacious, and distinct from natural human gut microbiome compositions using a statistical approach for community design. The results shed light on several notable findings.

First, merely designing genetically diverse communities did not guarantee creating functional communities. However, imposing the constraint of genetic diversity on the “Design” portion of DBTL was crucial for reducing the space of possible DMCs and was a particularly informative space for learning a generative statistical model. Indeed, extremely limited sampling (building and testing 96 out of the immense number of possible DMCs) was sufficient to converge on an accurate model of design in vitro. These results suggest a deep connection between the phylogenies of strains and the collective functions encoded by microbial communities, opening the possibility of phylogenetic-based “bottom-up” design. The development of emerging methods for parametrizing functional differences amongst strain-level variants through considering their evolutionary history across the bacterial tree-of-life will be useful for testing this idea in the future⁴¹.

Second, accurately translating microbiome function from specific in vitro settings to other in vitro and in vivo environments has historically been a significant challenge. The inventors' data showed that the generative model resulting from DBTL+ was insufficient for translating community function across different environments. However, the constraints of the model were sufficient for engineering a microbiome—SynCom15—that successfully translated function across environments. To understand why this may be, the inventors drew a parallel to learning theory in computer science. A well-known problem in building models is creating statistical representations that are “overfit” to training environments. Analogously, performing DBTL+ in a single environment, like BHIS, resulted in a generative model that was “overfit” to the environment in which DMCs were tested. A key insight that resulted from the inventors' work was that learning the constraints on the model in a single environment enabled generalization of function to new environments (e.g. cecal extract medias and SPF-infected mice). This finding was consistent with emerging evidence suggesting that a way that the evolutionary process can generate adaptable systems is not selecting for individual systems that function per se, but by selecting for underlying structural regularities amongst ensembles of systems that function⁴². Using structural regularities across functional systems as a criteria for design may create new systems where variance in a core function is far lower than the variance encountered across different environments, thereby enabling translatability. By inferring conserved statistical patterns across thousands of DMCs that were predicted to highly suppress K. pneumoniae, the inventors' approach of statistical inference may be an analytical manifestation of this principle.

Third, the inventors' results demonstrated how using metabolite information spanning previously appreciated mechanisms by which K. pneumoniae could be suppressed resulted in a poor generative model of community design. These findings suggested that likely, there are a myriad of mechanisms by which the clearance of K. pneumoniae can be realized. These mechanisms may be included in metabolic panels encompassing a broader set of features than those found by the inventors or revealed by other “-omics”-based panels that are becoming more common in microbiome studies such as proteomics or transcriptomics. While future efforts aimed at collecting such large datasets may be warranted to further elucidate mechanisms of K. pneumoniae suppression and clearance, the inventors' results demonstrated that such information is unnecessary for creating generative models of community design.

Fourth, SynCom15 was more efficacious at suppressing K. pneumoniae in mice compared to DMC46—a 46-member community that contained the 15 strains defining SynCom15. This result highlights the functional power of defined small bacterial communities in contrast to recent studies advocating engineering large communities spanning 50 to greater than 100 strains^{10,25}. In addition to the gain in clearance capacity of K. pneumoniae, the inventors stressed that the ability to engineer sparse, functional bacterial communities is a tremendous advantage from a manufacturing and regulatory standpoint for creating therapeutic consortia for clinical use⁴³. Using DBTL+ coupled with statistical inference could be a procedure for achieving this goal in an efficient manner.

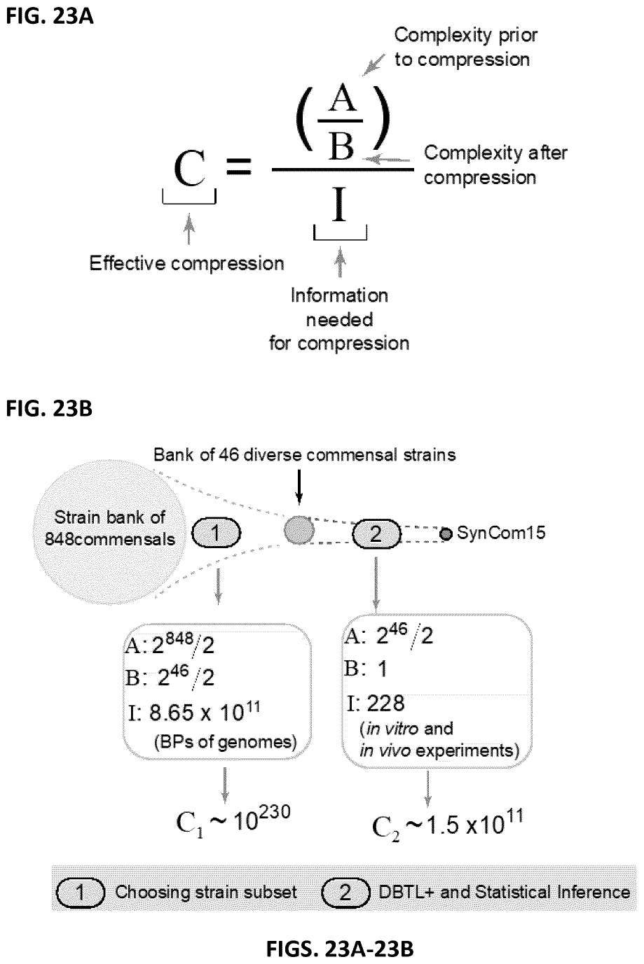

Given previous studies highlighting the immense complexity between structure-function relationships in microbial ecosystems, it may be expected that lots of high-content measurements or complex computational models trained on many parameters are necessary pre-requisites for deriving generative design principles of functional microbial communities^{10,14,16,44,45}. Consistent with this notion, existing efforts have utilized several different avenues of knowledge to inform community design. These include: (i) sophisticated modeling of dynamical interactions between microbes and of the community as a whole, (ii) detailed mechanistic knowledge of microbial interactions or mechanisms underlying a desired target function, (iii) knowledge about the presence or absence of specific biological pathways encoded within bacterial genomes comprising communities, (iv) knowledge about existing human microbiome composition and structure, or (v) using the existence of natural communities with desired functional traits (e.g. a fecal sample that resists colonization of gut pathogens) to reduce community size by serial iterative rounds of screening^{10-13,15,22,23,25,27,46-49}. The inventors' results paint a substantially different picture. The inventors found that merely the pattern of strain presence-absence coupled with the performance of a remarkably small number of designed diverse communities was sufficient to: (i) derive statistical generative models of community design de novo using relatively simple learning algorithms (e.g. an RF machine-learning model) and (ii) engineer communities whose functional capacity was translatable into new and markedly more complex environments. In analogy to the evaluation of computational algorithms, the inventors' two-step approach—(i) using proteome content to reduce the strain bank from 848 to 46 strains and (ii) implementing DBTL+ with statistical inference—was substantially compressive, able to navigate a remarkably high-dimensional space to converge on SynCom15 with little information relative to the starting combinatorial complexity (Supplementary Discussion, FIG. 23). A likely driving force behind these results for the target function of K. pneumoniae suppression was that in contrast to the apparent complexity of microbial ecosystems, profoundly low-dimensional representations of structure-function relationships exist and can be discovered in a facile manner by placing statistical patterns of phenomenology before biological understanding—an emerging viewpoint that has been the subject of some recent efforts in microbiome studies and has rapidly found immense success in the form of deep-learning models at other scales of biology, namely synthetic protein design^{15,20,21,50-54}. Following this, the inventors noted that the present approach did not consider mechanisms of action at any scale nor compositional information about natural microbiomes and their associated functions. As the test (“T”) module in the inventors' DBTL+ framework could be swapped out for theoretically any function with an assay, the inventors posed that their approach could, in principle, enable the statistical design of functional microbial communities distinct from those found in nature and the pursuit of synthetic ecology more broadly.

Supplementary Discussion. Assessing the compressive power of the inventors' approach: The process by which the inventors converged on SynCom15 as a community that cleared K. pneumoniae involved (i) reducing the complexity of the strain bank from 848 to 46 diverse strains and (ii) performing DBTL+ in BHIS and statistical inference with experimental validation in vitro and in vivo. Conceptualizing this two-step process as an algorithm, the inventors sought to compute the equivalent of a “compression” for converging on a single functional complex community from a bank of 848 strains. In evaluating computational algorithms, compression is a measure of data complexity prior to compression relative to after compression. As the inventors' process took into account biological information in the form of bacterial genome sequences and experiments, the inventors normalized the compression ratio by the amount of information needed to perform the compression. Therefore, defined an “effective compression” as

$$C = \frac{( \frac{A}{B} )}{I} \qquad ( 1 )$$

where C is the effective compression of a process, A is the complexity of data prior to compression, B is the complexity of data after compression, and I is the information needed for compression from A to B (FIG. 23A).

For our first step, the inventors reduced the strain bank from 848 strains to 46 strains representative of the full phylogenetic diversity by genome sequencing each of the 848 strains, annotating each genome by their gene content, and performing dimension-reduction via a UMAP analysis. Therefore, the total complexity prior to compression was 2⁸⁴⁸/2, the total complexity after compression was 2⁴⁶/2, and the information needed to be collected for compression were all base pairs of the 848 commensal strains (8.65×10¹¹ base pairs). Considering these values, the effective compression of the first step was ˜10²³—a substantial compression driven by the sizeable drop in complexity of the strain bank (FIG. 23B). For the second step, the inventors used the diversity of the 46 strains to create 96 DMCs, 60 “out-of-sample” DMCs, the inventors learned an RF model and performed statistical inference to derive SynCom15; and performed 72 more experiments to show that SynCom15 could generally clear K. pneumoniae. Therefore, the total complexity prior to compression was 2⁴⁶/2, the total complexity after compression was 1 (SynCom15), and the information needed to be collected for compression was 228 total experiments. Considering these values, the effective compression for the second step was ˜10¹¹ (FIG. 23B).

Collectively, this analysis showed that despite the apparently immense amount of data reflected in the whole genome sequences of 848 bacterial strains, this complexity was offset by many orders of magnitude through the inventors' approach of reducing combinatorial dimensionality by diversity-based design and DBTL+ with statistical inference. That is, the amount of compressive information held by the set of bacterial genomes was a markedly small fraction of the compressive information encoded by the two-step process developed by the inventors. The inventors comment on why their approach may be achieving a high compressive power in the Discussion.

Data Availability: The datasets generated in this example are available within Supplementary Tables. Metagenomic data generated from profiling of human fecal microbiomes used in this example are publicly available on NCBI under BioProject ID PRJNA838648. 16S data generated from mouse experiments used in this example are publicly available on NCBI under BioProject ID PRJNA1074807. Raw data files associated with metabolomic data used in this study can be found on MassIVEW repository MSV000094183.

Code Availability: All code was written in either Python or R; code for all analysis will be found on Github (https://github.com/aramanlab/Oliveira_et_al_2024).

Supplementary Data: The alignment of 848 gut commensal strains annotated by Prokka annotations can be found in dryad.

The contents of these examples, e.g., supplementary tables, are described in Oliveira R., et al., Statistical design of a synthetic microbiome that clears a multi-drug resistant gut pathogen. BioRxiv, Feb. 29, 2024, doi: https://doi.org/10.1101/2024.02.28.582635, which is incorporated herein by reference in its entirety.

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references (including those listed above), scientific articles, patent publications, and any other documents cited herein are hereby incorporated by reference for the substance of their disclosure.

### REFERENCES

1. Nayfach, S. et al. A genomic catalog of Earth's microbiomes. Nat. Biotechnol. 39, 499-509 (2021).
2. Sunagawa, S. et al. Tara Oceans: towards global ocean ecosystems biology. Nat. Rev. Microbiol. 18, 428-445 (2020).
3. Integrative HMP (iHMP) Research Network Consortium. The Integrative Human Microbiome Project. Nature 569, 641-648 (2019).
4. Becks, L., Hilker, F. M., Malchow, H., JUrgens, K. & Arndt, H. Experimental demonstration of chaos in a microbial food web. Nature 435, 1226-1229 (2005).
5. Zomorrodi, A. R. & Segre, D. Synthetic Ecology of Microbes: Mathematical Models and Applications. J. Mol. Biol. 428, 837-861 (2016).
6. Lawson, C. E. et al. Common principles and best practices for engineering microbiomes. Nat. Rev. Microbiol. 17, 725-741 (2019).
7. Vrancken, G., Gregory, A. C., Huys, G. R. B., Faust, K. & Raes, J. Synthetic ecology of the human gut microbiota. Nat. Rev. Microbiol. 17, 754-763 (2019).
8. Alivisatos, A. P. et al. MICROBIOME. A unified initiative to harness Earth's microbiomes. Science 350, 507-508 (2015).
9. Dubilier, N., McFall-Ngai, M. & Zhao, L. Microbiology: Create a global microbiome effort. Nature Publishing Group UK http://dx.doi.org/10.1038/526631a (2015) doi:10.1038/526631a.
10. Cheng, A. G. et al. Design, construction, and in vivo augmentation of a complex gut microbiome. Cell 185, 3617-3636.e19 (2022).
11. Atarashi, K. et al. Treg induction by a rationally selected mixture of Clostridia strains from the human microbiota. Nature 500, 232-236 (2013).
12. Caballero, S. et al. Cooperating Commensals Restore Colonization Resistance to Vancomycin-Resistant Enterococcus faecium. Cell Host Microbe 21, 592-602.e4 (2017).
13. Clark, R. L. et al. Design of synthetic human gut microbiome assembly and butyrate production. Nat. Commun. 12, 3254 (2021).
14. Faith, J. J., Ahern, P. P., Ridaura, V. K., Cheng, J. & Gordon, J. I. Identifying gut microbe-host phenotype relationships using combinatorial communities in gnotobiotic mice. Sci. Transl. Med. 6, 220ra11 (2014).
15. Raman, A. S. et al. A sparse covarying unit that describes healthy and impaired human gut microbiota development. Science 365, (2019).
16. Chang, C.-Y., Bajić, D., Vila, J. C. C., Estrela, S. & Sanchez, A. Emergent coexistence in multispecies microbial communities. Science 381, 343-348 (2023).
17. Faust, K. & Raes, J. Microbial interactions: from networks to models. Nat. Rev. Microbiol. 10, 538-550 (2012).
18. Hernindez Medina, R. et al. Machine learning and deep learning applications in microbiome research. ISME Commun 2, 98 (2022).
19. Lawson, C. E. Retooling Microbiome Engineering for a Sustainable Future. mSystems e0092521 (2021).
20. Skwara, A. et al. Statistically learning the functional landscape of microbial communities. Nat Ecol Evol 7, 1823-1833 (2023).
21. Gowda, K., Ping, D., Mani, M. & Kuehn, S. Genomic structure predicts metabolite dynamics in microbial communities. Cell 185, 530-546.e25 (2022).
22. Baranwal, M. et al. Recurrent neural networks enable design of multifunctional synthetic human gut microbiome dynamics. Elife 11, (2022).
23. Stein, R. R. et al. Computer-guided design of optimal microbial consortia for immune system modulation. Elife 7, (2018).
24. Prioritization of pathogens to guide discovery, research and development of new antibiotics for drug-resistant bacterial infections, including tuberculosis. (World Health Organization, 2019).
25. Spragge, F. et al. Microbiome diversity protects against pathogens by nutrient blocking. Science 382, eadj3502 (2023).
26. Sorbara, M. T. et al. Inhibiting antibiotic-resistant Enterobacteriaceae by microbiota-mediated intracellular acidification. J. Exp. Med. 216, 84-98 (2019).
27. Osbelt, L. et al. Klebsiella oxytoca causes colonization resistance against multidrug-resistant K. pneumoniae in the gut via cooperative carbohydrate competition. Cell Host Microbe 29, 1663-1679.e7 (2021).
28. Kuby, M. J. Programming models for facility dispersion: The p-dispersion and maxisum dispersion problems. Geogr. Anal. 19, 315-329 (1987).
29. Erkut, E. The discrete p-dispersion problem. Eur. J. Oper. Res. 46, 48-60 (1990).
30. Drezner, Z. & Erkut, E. Solving the Continuous p-Dispersion Problem Using Non-Linear Programming. J. Oper. Res. Soc. 46, 516-520 (1995).
31. Xiong, H. et al. Distinct Contributions of Neutrophils and CCR2+ Monocytes to Pulmonary Clearance of Different Klebsiella pneumoniae Strains. Infect. Immun. 83, 3418-3427 (2015).
32. Breiman, L. Random Forests. Mach. Learn. 45, 5-32 (2001).
33. Liaw, A. & Wiener, M. Classification and Regression by randomForest. (2007).
34. Plerou, V. et al. Random matrix approach to cross correlations in financial data. Phys. Rev. E Stat. Nonlin. Soft Matter Phys. 65, 066126 (2002).
35. Halabi, N., Rivoire, O., Leibler, S. & Ranganathan, R. Protein sectors: evolutionary units of three-dimensional structure. Cell 138, 774-786 (2009).
36. Rivoire, O., Reynolds, K. A. & Ranganathan, R. Evolution-Based Functional Decomposition of Proteins. PLoS Comput. Biol. 12, e1004817 (2016).
37. Lutsiv, T. et al. Compositional Changes of the High-Fat Diet-Induced Gut Microbiota upon Consumption of Common Pulses. Nutrients 13, (2021).
38. Jia, J. et al. Conserved Covarying Gut Microbial Network in Preterm Infants and Childhood Growth During the First 5 Years of Life: A Prospective Cohort Study. Am. J. Clin. Nutr. 118, 561-571 (2023).
39. Dahirel, V. et al. Coordinate linkage of HIV evolution reveals regions of immunological vulnerability. Proc. Natl. Acad. Sci. U.S.A 108, 11530-11535 (2011).
40. Oliveira, R. A. et al. Klebsiella michiganensis transmission enhances resistance to Enterobacteriaceae gut invasion by nutrition competition. Nat Microbiol 5, 630-641 (2020).
41. Doran, B. A. et al. An evolution-based framework for describing human gut bacteria. bioRxiv (2023) doi:10.1101/2023.12.04.569969.
42. Kouvaris, K., Clune, J., Kounios, L., Brede, M. & Watson, R. A. How evolution learns to generalise: Using the principles of learning theory to understand the evolution of developmental organisation. PLoS Comput. Biol. 13, e1005358 (2017).
43. Oliveira, R. A. & Pamer, E. G. Assembling symbiotic bacterial species into live therapeutic consortia that reconstitute microbiome functions. Cell Host Microbe 31, 472-484 (2023).
44. Wang, M. et al. Strain dropouts reveal interactions that govern the metabolic output of the gut microbiome. Cell 186, 2839-2852.e21 (2023).
45. Diener, C. & Gibbons, S. M. More is Different: Metabolic Modeling of Diverse Microbial Communities. mSystems 8, e0127022 (2023).
46. Winkler, E. S. et al. The Intestinal Microbiome Restricts Alphavirus Infection and Dissemination through a Bile Acid-Type I IFN Signaling Axis. Cell 182, 901-918.e18 (2020).
47. Hromada, S. et al. Negative interactions determine Clostridioides difficile growth in synthetic human gut communities. Mol. Syst. Biol. 17, e10355 (2021).
48. Venturelli, O. S. et al. Deciphering microbial interactions in synthetic human gut microbiome communities. Mol. Syst. Biol. 14, e8157 (2018).
49. Connors, B. M. et al. Control points for design of taxonomic composition in synthetic human gut communities. Cell Systems 14, 1044-1058.e13 (2023).
50. Russ, W. P. et al. An evolution-based model for designing chorismate mutase enzymes. Science 369, 440-445 (2020).
51. Yeh, A. H.-W. et al. De novo design of luciferases using deep learning. Nature 614, 774-780 (2023).
52. Anishchenko, I. et al. De novo protein design by deep network hallucination. Nature 600, 547-552 (2021).
53. Wang, J. et al. Scaffolding protein functional sites using deep learning. Science 377, 387-394 (2022).
54. Watson, J. L. et al. De novo design of protein structure and function with RFdiffusion. Nature 620, 1089-1100 (2023).
55. Li, W. & Godzik, A. Cd-hit: a fast program for clustering and comparing large sets of protein or nucleotide sequences. Bioinformatics 22, 1658-1659 (2006).
56. Huang, Y., Niu, B., Gao, Y., Fu, L. & Li, W. CD-HIT Suite: a web server for clustering and comparing biological sequences. Bioinformatics 26, 680-682 (2010).
57. Bolger, A. M., Lohse, M. & Usadel, B. Trimmomatic: a flexible trimmer for Illumina sequence data. Bioinformatics 30, 2114-2120 (2014).
58. Prjibelski, A., Antipov, D., Meleshko, D., Lapidus, A. & Korobeynikov, A. Using SPAdes De Novo Assembler. Curr. Protoc. Bioinformatics 70, e102 (2020).
59. Wood, D. E., Lu, J. & Langmead, B. Improved metagenomic analysis with Kraken 2. Genome Biol. 20, 257 (2019).
60. Camacho, C. et al. BLAST+: architecture and applications. BMC Bioinformatics 10, 421 (2009).
61. O'Leary, N. A. et al. Reference sequence (RefSeq) database at NCBI: current status, taxonomic expansion, and functional annotation. Nucleic Acids Res. 44, D733-45 (2016).
62. Chaumeil, P.-A., Mussig, A. J., Hugenholtz, P. & Parks, D. H. GTDB-Tk: a toolkit to classify genomes with the Genome Taxonomy Database. Bioinformatics 36, 1925-1927 (2019).
63. Blanco-Míguez, A. et al. Extending and improving metagenomic taxonomic profiling with uncharacterized species using MetaPhlAn 4. Nat. Biotechnol. 41, 1633-1644 (2023).
64. Chamberlain, S. A. & Szöcs, E. taxize: taxonomic search and retrieval in R. F1000Res. 2, 191 (2013).
65. Odenwald, M. A. et al. Bifidobacteria metabolize lactulose to optimize gut metabolites and prevent systemic infection in patients with liver disease. Nat Microbiol 8, 2033-2049 (2023).
66. Callahan, B. J. et al. DADA2: High-resolution sample inference from Illumina amplicon data. Nat. Methods 13, 581-583 (2016).
67. Wang, Q., Garrity, G. M., Tiedje, J. M. & Cole, J. R. Naive Bayesian classifier for rapid assignment of rRNA sequences into the new bacterial taxonomy. Appl. Environ. Microbiol. 73, 5261-5267 (2007).
68. Parks, D. H. et al. GTDB: an ongoing census of bacterial and archaeal diversity through a phylogenetically consistent, rank normalized and complete genome-based taxonomy. Nucleic Acids Res. 50, D785-D794 (2022).
69. Haak, B. W. et al. Impact of gut colonization with butyrate-producing microbiota on respiratory viral infection following allo-HCT. Blood 131, 2978-2986 (2018).

1. A composition comprising a bacterial population comprising at least one bacterial strain, wherein the bacterial strain(s) is/are a Clostridium innocuum strain, a Clostridium symbiosum strain, a Collinsella aerofaciens strain, an Escherichia coli strain, a Bacteroides xylanisolvens strain, a Lacrimispora celerecrescens strain (or Clostridium celerecrescens strain), a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, a Blautia producta strain, or any combination thereof.

2. The composition of claim 1, wherein the Clostridium innocuum strain comprises Clostridium innocuum strain MSK.7.7, the Clostridium symbiosum strain comprises Clostridium symbiosum strain MSK.7.21, the Collinsella aerofaciens strain comprises Collinsella aerofaciens strain MSK.1.10, the Escherichia coli strain comprises Escherichia coli strain MSK.19.28, the Bacteroides xylanisolvens strain comprises Bacteroides xylanisolvens strain MSK.20.34, the Lacrimispora celerecrescens strain comprises Lacrimispora celerecrescens strain MSK.15.50, the Bacteroides caccae strain comprises Bacteroides caccae strain MSK.18.53, the Blautia faecis strain comprises Blautia faecis strain MSK.17.74, the Blautia obeum strain comprises Blautia obeum strain MSK.13.37, the Clostridium scindens strain comprises Clostridium scindens strain MSK.5.24, the Bifidobacterium strain comprises Bipzdobacterium sp. MSK.13.1, the Megasphaera massiliensis strain comprises Megasphaera massiliensis strain MSK.20.21, the Coprococcus comes strain comprises Coprococcus comes strain MSK.17.59, the Mitsuokella jalaludinii strain comprises Mitsuokella jalaludinii strain MSK.21.47, and/or the Blautia producta strain comprises Blautia producta strain MSK.4.17.

3. The composition of claim 1, wherein the bacterial population is an isolated bacterial population.

4. The composition of claim 1, wherein the bacterial population comprises at least two different bacterial strains.

5. The composition of claim 1, wherein the bacterial population comprises at least 16 different bacterial strains.

6-12. (canceled)

13. The composition of claim 1, further comprising a pharmaceutically acceptable excipient.

14-20. (canceled)

21. A method for treating or prophylactically treating an infection in a patient, the method comprising administering a therapeutically effective amount of a composition to the patient, thereby treating or prophylactically treating infection, wherein the composition comprises a bacterial population comprising at least one bacterial strain, wherein the bacterial strain is a Clostridium innocuum strain, a Clostridium symbiosum strain, a Collinsella aerofaciens strain, an Escherichia coli strain, a Bacteroides xylanisolvens strain, a Lacrimispora celerecrescens strain, a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, a Blautia producta strain, or any combination thereof.

22. (canceled)

23. The method of claim 21, wherein the infection is caused by an Enterobacteriaceae sp.

24. The method of claim 21, wherein the infection is caused by a drug resistant Enterobacteriaceae sp. or a multi-drug resistant Enterobacteriaceae sp.

25. The method of claim 21, further comprising administering one or more antibiotics to the patient.

26. A method for modulating one or more short chain fatty acids and/or one or more amino acids in a patient, the method comprising administering an effective amount of a composition to the patient, wherein the composition comprises a bacterial population comprising at least one bacterial strain, wherein the bacterial strain is a Clostridium innocuum strain, a Clostridium symbiosum strain, a Collinsella aerofaciens strain, an Escherichia coli strain, a Bacteroides xylanisolvens strain, a Lacrimispora celerecrescens strain, a Bacteroides caccae strain, a Blautia faecis strain, a Blautia obeum strain, a Clostridium scindens strain, a Bifidobacterium strain, a Megasphaera massiliensis strain, a Coprococcus comes strain, a Mitsuokella jalaludinii strain, a Blautia producta strain, or any combination thereof.

27. (canceled)

28. The method of claim 26, wherein the composition comprises at least two different bacteria strains.

29. The method of claim 26, wherein the composition comprises at least 46 different bacteria strains.

30. The method claim 26, wherein the composition comprises the bacterial strains of Block 1.

31. The method of claim 26, wherein the composition comprises the bacterial strains of Block 2.

32. The method of claim 26, wherein the composition comprises the bacterial strains of Block 3.

33. The method of claim 26, wherein the composition comprises the bacterial strains of Block 4.

34. The method of claim 26, wherein the composition comprises the bacterial strains of Block 5.

35. The method of claim 26, wherein the composition comprises the bacterial strains of Block 1 and Block 5.


SEQUENCE LISTING (16 sequences)

SEQ ID NO: 1 - DNA, 20 bp, synthetic construct
  source 1..20: mol_type = other DNA
agagtttgatcctggctcag

SEQ ID NO: 2 - DNA, 20 bp, synthetic construct
  source 1..20: mol_type = other DNA
agagtttgatcmtggctcag

SEQ ID NO: 3 - DNA, 23 bp, synthetic construct
  source 1..23: mol_type = other DNA
actgctgcsycccgtaggagtct

SEQ ID NO: 4 - DNA, 21 bp, synthetic construct
  source 1..21: mol_type = other DNA
gactcctacgggaggcwgcag

SEQ ID NO: 5 - DNA, 19 bp, synthetic construct
  source 1..19: mol_type = other DNA
gtattaccgcggctgctgg

SEQ ID NO: 6 - DNA, 19 bp, synthetic construct
  source 1..19: mol_type = other DNA
gtgccagcmgccgcggtaa

SEQ ID NO: 7 - DNA, 18 bp, synthetic construct
  source 1..18: mol_type = other DNA
ggattagataccctggta

SEQ ID NO: 8 - DNA, 20 bp, synthetic construct
  source 1..20: mol_type = other DNA
ggactacvsgggtatctaat

SEQ ID NO: 9 - DNA, 20 bp, synthetic construct
  source 1..20: mol_type = other DNA
ccgtcaattcctttragttt

SEQ ID NO: 10 - DNA, 23 bp, synthetic construct
  source 1..23: mol_type = other DNA
taaaactyaaakgaattgacggg

SEQ ID NO: 11 - DNA, 15 bp, synthetic construct
  source 1..15: mol_type = other DNA
yaacgagcgcaaccc

SEQ ID NO: 12 - DNA, 15 bp, synthetic construct
  source 1..15: mol_type = other DNA
gggttgcgctcgttg

SEQ ID NO: 13 - DNA, 19 bp, synthetic construct
  source 1..19: mol_type = other DNA
ggttaccttgttacgactt

SEQ ID NO: 14 - DNA, 20 bp, synthetic construct
  source 1..20: mol_type = other DNA
cggttaccttgttacgactt

SEQ ID NO: 15 - DNA, 35 bp, synthetic construct
  source 1..35: mol_type = other DNA
nnnnnnnnnnnnnnnnnnnnaytgggydtaaagng

SEQ ID NO: 16 - DNA, 38 bp, synthetic construct
  source 1..38: mol_type = other DNA
nnnnnnnnnnnnnnnnnnnnccgtcaattyhtttragt